(19) United States
(12) Patent Application Publication
(10) Pub. No.: US 2005/0170443 A1
(43) Pub. Date: Aug. 4, 2005

(54) Methods and use of binding components for improving assay specificity

(75) Inventor: Thomas L. Cantor, El Cajon, CA (US)

Correspondence Address: MORRISON & FOERSTER LLP, 3811 VALLEY CENTRE DRIVE, SUITE 500, SAN DIEGO, CA, 92130-2332, US

(73) Assignee: Scantibodies Laboratory, Inc., Santee, CA (US)

(21) Appl. No.: 10/958,541

(22) Filed: Oct. 4, 2004

Related U.S. Application Data
(60) Provisional application No. 60/508,547, filed on Oct. 3, 2003.
(60) Provisional application No. 60/573,475, filed on May 21, 2004.

(51) Int. Cl. G01N 33/53; G01N 33/537; G01N 33/543

(52) U.S. Cl. 435/7.92

(57) ABSTRACT

The present invention relates to detecting an analyte of interest in the presence of other analytes that may be of interest or moieties that interfere with normal assay protocols.

### PRIORITY CLAIM

This application claims priority benefit of U.S. Provisional Patent Application Nos. 60/508,547, filed Oct. 3, 2003 and 60/573,475, filed May 21, 2004 under 35 U.S.C. § 119(e), the contents of which are incorporated by reference in their entirety.

### TECHNICAL FIELD

The present invention relates to detecting an analyte of interest without detecting a similar analyte or moiety.

### BACKGROUND

Analytes such as parathyroid hormone (PTH) have historically presented problems in their accurate determination due to the frequent presence of interfering moieties and the low endogenous concentrations of many analytes. These moieties generally comprise molecules with considerable homology to the analyte of interest and, therefore, interfere by becoming bound and/or detected by assay reagents. Sometimes it is of interest to measure 2 analytes by use of one specific assay for one of the two analytes and another non specific assay that measures the sum (or total) of the 2 analytes. In these cases the concentration of the analyte measured with the specific assay is subtracted from the non specific assay value of the sum of the 2 analytes. The analyte not measured in the specific assay is the difference between the specific assay value and the non specific assay value. In the case of PTH, this subtraction method is used to derive the concentration of 7-84 PTH from the difference between the 1-84 PTH (measured in a specific assay) and total PTH (measured in a non specific assay). This type of subtraction method is also used to derive the concentration of LDL from the difference between HDL (measured in a specific assay) and total cholesterol (measured in a non specific assay) concentrations. An essential requirement to obtain an accurate value of the analyte derived from the difference between the specific assay value and the non specific or total assay value is that both of the analytes must be measured by the non specific (or total) assay. Equal concentrations of the 2 analytes must produce equal measurement values in the non specific (or total) assay. For example, 100 pgm/ml of 7-84 PTH must yield an assay value in the non specific total assay that is an equivalent value as would be yielded from 100 pgm/ml of 1-84 PTH in the same non specific total assay. This equimolar detection is sometimes challenging and this challenge constitutes the need to have direct measurements by specific assays rather than using the subtraction method.

1-84 PTH is generally understood as an important analyte for assessing calcium metabolism and bone turnover status in a subject. Similarly, 7-84 PTH has strong biological activities that are antagonistic to 1-84 PTH and is, therefore, an important hormone to measure, especially in end stage renal disease patients (to assess bone status). See, e.g., Faugere, M-C., et al., Kidney Int. (2001) 60:1460-8; Waller, S. C., et al., L. Am. Soc. Nephrol. (2003) 14:694; Divieti, P., et al., Endocrinology (2002) 143(1):171-6; Sneddon, W. B., et al., J. Biol. Chem. (2003) 278(44):43787-96; Langub, M., et al., Endocrinology (2003) 144(4):1135-38. While methods exist to directly measure 1-84 PTH, only indirect methods are currently utilized to assess 7-84 PTH levels, as 7-84 PTH is a part of 1-84 PTH, therefore, methods to measure 7-84 PTH or a fragment of PTH will also often measure 1-84 PTH.

The current method for measuring 7-84 PTH or another fragment of PTH involves two measurements. First, 1-84 PTH is measured in a sample with a highly specific 1-84 PTH assay, for example, the cyclase activating PTH (CAP, also referred to as whole PTH) assay manufactured by Scantibodies Laboratory, Inc. (Santee, Calif.). The next step is to assay the sample for 1-84 PTH plus 7-84 PTH or another fragment of PTH (or measuring for total PTH), with a total PTH assay (referred to as an “intact” PTH assay, but these “intact” PTH assays, in the past, have actually comprised a measurement of both 1-84 PTH plus 7-84 PTH and other fragments of PTH, if present). The total PTH assay often non-specifically measures 1-84 PTH and may also measure one or more PTH fragments of various sizes. 7-84 PTH, however, has been most frequently used to demonstrate the non specificity of these so-called “intact” PTH assays for the PTH fragment found in ESRD patients. Because intact PTH values are greater than specific 1-84 PTH assay values in ESRD patients and because HPLC has revealed a peak in ESRD patients corresponding by migration to 7-84 PTH, it is presumed that ESRD patients have large concentrations of 7-84 PTH. Thereafter the level of 7-84 PTH is obtained when the 1-84 PTH value is subtracted from the total PTH level. Because the 1-84 PTH assay value is measuring only 1-84 PTH, this “7-84 PTH” assay value could, in theory, be made up of any PTH fragment level from PTH₂₋₈₄ to PTH₂₈₋₈₄, although the presence of PTH fragments (other than 7-84 PTH) within this range has not been conclusively demonstrated.

The 1-84 PTH/7-84 PTH (and/or another fragment of PTH) ratio has been demonstrated to be a more accurate surrogate marker for bone biopsy analysis compared to other markers. In leading to the present disclosure it was discovered that the subtraction method amplifies variations in the 1-84 PTH/7-84 PTH ratio which are caused by minor percentage differences in large total PTH assay values compared to smaller 1-84 PTH assay values for the same samples resulting in large variations of the calculated 7-84 PTH )and/or another fragment) of PTH value. For at least this reason it is herein recognized as preferable to have a direct measurement of 7-84 PTH. This desired preference is analogous to the similar need to measure HDL directly instead of subtracting the measured LDL from total cholesterol in order to calculate the HDL/LDL ratio for the clinical assessment of the risk of atherosclerosis. This need was evidenced by the rapid adoption of a direct measurement of HDL by the clinical community when the direct measurement method became available.

Direct assay measurement of 7-84 PTH is problematic because the same epitopes that are present on the 7-84 PTH molecule are also present on the 1-84 PTH molecule, necessitating a co-measurement of 1-84 PTH whenever 7-84 PTH is measured. The shared epitopes of 1-84 PTH and 7-84 PTH (together with other PTH fragments) have inhibited the direct measurement of 7-84 PTH without the measurement of 1-84 PTH.

In the 1970's it was known that when serum calcium increases that the parathyroid gland secretes PTH fragments, although the function of these fragments was not understood. Recently the 7-84 PTH fragment has been documented to be inversely biologically active to PTH with respect to bone turnover, resorption, calcemia and osteoclast proliferation. Faugere et al. have demonstrated that the ratio of 1-84 PTH/Likely 7-84 PTH ratio decreases with increases in serum calcium. It has also been demonstrated that it is likely 7-84 PTH that is formed from within the parathyroid gland. Until the current observations, it has been assumed that there is only one form of 1-84 PTH. Furthermore, until recently the available understanding of 1-84 PTH and the different PTH fragments has been severely limited by the availability of only the “intact” PTH assay which measures both 1-84 and 7-84 PTH (and other fragments of PTH).

The immunoheterogeneous nature of circulating PTH has given rise over the past 40 years to ongoing replacements of PTH assays with increasing specificities. Under normal calcemic conditions, it is composed of 20% hPTH(1-84), the biologically-active form of the hormone on the PTH/PTHrP receptor, and of 80% carboxyl-terminal (C) fragments, considered until recently to be biologically inactive (D'Amour P, et al., Am. J. Physiol. 1986;251 (Endo Metab):E680-E687; D'Amour P, et al., J. Clin. Endocrinol. Metab. 1992;75:525-532; Brossard J H, et al., J. Clin. Endocrinol. Metab. 1996;81:3923-3929). In renal failure (RF), C-PTH fragments accumulate as they are in normal individuals mainly cleared by the kidney (D'Amour P, et al., Endocrinology 1985;117:127-134). It has been indicated that these C-PTH fragments represent more than 95% of circulating PTH (see Brossard J H, et al., supra). Studies in humans have also demonstrated the existence of smaller C-PTH fragments (D'Amour P, et al., J. Immunoassay 1989;10:191-205) and, more recently, of larger C-PTH fragments with a partially-preserved amino-terminal structure called non-(1-84)PTH. See Brossard J H, et al., J Clin Endocrinol Metab supra; Brossard J H, et al., J Clin Endocrinol Metab 1993;77:413-419; Lepage R, et al., Clin Chem 1998;44:805-809; Brossard J H, et al., Clin Chem 2000;46:697-703. The latter fragments were uncovered from HPLC analysis of circulating PTH with what was presumed at the time to be “intact” (I)-PTH assays (D'Amour P, et al., Endocrinology, supra; Brossard J H, et al., J Clin Endocrinol Metab 1993;77:413-419; Lepage R, et al., Clin Chem 1998;44:805-809; Brossard J H, et al., Clin Chem 2000;46:697-703). Non-(1-84)PTH accounts for 10% of C-PTH fragments and for 20% of I-PTH immunoreactivity in normal individuals (NI) (D'Amour P, et al., J Immunoassay, supra). In RF patients, it also accounts for greater than 10% of the C-PTH fragments, but for greater than 45% of I-PTH immunoreactivity (D'Amour P, et al., Endocrinology, supra; Lepage R, et al., Clin Chem 1998;44:805-809; Brossard J H, et al., Clin Chem 2000;46:697-703).

The importance of C-PTH fragments in PTH related biology has been disclosed in recent studies cited below. Human PTH(7-84), a surrogate for non-(1-84)PTH, and, to a lesser extent, smaller C-PTH fragments, have hypocalcemic, hypophosphatemic and hypophosphaturic effects as demonstrated in a thyroparathyroidectomized rat model and hPTH(7-84) antagonizes the hypercalcemic influence of hPTH(1-84) and hPTH(1-34) in the same model (Slatopolsky E, et al., Kidney Int 2000;58:753-761; Nguyen-Yamamoto L, et al., Endocrinology 2001;142:1386-1392). Furthermore, in vitro, hPTH(7-84) is a potent inhibitor of bone resorption induced by hPTH(1-84) and hPTH(1-34) and other PTH “agonists.” See Divieti P, et al., Endocrinology 2002;143:171-176. Both hPTH(7-84) and hPTH(39-84) have demonstrated activity consistent with their role as inhibitors of vitamin D-induced osteoclastogenesis. See id. These biological effects of C-PTH fragments are independent of the type 1 PTH/PTHrP receptor and studies indicate that their activity is exerted through a C-PTH receptor. See id.; Nguyen-Yamamoto L, et al., Endocrinology 2001 (supra).

Accordingly, there exists a need in the art for a method to measure 7-84 PTH and/or another PTH fragment without measuring 1-84 PTH. There also exists a need in the art for identifying and/or measuring a new molecular form of PTH (nfPTH). Moreover, the present inventor recognizes that other analytes of interest exist in the art, the measurement of which is confounded by any of a variety of interfering moieties which maintain homology to the analyte of interest and often cross-react with current reagents, and herein provides useful means to provide accurate exclusive measurement of these analytes without also measuring those interfering moieties. The present invention addresses these and other related needs in the art.

### DISCLOSURE OF THE INVENTION

In a frequent embodiment, the present disclosure provides a method for detecting an analyte in the presence of an interfering moiety comprising a) contacting a sample containing or suspected of containing an analyte and/or an interfering moiety with a blocking binding component to allow specific binding of the blocking binding component to the interfering moiety but not to the analyte, if the analyte and/or the interfering moiety is present in the sample; b) contacting the sample with a tracer binding component to allow specific binding of the tracer binding component to the analyte but not to the interfering moiety due to the presence of the blocking binding component bound thereon; and c) detecting the binding between the analyte and the tracer binding component to assess the presence and/or amount of the analyte in the sample, wherein the analyte is a fragment, analog or isoform of the interfering moiety and step a) is conducted prior to step b). Frequently, the steps of contacting the sample with a labeled tracer binding component and contacting the sample with an assay solid phase binding component are performed after the step comprising contacting the sample with the blocking binding component. Also frequently, the method further incorporates an assay solid phase binding component, wherein the assay solid phase binding component is contacted with the sample and allowed to specifically bind the analyte before detecting the binding between the analyte and the tracer binding component.

In a frequent embodiment, the interfering moiety contains an epitope that is not present, in whole or in part, on the analyte by virtue of the analyte's status as a fragment of the interfering moiety, wherein the blocking binding component is specific for this epitope. Often, the interfering moiety contains another epitope that overlaps the first epitope, wherein this other epitope is present on the analyte, and wherein the tracer binding component is specific for this other epitope.

Frequently, detection of the analyte of interest comprises determining the level of 7-84 PTH in the sample. On occasion, detection of the analyte of interest comprises determining the level of a PTH fragment other than, or in addition to, 7-84 PTH in the sample. Also frequently, the method further comprises determining a total PTH level in the sample, and wherein the 7-84 PTH level is subtracted from the total PTH level to determine the level of 1-84 PTH in the sample. A selection of two of the 7-84 PTH level, the total PTH level and the 1-84 PTH level are often compared in a ratio. The levels or the ratios are often used to diagnose, monitor or guide treatment for a disease or disorder.

In another frequent embodiment, a method for detecting an analyte in the presence of an interfering moiety is provided, comprising: a) contacting a sample containing or suspected of containing an analyte and/or an interfering moiety with an isolation binding component to allow specific binding of the isolation binding component to the interfering moiety but not to the analyte, if the analyte and/or the interfering moiety is present in the sample, wherein the interfering moiety is removed from a solution phase in the sample by binding with the isolation binding component; b) contacting the sample with a tracer binding component to allow binding of the tracer binding component to the analyte; and d) detecting the binding between the analyte and the tracer binding component to assess the presence and/or amount of the analyte in the sample, wherein the analyte is a fragment, analog or isoform of the interfering moiety and step a) is conducted prior to step b). Frequently, the method further comprises contacting the sample with an assay solid phase binding component to allow specific binding of assay binding component to the analyte but not to the interfering moiety.

Frequently, the interfering moiety is removed from solution via the formation of an interfering moiety complex that is formed upon the contact of the sample with a complex forming binding component capable of binding with the isolation binding component. Also frequently, the method further comprises contacting the sample with a nonspecific immunoglobulin composition that is derived from the same species as the isolation binding component, wherein the complex forming binding component is further capable of binding the nonspecific immunoglobulin composition to further form the interfering moiety complex. The isolation binding component comprises a mouse derived monoclonal antibody composition, the nonspecific immunoglobulin composition comprises mouse immunoglobulin, and the second immunoglobulin composition comprises goat anti-mouse immunoglobulin. On occasion, the nonspecific immunoglobulin is not derived from the same species as the isolation binding component, and the complex forming binding component may be a composition that is optionally has the capability of also binding the nonspecific immunoglobulin in addition to a capability of binding the isolation binding component.

In another frequent embodiment, the isolation binding component comprises a particle (comprising, for example, agarose, cellulose, glass fiber, magnetic particles, plastic surfaces, a microtiter plate, a glass slide, a nitrocellulose membrane, a cellulose derivative, a latex bead, a cell, an organelle, a protein or peptide, a test tube, a plastic bead, a colloidal gold particle, a colored particle, a magnetic bead, a quantum dot, a dipstick or a screen, etc.) attached to a binding component. Often the particle comprises an activated bead. The particle is often utilized when a nonspecific immunoglobulin is not employed.

Frequently, detection of the analyte comprises determining the level of 7-84 PTH in the sample. On occasion, detection of the analyte comprises determining the level of a PTH fragment in addition to, or other than, 7-84 PTH in the sample. Moreover, often the selective detection of the combination of the analyte and the interfering moiety in the sample comprises detecting the total PTH level in the sample. In this embodiment, the 7-84 PTH level is often subtracted from the total PTH level to determine the level of 1-84 PTH in the sample. In a frequent embodiment, a selection of two of the 7-84 PTH level, the total PTH level and/or the 1-84 PTH level are compared in a ratio. Also frequently, the analyte comprises calcitonin and the interfering moiety comprises procalcitonin or preprocalcitonin. The level(s) and/or the ratio(s) are often used to diagnose, monitor or guide treatment for a disease or disorder.

Frequently, the tracer binding component binds the interfering moiety, and the isolation binding component is removed from the reaction chamber after the binding component attached thereto binds the interfering moiety. However, removal is not required for the present methods. Also frequently, the method further comprises detecting the level of interfering moiety after removal from the reaction chamber and detecting the level of analyte in the reaction chamber after removal of the interfering moiety therefrom. The analyte often comprises 7-84 PTH, and/or another PTH fragment, and the interfering moiety comprises 1-84 PTH. Also often, the method further comprises calculating a total PTH level from the combined levels of 7-84 PTH and 1-84 PTH.

In a further frequent embodiment, a method for detecting an analyte in the presence of an interfering moiety is provided, comprising: a) placing a sample containing or suspected of containing an analyte and an interfering moiety in a reaction chamber; b) contacting the sample with the isolation binding component, wherein the isolation binding component specifically binds the interfering moiety but not an analyte in the sample; c) contacting the sample with a tracer binding component that binds the analyte and the interfering moiety in the sample; d) contacting the sample with an assay solid phase binding component that binds with the analyte in the sample; and e) selectively detecting the binding between the tracer binding component and: (i) the analyte, (ii) the interfering moiety, and/or (iii) the combination of the analyte and the interfering moiety, wherein the analyte is a fragment of the interfering moiety, wherein step b) is conducted prior to steps c) and/or d), and wherein the assay solid phase binding component bound to the analyte and/or the isolation binding component bound to the interfering moiety are optionally removed from the reaction chamber and into another chamber prior to selective detection of (i) the analyte or (ii) the interfering moiety.

Frequently, the assay solid phase binding component is removed from the reaction chamber after binding to the analyte and into another chamber prior to selective detection of the analyte or the interfering moiety, and the binding between the tracer binding component and the analyte and the binding between the tracer binding component and the interfering moiety are selectively determined, wherein such determination comprises determining the level of the analyte and the level of the interfering moiety in the sample.

Frequently, the analyte and the interfering moiety are present in the reaction chamber upon selective detection of the binding between the tracer binding component and (iii) the combination of the analyte and the interfering moiety. Also frequently, the analyte is removed from the reaction chamber after the sample is contacted with the isolation binding component. The analyte is often contacted with the tracer binding component and/or the assay solid phase binding component either (i) on contact with the other chamber, or (ii) subsequent to removal to the other chamber. The other chamber often comprises a vessel such as a test tube, wherein no notable binding reaction takes place.

Frequently, the detection of the analyte comprises determining the level of 7-84 PTH in the sample and detecting the interfering moiety comprises determining the level of 1-84 PTH in the sample. Often the binding between the tracer binding component and the analyte is detected in the other chamber. Frequently, the interfering moiety comprises another, or a second analyte. The method often further comprises calculating a total PTH level from the combined levels of 7-84 PTH, 1-84 PTH, and optionally another PTH fragment such as 1-34 PTH. Also frequently, a selection of two of the 7-84 PTH level, the total PTH level, the 1-84 PTH level and/or another PTH fragment level are compared in a ratio. The level(s) and/or the ratio(s) are often used to diagnose, monitor or guide treatment for a disease or disorder. Often these diseases include adynamic bone disease and high bone turnover as distinct from normal bone turnover.

In an occasional embodiment, the analyte comprises 7-84 PTH and the interfering moiety comprises 1-84 PTH, 1-34 PTH and/or 1-37 PTH, wherein the method further comprises determining the level of 1-84 PTH in the sample by a direct 1-84 PTH assay such as those known in the art (see below) or presented herein.

Frequently, the isolation binding component is attached to a wall of the reaction chamber such that upon placing at least a portion of the sample in the reaction chamber, the sample contacts the isolation binding component.

Also frequently, the tracer binding component further comprises a detectable label. On occasion, the tracer binding component is labeled with a detectable label and the tracer binding component comprises a first labeled tracer binding component that specifically binds the analyte and a second (or another) labeled tracer binding component that specifically binds the interfering moiety, wherein the label aspect of the first labeled tracer binding component is detectably distinguishable from the label aspect of the second labeled tracer binding component. Often, the first labeled tracer binding component is detectably distinguishable from the label aspect of the second labeled tracer binding component by virtue of the fact that the labeled tracer binding component/analyte of interest complex are removed from the first reaction chamber.

In a frequent embodiment, the disease or disorder is selected from osteoporosis, kidney stone disease or renal osteodystrophy. Also frequently, the present methods are used for prognosis, diagnosis and/or treatment monitoring of familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, Graves disease, the extent of parathyroid gland surgical removal, oversuppression with vitamin D or a vitamin D analogue, calcium or a calcimimetic and chronic uremia. Often, the hyperparathyroidism is primary hyperparathyroidism caused by primary hyperplasia or adenoma of one or more of the parathyroid glands or secondary hyperparathyroidism caused by renal failure. Also frequently, binding between the analyte and/or interfering moiety and the binding component is assessed by a format selected from the group consisting of, e.g., an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, immuno-radio metric assay (IRMA), μ-capture assay, linear flow membrane chromatography, inhibition assay, energy transfer assay, avidity assay, turbidometric immunoassay and time resolved amplified cryptate emission (TRACE) assay.

In another often included embodiment, the binding component aspect of each of the blocking binding component, the isolation binding component, the tracer binding component, and/or the assay solid phase binding component comprises an antibody, an antibody fragment or a member of a specific binding pair.

In a still further embodiment, an improvement is provided in an immunoassay of the type used to determine a total PTH level in a subject, the improvement comprising a means for a specific and direct determination of a 7-84 PTH level in a sample. On occasion, an improvement is provided in an immunoassay of the type used to determine a total PTH level in a subject, the improvement comprising a means for a specific and direct determination of a PTH fragment level in addition to, or other than, the 7-84 PTH level in a sample. Frequently, the total PTH level in the subject is comprised of a 1-84 PTH level, a 7-84 PTH level, and optionally PTH fragments other than 7-84 PTH.

Frequently, the binding component aspect of the blocking binding component, the tracer binding component, and the solid phase binding component comprises an antibody, an antibody fragment, a receptor, or a member of a specific binding pair.

In a frequent embodiment, the interfering moiety comprises a whole PTH and the analyte comprises a PTH fragment; wherein the interfering moiety comprises procalcitonin and the analyte comprises a preprocalcitonin fragment; wherein the interfering moiety comprises procalcitonin and the analyte comprises calcitonin; wherein the interfering moiety comprises gastric inhibitory polypeptide (GIP) and the analyte comprises glucagon-like peptide (GLP); wherein the interfering moiety comprises GIP-1 and the analyte comprises GLP-1; wherein the interfering moiety comprises GIP-2 and the analyte comprises GLP-2; wherein the interfering moiety is selected from an isoform of creatine kinase (CK) selected from the muscle (CK-MM), hybrid (CK-MB) and brain isoforms (CK-BB) and the analyte is selected from a CK isoform other than the interfering moiety; or wherein the interfering moiety comprises proinsulin and the analyte comprises insulin; wherein the interfering moiety comprises osteocalcin and the analyte comprises an osteocalcin fragment; or wherein the interfering moiety comprises adrenocorticotrophic hormone (ACTH) and the analyte comprises an ACTH fragment. In a frequent embodiment, the analyte comprises 7-84 PTH and the interfering moiety comprises 1-84 PTH.

In a frequent embodiment, the a label is present on the tracer binding component that is selected from the group consisting of a substrate, a chromogen, a catalyst, a chemiluminescent compound, a particulate label, a fluorescent label, an enzymatic label, a colorimetric label, a dye label, a radioactive label, and a magnetic label. Often, however, the tracer binding component is unlabeled, but is capable of being labeled to permit detection of the binding between the tracer binding component and a target ligand or other member of a specific binding pair. For example, the tracer binding component can comprise a specific mouse anti-human antibody that can be labeled via the introduction of a nonspecific label comprising an anti-mouse immunoglobulin. Other two step labeling processes are contemplated and known in the art.

Also frequently, the assay solid phase is selected from the group comprising a microtiter plate, a glass slide, a nitrocellulose membrane, a latex bead, a cell, a test tube, a plastic bead, a colloidal gold particle, a colored particle, a magnetic bead and a quantum dot, among others discussed herein.

In a further frequent embodiment, the present disclosure provides a novel method for specifically measuring 1-84 PTH. In this embodiment, frequently a method is provided for detecting whole PTH in a sample in the presence of PTH fragments comprising: a) contacting a sample containing or suspected of containing whole PTH and/or PTH fragments with a blocking binding component composition containing a binding component that specifically binds the whole PTH and the PTH fragment(s) in the sample, such that a unique epitope present on the whole PTH but not on the PTH fragments is left unbound by the binding component in the blocking binding component composition; b) contacting the sample with a tracer binding component to allow binding of the tracer binding component with the unique epitope on the whole PTH left unbound by the blocking binding component composition, wherein the tracer binding component does not bind the PTH fragment(s); and c) detecting the binding between the whole PTH and the tracer binding component to assess the presence and/or amount of the whole PTH in the sample, wherein step a) is conducted prior to step b). Frequently, the blocking binding component composition comprises a series of monoclonal antibodies or a polyclonal antibody, and wherein the monoclonal or polyclonal antibodies are directed against all or part of a region comprising 9-34 PTH. Also frequently, the tracer binding component comprises an antibody directed against all or part of a region comprising 1-34 PTH. In an often included embodiment, the method further comprises contacting an assay solid phase binding component with the sample and allowed to specifically bind the whole PTH and/or the PTH fragment(s) before detecting the binding between the whole and the tracer binding component.

In another embodiment, a composition is provided that is useful for the pretreatment of a sample to determine the level of 7-84 PTH in the sample using a total PTH assay as described herein, comprising a binding component specific for all or a part of a region on the PTH molecule comprising 1-9 PTH or 1-15 PTH, wherein the binding component is attached to a solid phase. In a further embodiment, another composition is provided that is useful for the pretreatment of a sample to determine the level of 1-84 PTH in the sample using a total PTH assay as contemplated herein, comprising a binding component specific for all or a part of a region on the PTH molecule comprising 15-34 PTH or 7-34 PTH, wherein the binding component is attached to a solid phase. The above composition may further comprise a means for assaying the total PTH.

In yet another embodiment, a method is provided for detecting whole PTH in a sample comprising: a) contacting a fluid sample containing or suspected of containing whole PTH and/or PTH fragments with a tracer binding component to allow specific binding of the tracer binding component to the whole PTH; b) contacting the sample with an isolation binding component to allow specific binding of the isolation binding component to the whole PTH; c) contacting the sample with a nonspecific binding component, wherein the nonspecific binding component is derived from the same species as the isolation binding component; d) contacting the sample with a complex forming binding component to allow binding of the complex forming binding component to the isolation binding component bound to the whole PTH and the nonspecific binding component to form a complex, wherein the complex precipitates out of solution; and e) detecting the binding between the tracer binding component and the whole PTH, wherein any or all of steps a), b) or c) are conducted prior to step d).

In a further embodiment, a method is provided for detecting PTH and fragments or analogs thereof in a sample comprising: a) contacting a sample containing or suspected of containing whole PTH and an N-terminal PTH fragment or analog with an isolation binding component to allow specific binding of the isolation binding component to the whole PTH but not to the N-terminal PTH fragment or analog; b) contacting the sample with an assay solid phase binding component to allow specific binding of the assay solid phase binding component to the N-terminal PTH fragment or analog but not to the whole PTH; c) contacting the sample with a tracer binding component to allow binding of the tracer binding component to the whole PTH and the N-terminal PTH fragment or analog; and d) detecting the binding between the tracer binding component and the whole PTH, the N-terminal PTH fragment or analog and/or the combination of the whole PTH and the N-terminal PTH fragment or analog, wherein step a) is conducted prior to steps b) and/or c).

In yet a further embodiment a method is provided for detecting a non-typical PTH (also referred to as non-typical 1-84 PTH or new form of PTH (nfPTH)) in a sample, comprising: a) assaying a sample using a total PTH assay that permits detection of 1-84 PTH and 7-84 PTH, if present in the sample, to determine a total PTH level in the sample; b) assaying the sample using a whole PTH assay that specifically detects 1-84 PTH, and also measures ntPTH, if present in the sample, to determine a combined level of 1-84 PTH and ntPTH in the sample; c) assaying the sample according to a method of one of the above embodiments to determine a 7-84 PTH level in the sample; and d) subtracting the difference between the 7-84 level and the total PTH level from the combined level of 1-84 PTH and ntPTH to determine the ntPTH level in the sample. Using an appropriate reagent specificity selection, steps a), b), and/or c) can be performed by assorted methods described herein. Thus, this embodiment frequently comprises a combination of one or more other embodiments described elsewhere herein.

The level of 7-84 PTH can be determined by any suitable methods. For example, the level of 7-84 PTH can be determined by a method comprising: a) contacting a sample containing or suspected of containing an analyte and/or an interfering moiety with a blocking binding component to allow specific binding of the blocking binding component to the interfering moiety but not to the analyte, if the analyte and/or the interfering moiety is present in the sample; b) contacting the sample with a tracer binding component to allow specific binding of the tracer binding component to the analyte but not to the interfering moiety due to the presence of the blocking binding component bound thereon; and c) detecting the binding between the analyte and the tracer binding component to assess the presence and/or amount of the analyte in the sample, wherein the analyte is a fragment, analog or isoform of the interfering moiety and step a) is conducted prior to or concurrently with step b).

In another example, the level of 7-84 PTH can be determined by a method for detecting an analyte in the presence of an interfering moiety comprising: a) contacting a sample containing or suspected of containing an analyte and/or an interfering moiety with an isolation binding component to allow specific binding of the isolation binding component to the interfering moiety but not to the analyte, if the analyte and/or the interfering moiety is present in the sample, wherein the interfering moiety is removed from a solution phase in the sample by binding with the isolation binding component; b) contacting the sample with a tracer binding component to allow binding of the tracer binding component to the analyte; and c) detecting the binding between the analyte and the tracer binding component to assess the presence and/or amount of the analyte in the sample, wherein the analyte is a fragment, analog or isoform of the interfering moiety and step a) is conducted prior to or concurrently with step b).

In still another example, the level of 7-84 PTH can be determined by a method comprising: a) placing a sample containing or suspected of containing an analyte and an interfering moiety in a reaction chamber; b) contacting the sample with an isolation binding component, wherein the isolation binding component specifically binds the interfering moiety but not the analyte in the sample; c) contacting the sample with a tracer binding component that binds the analyte and the interfering moiety in the sample; d) contacting the sample with an assay solid phase binding component that binds with the analyte in the sample; and e) selectively detecting the binding between the tracer binding component and: (i) the analyte, (ii) the interfering moiety, and/or (iii) the combination of the analyte and the interfering moiety, wherein the analyte is a fragment, analog or isoform of the interfering moiety, wherein step b) is conducted prior to steps c) and/or d), and wherein the assay solid phase binding component bound to the analyte and/or the isolation binding component bound to the interfering moiety are optionally removed from the reaction chamber and into another chamber prior to selective detection of (i) the analyte or (ii) the interfering moiety.

Frequently, a selection of two of the 7-84 PTH level, the 1-84 PTH level, the total PTH level and/or the ntPTH level are compared in a ratio. Also frequently, the ratio is used to diagnose, monitor or guide treatment for a disease or disorder such as adynamic bone disease, high bone turnover disease or osteoporosis.

Frequently, the measured total PTH level, 1-84 PTH level, 7-84 PTH level, 1-34 PTH level, ntPTH level, the level of another PTH fragment, or a ratio calculated therefrom are entered into an algorithm to evaluate the risk that the subject will develop a renal disease or disorder. The algorithm is generated utilizing patient data and accounts for multiple variables. For example, the algorithm may include an assessment of one or more of: 7-84 PTH levels, 1-84 PTH levels, total PTH levels, 1-34 PTH levels, ntPTH levels, levels of another PTH fragment, or ratios of selected PTH components (e.g., total PTH, 7-84 PTH, 1-84 PTH, 1-34 PTH, ntPTH, or another PTH fragment). In general, the use of an algorithm provides an example mode of correlating one or more variables with the risk that a subject will develop or has a disease or disorder as contemplated herein. The algorithm methodology is useful for any of the analytes contemplated herein (e.g., PTH, calcitonin, etc.) to monitor, diagnose and/or guide treatment for a disease or disorder.

Also frequently, a method is provided for monitoring, diagnosing and/or guiding treatment for a disease or disorder comprising evaluating the level of a specific analyte or interfering moiety in a sample, wherein if the measured analyte or interfering moiety level is at (or above or below) a specific, often pre-designated, level the subject is at risk for or has a specific disease or disorder. Often the analyte (and the level thereof), the interfering moiety (and the level thereof), and/or the specific disease or disorder is/are pre-designated. Further, based on the measured level of the analyte or interfering moiety, it is often determined that a ratio of the one or more analytes and/or interfering moieties should be utilized to monitor, diagnose and/or guide treatment for a disease or disorder. In this embodiment, the level of the analyte and/or interfering moiety is utilized as a gate indicating when the use of a ratio-based evaluation of the sample would be appropriate or medically indicated. For example, often the level of an analyte or interfering moiety may be present at a specific level (often a high or low level) that it provides an indication of a specific disease or disorder, without resorting to a ratio-based analysis.

In one embodiment, a method is provided for detecting an analyte in the presence of an interfering moiety comprising: a) contacting a sample containing or suspected of containing an analyte and/or an interfering moiety with a blocking binding component to allow specific binding of the blocking binding component to the interfering moiety but not to the analyte, if the analyte and/or the interfering moiety is present in the sample; b) contacting the sample with an analyte analog and a binding component to allow competitive binding of the analyte and the analyte analog to the binding component wherein either the analyte analog or the binding component comprises a detectable label, and the binding component does not specifically bind to the interfering moiety due to the presence of the blocking binding component bound thereon; and c) detecting the competitive binding of the analyte and the analyte analog to the binding component to assess the presence and/or amount of the analyte in the sample, wherein the analyte is a fragment, analog or isoform of the interfering moiety and step a) is conducted prior to or concurrently with step b).

In another embodiment, a method is provided for detecting an analyte in the presence of an interfering moiety comprising: a) contacting a sample containing or suspected of containing an analyte and/or an interfering moiety with an isolation binding component to allow specific binding of the isolation binding component to the interfering moiety but not to the analyte, if the analyte and/or the interfering moiety is present in the sample, wherein the interfering moiety is removed from a solution phase in the sample by binding with the isolation binding component; b) contacting the sample with an analyte analog and a binding component to allow competitive binding of the analyte and the analyte analog to the binding component wherein either the analyte analog or the binding component comprises a detectable label; and c) detecting the competitive binding of the analyte and the analyte analog to the binding component to assess the presence and/or amount of the analyte in the sample, wherein the analyte is a fragment, analog or isoform of the interfering moiety and step a) is conducted prior to or concurrently with step b).

In efforts to purify and sequence non-(1-84)PTH, modified HPLC acetonitrile gradients have been utilized to better separate non-(1-84)PTH from hPTH(1-84). In doing so, a new amino-terminal PTH molecular form has been revealed, which is distinct from PTH(1-84) and non-(1-84)PTH, and is detected only by a cyclase-activating (CA)-PTH assay, that is specific for the first 4 amino acids of the PTH structure (Gao P, et al., J Bone Min Res 2001;16:605-614). Information provided herein indicates that this new form of PTH (nfPTH) behaves biologically in the same manner as both a full length PTH(1-84) having an intact N-terminal sequence for binding and activation of the PTH1R, and an intact C-terminal sequence for binding and activation of the C-PTH receptor.

To separate non-(1-84)PTH from PTH(1-84), new HPLC gradients have been developed and have observed that the peak of immunoreactivity co-eluting with hPTH(1-84) in prior gradients can now be further separated into 2 entities when measured with a cyclase-activating PTH (CAP or CA-PTH) assay with unique label antibody specificity for the amino acids at and near the N-terminus of the PTH structure. As provided herein, this new resolution was investigated via the analysis of sera from 6 normal individuals (NI), 5 patients with primary hyperparathyroidism (PHP), and 8 pools of sera from renal failure (RF) patients with increasing PTH concentrations. A total (T)-PTH assay (measuring both PTH and non-(1-84) PTH) with label antibody recognition in the (15-34) region, the CA-PTH assay, and a carboxyl-terminal (C)-PTH assay (measuring both PTH and non-(1-84) PTH and C terminal fragments of PTH) specific for the (65-84) region were used to measure basal PTH values and to analyze aliquots from HPLC elutions. As expected, the T-PTH results were higher than the CA-PTH results in all NI (3.13±0.37 vs 2.29±0.33 pmol/L, p<0.01). Since CA-PTH was similar or higher than CA-PTH in ⅗ patients with PHP, the difference between the 2 assays was minimal (25.7±26 vs 23.1±24.1 pmol/L, NS). The difference between T-PTH and CA-PTH was more marked and present in all RF patients (47±35 vs 33.3±26.1 pmol/L, p<0.01). The CA-PTH assay recognized a peak of immunoreactivity migrating in front of hPTH(1-84) that was different from the non-(1-84) peak recognized by the T-PTH assay. This peak represented 8.13±2.45% (range 4.7-12) of CA-PTH immunoreactivity in NI, 24.7±23.3% (range 9-63.3) in PHP patients and 22.2±7.3% (range 17.3-35) in RF patients. This peak was not evident in the T-PTH assay but was by the C-PTH assay, suggesting structural integrity of the N-terminal (e.g., PTH₁₋₄, PTH₁₋₆, PTH₁₋₉) and PTH₆₅₋₈₄ regions but with a modification in the region comprising amino acid positions (15-34) responsible for the non-reactivity in the T-PTH assay. A post-translational modification within this region is consistent with our findings. Although not intending to be bound by theory, it is our current understanding that such a modification could comprise phosphorylation of the serine residue at position 17 in the PTH structure. Further studies contemplated herein elucidate the structure of this new amino-terminal PTH molecule and the attendant biological implications.

In one embodiment a method is provided for identifying a new molecular form of PTH (nfPTH), which method comprises: (a) obtaining a biological sample from a subject; (b) measuring the PTH level in said sample through the practice of one or more PTH immunoassays, wherein each of said one or more PTH immunoassays utilizes an antibody that specifically binds a PTH peptide region comprising an N-terminal region, a mid-terminal region, a C-terminal region, or a combination thereof, wherein each of said one or more PTH immunoassays utilizes an antibody that specifically binds a different PTH peptide region, or combination thereof, from that of any other of said one or more PTH immunoassays; (c) comparing the PTH levels measured by said one or more PTH immunoassays; and (d) identifying the presence of a nfPTH in said sample based on said comparing of the PTH levels measured by said one or more PTH immunoassays. Frequently, the nfPTH identified by the present embodiment is isolated. The isolation of the nfPTH often occurs via High Performance Liquid Chromatography (HPLC) as described herein.

In another embodiment, a composition is provided comprising a nfPTH. Frequently such composition is identified by the methods presented herein.

In a frequent embodiment, a method is provided for determining and/or monitoring the biological activity of an isolated amino-terminal PTH molecule isolated by the present methods, comprising: conducting a bioassay to determine the effect the amino-terminal PTH molecule has on osteoblast, osteoclast and/or chondrocyte activity. See, e.g., Loveridge, N., et al., Endocrinology 128(4):1938-46 (1991). On occasion such determining and/or monitoring is achieved via evaluating PTH agonist or PTH antagonist activity of the amino-terminal PTH molecule, wherein the effect of the amino-terminal PTH molecule on alkaline phosphatase and/or glucose 6-phosphate dehydrogenase activity in osteoclast and osteoclast cells is monitored.

In another frequent embodiment, a method is provided for measuring the circulating levels of a nfPTH, comprising: (a) isolating a nfPTH molecule; (b) generating an antibody specific for the nfPTH molecule; (b) obtaining a biological sample from a subject; (c) contacting said sample with said antibody such that said antibody binds nfPTH molecule, if present, in the sample; and (d) measuring the antibody bound to the nfPTH molecule.

In a further embodiment, a method is provided to identify a condition or disease in a sample comprising (a) obtaining a biological sample; (b) measuring two or more PTH component levels selected from a total PTH level, PTH fragment level, whole PTH level, or a nfPTH level in said sample; (c) comparing the measured levels of the two or more PTH component levels to identify a condition or disease in the sample. Frequently, the comparison is in the form of a ratio or proportion. On occasion, the PTH component comprising nfPTH is measured and a condition or disease is identified based thereon. In a frequent embodiment, the condition or disease pertains to a bone turnover related condition or disease such as primary hyperparathyroidism, adynamic low bone turnover, normal bone turnover, high bone turnover, osteoporosis, hypercalcemia, hypocalcemia, osteopetrosis, among others. In an occasional embodiment, such methods are useful to monitor and guide treatment for such conditions or diseases, wherein the levels of one or more PTH components are utilized to determine and/or guide therapy in a subject afflicted with such conditions or diseases.

In another embodiment, a method for treatment of a bone turnover related disorder is provided, comprising administering a pharmaceutical composition to a subject in need thereof, wherein said pharmaceutical composition comprises an isolated nfPTH molecule, together with a physiologically acceptable excipient. Frequently, the bone turnover related disorder is adynamic low bone turnover or osteoporosis; but, on occasion, the bone turnover related disorder is hyperparathyroidism or renal osteodystrophy.

### BRIEF DESCRIPTION OF THE DRAWINGS

FIG. 1(A-D) provides a depiction of an exemplary method utilizing specificity by blocking (SBB) technology.

FIG. 2(A-D) provides a depiction of an exemplary method utilizing selective epitope exposure (SEE) technology.

FIG. 3(A-G) provides a depiction of another exemplary method utilizing SBB technology to measure 1-84 PTH.

FIG. 4(A-F) provides a depiction of an exemplary method utilizing Cyclase Activating Parathyroid Hormone—Precipitating Removal (CAP-PR) technology

FIG. 5(A-D) provides a depiction of an exemplary method utilizing Analyte Removal by Insolubulisation (ARI) technology.

FIG. 6(A-E) provides a depiction of an exemplary method utilizing the Trio Kit technology.

FIG. 7(A-H) provides a depiction of an exemplary method utilizing precipitating reagent technology to measure 1-84 PTH.

FIG. 8(A-E) provides a depiction of an exemplary method of detecting large N-terminal PTH fragments and analogs such as 1-34 PTH (or teriparatide) and 1-84 PTH.

FIG. 9(A-F) provides a depiction of an exemplary method utilizing PCT-PR technology using precipitating antibodies.

FIG. 10(A) provides an HPLC profile measuring 7-84 PTH, 1-84 PTH and ntPTH percentages and levels in a subject. FIG. 10(B-H) provides a depiction of an exemplary method of measuring 7-84 PTH, 1-84 PTH and ntPTH levels.

FIG. 11 provides characteristics of PTH assays used in this study with PTH standards, and a HPLC profile from a patient with primary hyperparathyroidism. Standards were hPTH(1-84) ●, hPTH(7-84) ◯, [tyr³⁴] hPTH(19-84) Δ, hPTH(39-84) ▴, hPTH(39-68) □, hPTH(53-84) ▪, and hPTH(64-84) ♦. The CA-PTH assay reacted only with hPTH(1-84) and recognized a peak of immunoreactivity co-eluting with hPTH(1-84) in position 45 and a new peak of immunoreactivity in position 42. The T-PTH assay reacted with hPTH(1-84) and hPTH(7-84) similarly and also recognized hPTH(1-84) in position 45 and non-(1-84)PTH in positions 36 to 41. The C-PTH assay reacted mainly with hPTH (39-84) and less with hPTH(1-84) and hPTH(7-84). The C-PTH recognized HPLC peaks identified by the 2 other assays as well as several less hydrophobic peaks (positions 15, 16, 19, 23, 30).

FIG. 12 provides HPLC profiles of circulating PTH in various populations detected by the T-PTH and CA-PTH assays. Acetonitrile gradient 2 of FIG. 3 is used. Individual results are outlined as the light lines, and mean results for a group as the darker, heavier lines. The results are qualitatively similar to FIG. 1, but quantitatively different for each group. The planimetric evaluation of these peaks is summarized in Table 2.

FIG. 13 illustrates influence of various acetonitrile gradients on the separation of circulating PTH molecular forms by HPLC, using the serum of a patient with primary hyperparathyroidism and 3 different PTH assays. The T-PTH assay detected hPTH(1-84) (position 45) and non-(1-84)PTH (positions 36 to 41). The CA-PTH assay also detected hPTH(1-84) (position 45) and a peak migrating in front of hPTH(1-84) (positions 42, 43) different from non-(1-84) PTH. This peak reacted in the C-PTH assay while non-(1-84)PTH was much less reactive.

FIG. 14 provides HPLC profiles of oxidized hPTH(1-84) (left) and hPTH(7-84) (right). Acetonitrile gradient 2 (shown in FIG. 3) was used. Results with the Ca-PTH (dashed line) and T-PTH (solid line) assays. The usual positions of hPTH(1-84) and hPTH(7-84) are indicated by arrows. Oxidized hPTH(1-84) migrated in position 38, whereas oxidized hPTH(7-84) in migrated in position 36. Oxidized hPTH(1-84) reacted equally in the CA-PTH and T-PTH assays, whereas oxidized hPTH(7-84) reacted only in the T-PTH assay.

### DETAILED DESCRIPTION OF THE INVENTION

For clarity of disclosure, and not by way of limitation, the detailed description of the invention is divided into the subsections that follow.

A. Definitions

Unless defined otherwise, all terms of art, notations and other scientific terms or terminology used herein have the same meaning as is commonly understood by one of ordinary skill in the art to which this invention belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “antigen” refers to any compound capable of binding to an antibody, or against which antibodies can be raised.

As used herein, “antibody” refers to a polypeptide substantially encoded by an immunoglobulin gene or immunoglobulin genes, or fragments thereof. The recognized immunoglobulin genes include the kappa, lambda, alpha, gamma, delta, epsilon, and mu constant regions, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, an antibody is an immunoglobulin having an area on its surface or in a cavity that specifically binds to and is thereby defined as complementary with a particular spatial and polar organization of another molecule. The antibody can be polyclonal or monoclonal. Antibodies may include a complete immunoglobulin or fragments thereof. Fragments thereof may include Fab, Fv and F(ab′)2, Fab′, and the like. Antibodies may also include chimeric antibodies or fragment thereof made by recombinant methods.

As used herein, “monoclonal antibody” refers to an antibody obtained from a population of substantially homogeneous antibodies, i.e., the antibodies comprising the population are identical except for possible naturally occurring mutations that are present in minor amounts.

As used herein, “polypeptide” refers to a polymer of at least about 4, 5, 6, 7, or 8 amino acids. Throughout the specification, standard three letter or single letter designations for amino acids are used. In the art, this term is often used interchangeably with “peptide” or “protein.”

As used herein, “whole parathyroid hormone” or “whole PTH” refers to the complete molecule of PTH or a variant, fragment, derivative or analog thereof. Often this molecule stimulates osteoclast formation, osteoblast formation, bone resorption, stimulation of adenylate cyclase and bone turnover to increase blood calcium levels. 1-84 PTH is an example of whole PTH. For purposes herein, the name “parathyroid hormone (PTH)” is used, although all other names are contemplated. Other names of PTH include, for example, parathormone and parathyrin. Whole PTH assay values may be obtained by measuring a sample with a variety of assays. Whole PTH refers to any of a variety of species dependent forms of the PTH molecule. See, e.g., Caetano, A. R., et al., Equus Genome Res. 9(12): 1239-1249 (1999) (horse), U.S. patent application Publication US 2002/0110871 A1 (rat, mouse, bovine, canine, porcine), U.S. Pat. Nos. 6,689,566 and 6,743,590 (human). It is intended to encompass whole PTH with conservative amino acid substitutions that do not substantially alter its biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., MOLECULAR BIOLOGY OF THE GENE, 4th Edition, 1987, The Bejamin/Cummings Pub. Co., p. 224).

As used herein, “parathyroid hormone agonist” or “PTH agonist” refers to the complete molecule of PTH or a variant, fragment, derivative or analog thereof. Often this molecule stimulates osteoclast formation, osteoblast formation, bone resorption, stimulation of adenylate cyclase and bone turnover to increase blood calcium levels. Whole PTH, e.g., 1-84 PTH, is an example of a PTH agonist, but other PTH agonists are contemplated. A PTH agonist further refers to peptides which have PTH agonist properties. It is intended to encompass a PTH agonist with conservative amino acid substitutions that do not substantially alter its biological activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al., MOLECULAR BIOLOGY OF THE GENE, cited supra).

As used herein, “parathyroid hormone antagonist” or “PTH antagonist” refers to a PTH fragment or derivative having biological actions that counter all or part of the effects of a PTH agonist, and/or has its own biological activity independent of a PTH agonist. 7-84 PTH is an example of a PTH antagonist. As further described below, a variety of other examples of PTH antagonists are contemplated. This term frequently includes a PTH fragment or derivative that lacks PTH agonist biological activity. It is intended to encompass a PTH antagonist with conservative amino acid substitutions that do not substantially alter its activity. Suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al. MOLECULAR BIOLOGY OF THE GENE, cited supra).

Conservative amino acid substitutions can frequently be made in a protein without altering either the conformation or the function of the protein. Such changes include substituting any of isoleucine (I), valine (V), and leucine (L) for any other of these hydrophobic amino acids; aspartic acid (D) for glutamic acid (E), and vice versa; glutamine (Q) for asparagine (N) and vice versa; and serine (S) for threonine (T), and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments. See, e.g., BIOCHEMISTRY 13-15 (L. Stryer ed., 2d ed. 1981); Henikoff et al., PNAS (1992) 89:10915-10919; Lei et al., J. Biol. Chem. (1995) 270 (20):11882-6.

As used herein, the terms “total PTH” refers to a combination of whole PTH and PTH fragments in a subject. Alternatively, “total PTH” refers to a combination of PTH agonist and PTH antagonist in a subject. Often this “combination” refers to a measurement of the levels of each of the substituents of the total PTH in a subject. This measurement frequently comprises detecting 1-84 PTH and 7-84 PTH in a sample. Often this measurement comprises detecting 1-84 PTH, 7-84 PTH and another PTH fragment, such as 1-34 PTH or 1-37 PTH, in a sample. Occasionally, total PTH measurement includes measuring ntPTH.

As used herein, the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues comprise an aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, the term “subject” is not limited to a specific species or sample type. For example, the term “subject” may refer to a patient, and frequently a human patient. However, this term is not limited to humans and thus encompasses a variety of mammalian species.

As used herein, the term “specifically binds” refers to the binding specificity of a specific binding pair. Recognition by an antibody of a particular target in the presence of other potential targets is one characteristic of such binding. “Binding component member” refers to a member of a specific binding pair, i.e., two different molecules wherein one of the molecules specifically binds with the second molecule through chemical or physical means. The two molecules are related in the sense that their binding with each other is such that they are capable of distinguishing their binding partner from other assay constituents having similar characteristics. The members of the binding component pair are referred to as ligand and receptor (antiligand), specific binding pair (sbp) member and sbp partner, and the like. A molecule may also be a sbp member for an aggregation of molecules; for example an antibody raised against an immune complex of a second antibody and its corresponding antigen may be considered to be an sbp member for the immune complex.

In addition to antigen and antibody binding component members, other binding components include, as examples without limitation, biotin and avidin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence or the entire protein, polymeric acids and bases, dyes and protein binders, peptides and specific protein binders (e.g., ribonuclease, S-peptide and ribonuclease S-protein), metals and their chelators, and the like. Furthermore, binding components can include members that are analogs of the original binding component member, for example, an analyte-analog or a binding component member made by recombinant techniques or molecular engineering.

If the binding component is an immunoreactant it can be, for example, an antibody, antigen, hapten, or complex thereof. If an antibody is used, it can be a monoclonal or polyclonal antibody, a recombinant protein or antibody, a chimeric antibody, a mixture(s) or fragment(s) thereof, as well as a mixture of an antibody and other binding component members. The details of the preparation of such antibodies and their suitability for use as specific binding members are known to those skilled in the art.

As used herein, “label” refers to any substance which is capable of producing a signal that is detectable by visual or instrumental means. Various labels suitable for use in the present invention include labels which produce signals through either chemical or physical means. Such labels can include enzymes and substrates, chromogens, catalysts, fluorescent compounds, chemiluminescent compounds, and radioactive labels. Other labels include particulate, fluorescent, enzymatic, colorimetric, dye, radioactive, magnetic or other such labels known in the art. Other suitable labels include particulate labels such as colloidal metallic particles such as gold, colloidal non-metallic particles such as selenium or tellurium, dyed or colored particles such as a dyed plastic or a stained microorganism, organic polymer latex particles and liposomes, colored beads, polymer microcapsules, sacs, erythrocytes, erythrocyte ghosts, or other vesicles containing directly visible substances, and the like. Typically, a visually detectable label is used as the label component of the label reagent, thereby providing for the direct visual or instrumental readout of the presence or amount of the analyte in the test sample without the need for additional signal producing components at the detection sites.

The selection of a particular label is not critical to the present invention, but the label will be capable of generating a detectable signal either by itself, or be instrumentally detectable, or be detectable in conjunction with one or more additional signal producing components, such as an enzyme/substrate signal producing system. A variety of different label reagents can be formed by varying either the label or the specific binding member component of the label reagent; it will be appreciated by one skilled in the art that the choice involves consideration of the analyte to be detected and the desired means of detection.

Exemplary labels further include biotin (detectable by binding to labeled avidin or streptavidin) and enzymes, such as horseradish peroxidase or alkaline phosphatase (detectable by addition of enzyme substrates to produce a colored reaction product).

Detection methods generally depend on the type of label. A radioisotope-labeled probe or target nucleic acid can be detected by autoradiography. Alternatively, the probe or the target nucleic acid labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As used herein, “detectible signal” is used in its broadest sense. This term refers to a signal produced in the described methods, compositions and kits that is detectable by observation using instrumentation or otherwise. The signal need not be a visual signal. Without limitation, the type of signal produced depends on the label reagents used.

As used herein, “particle” refers to a solid phase, or non-fluid phase, moiety. Particles of the present disclosure are distinguishable from fluid sample (e.g., insoluble). The term frequently refers to a gel, particularly a polymer gel such as an agarose gel. This term also refers to a bead; and any of a variety of bead materials are contemplated. On occasion, the term “particle” refers to a portion of a solid phase such as a wall of a tube. In general, a particle of the present invention is capable of being affixed with binding components and labels, often through the use of a linker. Frequently the bond between a binding component and a particle comprises a covalent bond, but non-covalent bonds are also suitable.

As used herein the term “avoids binding” refers to the specificity of particular binding components such as antibodies or antibody fragments. Antibodies or antibody fragments that avoid binding a particular moiety generally contain a specificity such that a large percentage of the particular moiety would not be bound by such antibodies or antibody fragments. This percentage generally lies within the acceptable cross reactivity percentage with interfering moieties of assays utilizing antibodies directed to detecting a specific target. Frequently, antibodies or antibody fragments of the present disclosure avoid binding greater than about 90% of an interfering moiety, although higher percentages are clearly contemplated and preferred. For example, antibodies or antibody fragments of the present disclosure avoid binding about 91%, about 92%, about 93%, about 94%, about 95%, about 96%, about 97%, about 98%, about 99%, and about 99% or more of an interfering moiety. Less occasionally, antibodies or antibody fragments of the present disclosure avoid binding greater than about 15%, or about 50%, or about 60%, or about 70%, or greater than about 75%, or greater than about 80%, or greater than about 85% of an interfering moiety. Relatedly, the above description is equally true of particular binding components that specifically bind an interfering moiety rather than an analyte of interest, although the specificity is reversed.

As used herein, “disease or disorder” refers to a pathological condition in an organism resulting from, e.g., infection or genetic defect, and characterized by identifiable symptoms.

As used herein, “high bone turnover” refers to the bone turnover rate as being above a normal bone turnover rate in a subject and is one of the symptoms manifested in subjects having hyperparathyroidism. While not bound by theory, a subject afflicted with severe hyperparathyroidism has a higher bone turnover rate than the same subject afflicted with mild hyperparathoidism, however, both having a high bone turnover rate as compared with a normal subject and a subject afflicted with adynamic bone disease.

As used herein, the term “N-terminal” refers to the amino terminus of a polypeptide, such as a PTH polypeptide, having a free amino group. With reference to a PTH fragment, an N-terminal PTH fragment refers to a non-whole contiguous portion of PTH having an intact N-terminal. An “intact N-terminal” as used herein refers to PTH or a PTH fragment having an intact 1st position of PTH₁₋₈₄. This first position is also referred to herein as an “original N-terminus” or an “original N-terminal.”

As used herein, the term “C-terminal” refers to the carboxyl terminus of a polypeptide, such as a PTH polypeptide, having a free carboxyl group. With reference to a PTH fragment, a C-terminal PTH fragment refers to a non-whole contiguous portion of PTH having an intact C-terminal. An “intact C-terminal” as used herein refers to PTH or a PTH fragment having an intact 84th position of PTH₁₋₈₄. This 84th position is also referred to herein as an “original C-terminus” or an “original C-terminal.”

As described herein, when the analyte is PTH, often the analyte comprises 7-84 PTH, but it is not limited to 7-84 PTH. Often the analyte comprises 1-84 PTH or whole PTH. Thus, as used herein one analyte is not automatically the analyte and the other the interfering moiety as the particular method dictates which is which. Moreover, although one analyte may be designated an interfering moiety and another analyte (or analyte of interest), the first of these may also be measured in accordance with the present methods. The distinction between analyte an interfering moiety is made for ease of presentation and is not limiting.

As used herein, “mammal” refers to any of the mammalian class of species. Frequently, the term “mammal,” as used herein, refers to humans, human subjects or human patients.

As used herein, a “functional derivative or fragment” of PTH agonist or PTH antagonist refers to a derivative or fragment of PTH that still substantially retains its function as a PTH agonist or PTH antagonist. Normally, the derivative or fragment retains at least 50% of its PTH agonist or PTH antagonist activity. Preferably, the derivative or fragment retains at least 60%, 70%, 80%, 90%, 95%, 99% and 100% of its PTH agonist or PTH antagonist activity. It is also possible that a functional derivative or fragment of PTH agonist or PTH antagonist has higher PTH agonist or PTH antagonist activity than a parent molecule from which the functional derivative or fragment is derived from.

B. Blocking Methodologies

1. Specificity By Blocking

In one embodiment, a method is provided for direct detection or measurement of an “analyte” or “analyte of interest” (both terms are used interchangeably herein) in the presence of an interfering moiety (e.g., an undesirable analyte to measure). In general, the interfering moiety and the analyte of interest each have distinguishing characteristics. For example, the analyte of interest may comprise 7-84 PTH, and the interfering moiety may comprise 1-84 PTH. On occasion, the analyte of interest may comprise a PTH fragment other than, or addition to, 7-84 PTH (such as 1-34 PTH or 1-37 PTH), and the interfering moiety comprises 1-84 PTH and/or a PTH fragment. These are polypeptides of different lengths that, owing to their homology, often cross-react or otherwise interfere with reagents intended to measure one or the other. Frequently, the analyte comprises a fragment, isoform or analog of the interfering moiety.

The binding component blocking methodology described herein provides for the measurement of a specific analyte of interest in a sample, even in the presence of an interfering moiety. Moreover, this method allows for the measurement of an analyte of interest that comprises a portion of a whole molecule that comprises the interfering moiety. For example, the analyte of interest can comprise 7-84 PTH and the interfering moiety can comprise 1-84 PTH. 7-84 PTH generally comprises a large polypeptide fragment of 1-84 PTH.

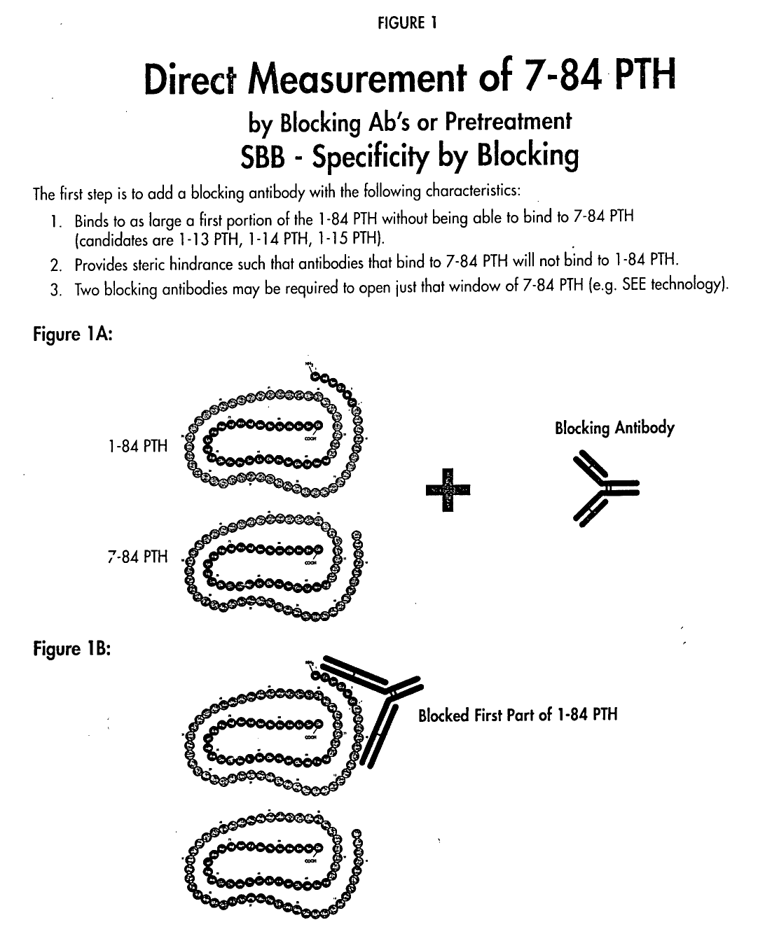

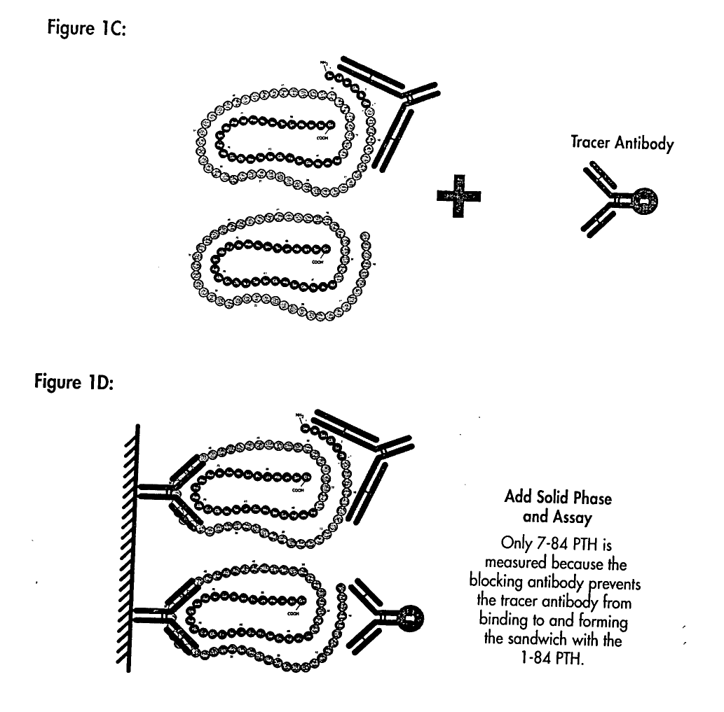

In one embodiment, a sample is obtained from a subject, which sample is suspected of containing an analyte of interest and an interfering moiety. Often one need not suspect that the sample contains these constituents, but such presence is merely assumed as they comprise typical constituents of such sample types. A blocking binding component composition is contacted with the sample under conditions that permit binding component binding. The binding component in the blocking binding component composition, which frequently comprises an antibody, is then allowed to bind the interfering moiety. The binding component has a specificity such that it generally will not bind the analyte of interest. A tracer binding component is then contacted with the sample, which tracer binding component binds an analyte of interest, but does not bind an interfering moiety that has been bound or blocked by the binding component. Steric hindrance or allosteric changes generally are responsible for the lack of binding between the interfering moiety that is bound by the blocking binding component and the tracer binding component. Frequently, the tracer binding component is labeled or capable of being labeled with a detectible label. The sample is then optionally contacted with an assay solid phase binding component that specifically binds the analyte of interest and/or the interfering moiety. Alternatively, the tracer may be added after the sample is contacted with the assay solid phase, or concurrently therewith. The assay solid phase binding component generally comprises a binding component attached to a solid phase. Thereafter the analyte of interest is detected through the detection of the binding between the analyte and the tracer binding component. Generally, if the tracer binding component is labeled, this detection is undertaken using means appropriate to detect the specific type of label on the tracer binding component. If the tracer binding component is not labeled, a labeling means is contacted with the sample and allowed to bind the tracer binding component. The labeling means may be a member of a specific binding pair comprising the tracer binding component and the labeling means, the tracer binding component can also be a specific or nonspecific ligand of the labeling means. Contemplated labeling means incorporate a detectible label in accordance with the description provided herein. An exemplary specificity by blocking process is depicted in FIG. 1. The analyte of interest is generally detected such that the presence, level and/or concentration is determined in the sample (and the subject).

In FIG. 1 an exemplary method is depicted wherein the first step is to add a blocking antibody to a sample having which (1) binds to as large a first portion of the 1-84 PTH without being able to bind to 7-84 PTH (candidates include 1-13 PTH, 1-14 PTH, 1-15 PTH, etc.); and (2) provides steric hindrance such that antibodies that bind to 7-84 PTH will not bind to 1-84 PTH. Also a solid phase is often contacted with the sample to conduct an assay to determine the concentration of the analyte, wherein, in this embodiment, only 7-84 PTH (the analyte) is measured because the blocking antibody prevents the tracer antibody from binding to and forming the assay sandwich of tracer binding component and solid phase assay binding component with the 1-84 PTH.

Multiple binding/blocking components are often utilized, each having a different specificity. Multiple binding components are useful, for example, when multiple interfering moieties or epitopes are present in the sample having distinguishing characteristics. Multiple binding components may also be useful when it is desired to utilize binding components having a specificity for a smaller portion of the interfering moiety and together they are utilized to bind a pre-designated portion of the interfering moiety or defined epitopes.

A blocking binding component is generally useful to block a portion of the interfering moiety to prevent or inhibit binding of that portion by a further assay binding component. On occasion, a blocking binding component is useful to prevent another binding component from binding the interfering moiety through allosteric effects resulting from the binding of the blocking binding component. Such allosteric effects may comprise a conformational change in the structure of the interfering moiety. Frequently, however, the blocking binding component inhibits or prevents binding by a further binding component due to steric hindrance.

In a frequent embodiment, the blocking binding component comprises an antibody. In a further embodiment, the interfering moiety comprises whole parathyroid hormone (PTH) or a fragment thereof and the blocking antibody comprises an antibody having a specificity for 1-3 PTH, 1-4 PTH, 1-5 PTH, 1-6 PTH, 1-7 PTH, 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 1-22 PTH, 1-23 PTH, 1-24 PTH, 1-25 PTH, 1-26 PTH, 1-27 PTH, 1-28 PTH, 1-29 PTH, 1-30 PTH, 1-31 PTH, 1-32 PTH, 1-33 PTH, 1-34 PTH, 2-5 PTH, 2-6 PTH, 2-7 PTH, 2-8 PTH, 2-9 PTH, 2-10 PTH, 2-11 PTH, 2-12 PTH, 2-13 PTH, 2-14 PTH, 2-15 PTH, 2-16 PTH, 2-17 PTH, 2-18 PTH, 2-19 PTH, 2-20 PTH, 2-21 PTH, 2-22 PTH, 2-23 PTH, 2-24 PTH, 2-25 PTH, 2-26 PTH, 2-27 PTH, 2-28 PTH, 2-29 PTH, 2-30 PTH, 2-31 PTH, 2-32 PTH, 2-33 PTH, 2-34 PTH, 3-6 PTH, 3-7 PTH, 3-8 PTH, 3-9 PTH, 3-10 PTH, 3-11 PTH, 3-12 PTH, 3-13 PTH, 3-14 PTH, 3-15 PTH, 3-16 PTH, 3-17 PTH, 3-18 PTH, 3-19 PTH, 3-20 PTH, 3-21 PTH, 3-22 PTH, 3-23 PTH, 3-24 PTH, 3-25 PTH, 3-26 PTH, 3-27 PTH, 3-28 PTH, 3-29 PTH, 3-30 PTH, 3-31 PTH, 3-32 PTH, 3-33 PTH, 3-34 PTH, 4-8 PTH, 4-9 PTH, 4-10 PTH, 4-11, PTH, 4-12 PTH, 4-13 PTH, 4-14 PTH, 4-15 PTH, 4-16 PTH, 4-17 PTH, 4-18 PTH, 4-19 PTH, 4-20 PTH, 4-21 PTH, 4-22 PTH, 4-23 PTH, 4-24 PTH, 4-25 PTH, 4-26 PTH, 4-27 PTH, 4-28 PTH, 4-29 PTH, 4-30 PTH, 4-31 PTH, 4-32 PTH, 4-33 PTH, 4-34 PTH, 5-9 PTH, 5-10 PTH, 5-11, PTH, 5-12 PTH, 5-13 PTH, 5-14 PTH, 5-15 PTH, 5-16 PTH, 5-17 PTH, 5-18 PTH, 5-19 PTH, 5-20 PTH, 5-21 PTH, 5-22 PTH, 5-23 PTH, 5-24 PTH, 5-25 PTH, 5-26 PTH, 5-27 PTH, 5-28 PTH, 5-29 PTH, 5-30 PTH, 5-31 PTH, 5-32 PTH, 5-33 PTH, 5-34 PTH, 6-9 PTH, 6-10 PTH, 6-11, PTH, 6-12 PTH, 6-13 PTH, 6-14 PTH, 6-15 PTH, 6-16 PTH, 6-17 PTH, 6-18 PTH, 6-19 PTH, 6-20 PTH, 6-21 PTH, 6-22 PTH, 6-23 PTH, 6-24 PTH, 6-25 PTH, 6-26 PTH, 6-27 PTH, 6-28 PTH, 6-29 PTH, 6-30 PTH, 6-31 PTH, 6-32 PTH, 6-33 PTH, 6-34 PTH, etc., among others, depending on the nature of the analyte of interest and the interfering moiety. Although specificity up to position 34 is described above, the blocking antibody will, on occasion, have a specificity for positions beyond 34 on the PTH molecule. In addition, combinations of blocking antibodies are contemplated comprising two or more antibodies having varying, but complementary specificities for the PTH molecule in accordance with the present methods.

A variety of analytes and interfering moieties are contemplated. For example, the interfering moiety frequently comprises a whole PTH and the analyte comprises a PTH fragment; or the interfering moiety comprises preprocalcitonin or fragment thereof and the analyte comprises procalcitonin; or the interfering moiety comprises procalcitonin and the analyte comprises calcitonin; or the interfering moiety comprises gastric inhibitory polypeptide (GIP) and the analyte comprises glucagon-like peptide (GLP); or the interfering moiety comprises GIP-1 and the analyte comprises GLP-1; or the interfering moiety comprises GIP-2 and the analyte comprises GLP-2; or the interfering moiety is selected from an isoform of creatine kinase (CK) selected from the muscle (CK-MM), hybrid (CK-MB) and brain isoforms (CK-BB) and the analyte is selected from a CK isoform other than the interfering moiety; or the interfering moiety comprises proinsulin and the analyte comprises insulin, or vice versa; wherein the interfering moiety comprises osteocalcin and the analyte comprises an osteocalcin fragment; or the interfering moiety comprises an adrenocorticotrophic hormone (ACTH) fragment and the analyte comprises ACTH.

As indicated above, the tracer binding component often comprises a binding component and a detectible label. A variety of label types are contemplated as set forth herein. In frequent embodiments, this tracer binding component is specific for the analyte of interest such that, upon contact with the sample under conditions that permit binding, the tracer binding component specifically binds the analyte of interest. The tracer binding component frequently is of a specificity that would bind an interfering moiety, but for the “bound” presence of the blocking binding component.

On occasion, the binding component of the tracer and the label component of the tracer comprise separate compositions such that after contacting the sample with the binding component of the tracer, and binding of this component to the analyte of interest, the detectible label component is contacted with the sample, which then binds or attaches to the binding component of the tracer. This is referred to as a two-step labeling process. The label may bind with the binding component of the tracer through any of a variety of means, for example, they may be (or may be attached to) corresponding members of a specific binding pair.

The tracer binding component can comprise a particle bound to a binding component. In such embodiments, the particle is generally detectible or capable of being detected when the labeled tracer binding component is bound to the analyte of interest. In one embodiment, the particle is a bead of the type that is capable of assay utilizing flow cytometry. Often, in this type of embodiment, the bead is a fluorescent bead or is itself attached to a label detectible using flow cytometry.

In a frequent embodiment, the tracer binding component comprises a labeled antibody. In a further embodiment, the analyte of interest comprises whole parathyroid hormone (1-84 PTH) or a fragment thereof and the tracer binding component comprises an antibody having a specificity for 1-3 PTH, 1-4 PTH, 1-5 PTH, 1-6 PTH, 1-7 PTH, 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 1-22 PTH, 1-23 PTH, 1-24 PTH, 1-25 PTH, 1-26 PTH, 1-27 PTH, 1-28 PTH, 1-29 PTH, 1-30 PTH, 7-15 PTH, 7-16 PTH, 7-17 PTH, 7-18 PTH, 7-18 PTH, 7-19 PTH, 7-20 PTH, 7-21 PTH, 7-22 PTH, 7-23 PTH, 7-24 PTH, 7-25 PTH, 7-26 PTH, 7-27 PTH, 7-28 PTH, 7-29 PTH, 7-30 PTH, 7-31 PTH, 7-32 PTH, 7-33 PTH, 7-34 PTH, 8-15 PTH, 8-16 PTH, 8-17 PTH, 8-17 PTH, 8-18 PTH, 8-19 PTH, 8-20 PTH, 8-21 PTH, 8-22 PTH, 8-23 PTH, 8-24 PTH, 8-25 PTH, 8-26 PTH, 8-27 PTH, 8-28 PTH, 8-29 PTH, 8-30 PTH, 8-31 PTH, 8-32 PTH, 8-33 PTH, 8-34 PTH, 9-15 PTH, 9-16 PTH, 9-17 PTH, 9-17 PTH, 9-18 PTH, 9-19 PTH, 9-20 PTH, 9-21 PTH, 9-22 PTH, 9-23 PTH, 9-24 PTH, 9-25 PTH, 9-26 PTH, 9-27 PTH, 9-28 PTH, 9-29 PTH, 9-30 PTH, 9-31 PTH, 9-32 PTH, 9-33 PTH, 9-34 PTH, etc., among others, depending on the nature of the analyte of interest. In the exemplary embodiment wherein the interfering moiety comprises 1-84 PTH and the analyte of interest comprises 7-84 PTH, the tracer binding component often avoids or is blocked from binding PTH fragments smaller than 7-84 PTH.

The assay solid phase binding component comprises a binding component attached or bound to a solid phase. The assay solid phase binding component frequently comprises an antibody attached to a solid phase. The assay solid phase can comprise a chamber wall, column wall, a tube wall, a plate, a well, a particle, a bead, a cell, an organelle, a protein or peptide, a dipstick, a screen, among other solid phases known in the art. The assay solid phase binding component is useful to bind and capture the labeled analyte of interest. Often the binding component aspect of the assay solid phase binding component specifically binds the analyte of interest but avoids binding the interfering moiety, however this is not necessary. Thus, often the binding component aspect of the assay solid phase binding component specifically binds the analyte of interest and the interfering moiety. Also often the binding component aspect of the assay solid phase binding component comprises an antibody specific for PTH, frequently falling within or comprising 39-84 PTH, a mid-terminal or C-terminal region of PTH. In a frequent embodiment, when multiple interfering moieties are present in a sample, the binding component aspect of the assay solid phase binding component specifically binds one or more of the interfering moieties, but does not bind others. In a frequent embodiment, the assay solid phase binding component is useful to isolate the labeled analyte of interest from the sample.

In one embodiment, the interfering moiety contains an epitope that is not present, in whole or in part, on the analyte by virtue of the analyte's status as a fragment of the interfering moiety, wherein the blocking binding component is specific for this epitope. Frequently, the interfering moiety contains another epitope that overlaps the first epitope, wherein this other epitope is present on the analyte, and wherein the tracer binding component is specific for this other epitope. For example, the first epitope may be 7-15 PTH and the interfering moiety epitope may comprise 1-9 PTH. As another example, the first epitope may comprise an epitope within positions 60-91 of the procalcitonin molecule (comprising three or more amino acid residues) and the interfering moiety epitope may comprise an epitope within positions 1-70 of the procalcitonin molecule (e.g., 1-65, 10-64, 20-63, 30-62, among others within those boundaries having the N-terminal of the epitope on the N-terminal side of position 60 of the procalcitonin molecule). Other overlapping epitopes may be present in the same or other analytes as described herein.

In another embodiment, the interfering moiety comprises a whole PTH and the analyte comprises a PTH fragment, or the analyte of interest comprises calcitonin and the analyte comprises a preprocalcitonin fragment; or the interfering moiety comprises procalcitonin and the analyte comprises calcitonin. Often, the detection of the analyte of interest comprises determining the level of the analyte of interest in the sample. Frequently, the analyte of interest comprises 7-84 PTH and the interfering moiety comprises 1-84 PTH. In an often included embodiment, the method further comprises determining a total PTH level in the sample, and wherein the 7-84 PTH level is subtracted from the total PTH level to determine the level of 1-84 PTH in the sample. In such an embodiment, a selection of two of the 7-84 PTH level, the total PTH level and the 1-84 PTH level are often compared in a ratio. This ratio is frequently used to diagnose, monitor or guide treatment for a disease or disorder. The gate, threshold and/or algorithm methods described herein may be utilized in the diagnosis, monitoring or guiding of treatment for a disease or disorder. The disease or disorder is often selected from the group consisting of osteoporosis, kidney stone disease, familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, Graves disease, the extent of parathyroid gland surgical removal, oversuppression with vitamin D or a vitamin D analogue or a calcimimetic or calcium and chronic uremia.

In another embodiment, the binding component aspect of the blocking binding component, the tracer binding component, and the assay solid phase binding component comprises an antibody, an antibody fragment, a receptor, or a member of a specific binding pair. Frequently, when the tracer binding component is labeled, the label on the labeled is selected from the group consisting of an enzyme and a substrate, a chromogen, a catalyst, a chemiluminescent compound, a particulate label, a fluorescent label, an enzymatic label, a colorimetric label, a dye label, a radioactive label, and a magnetic label. In another embodiment, the assay solid phase binding component often comprises an antibody having a specificity for the region 39-84 PTH.

2. Selective Epitope Exposure

In another embodiment, a method is provided for directly detecting an analyte of interest through selective epitope exposure in the presence of interfering moieties that usually or hold the potential to detrimentally affect assay specificity or accuracy. Selective epitope exposure is accomplished through the use of a combination of blocking binding components having different specificities. These blocking binding components bind the analyte of interest and/or the interfering moiety in a manner that exposes a specific epitope on the analyte of interest, but generally blocks one or more other epitopes present on the analyte of interest and/or the interfering moiety. The exposed epitope is then available for binding with another, frequently labeled, assay binding component.

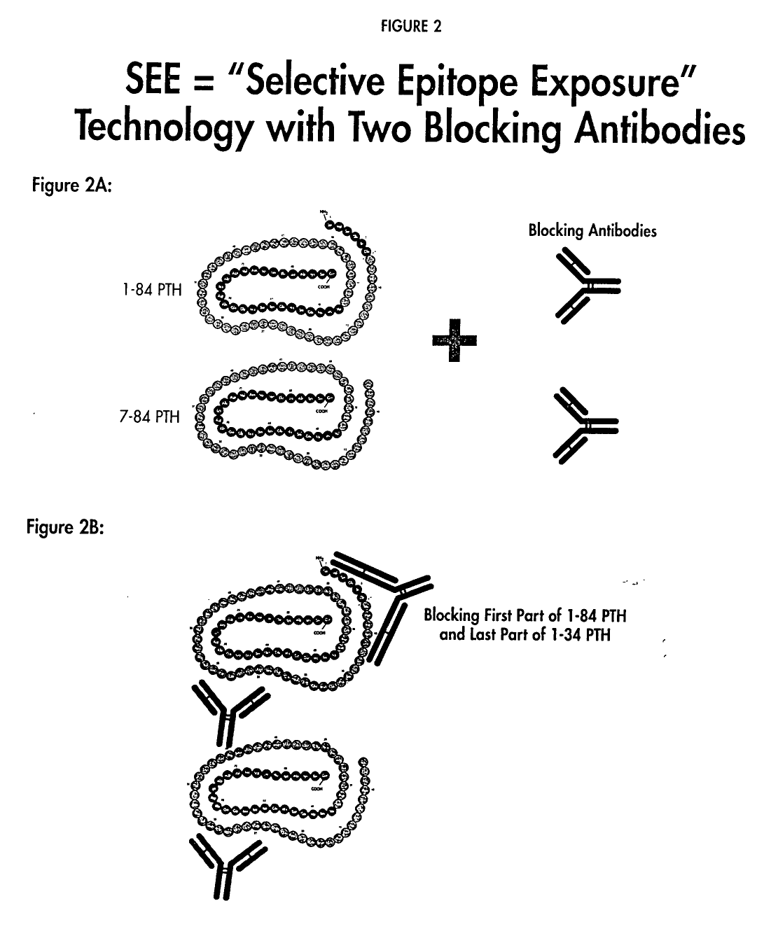

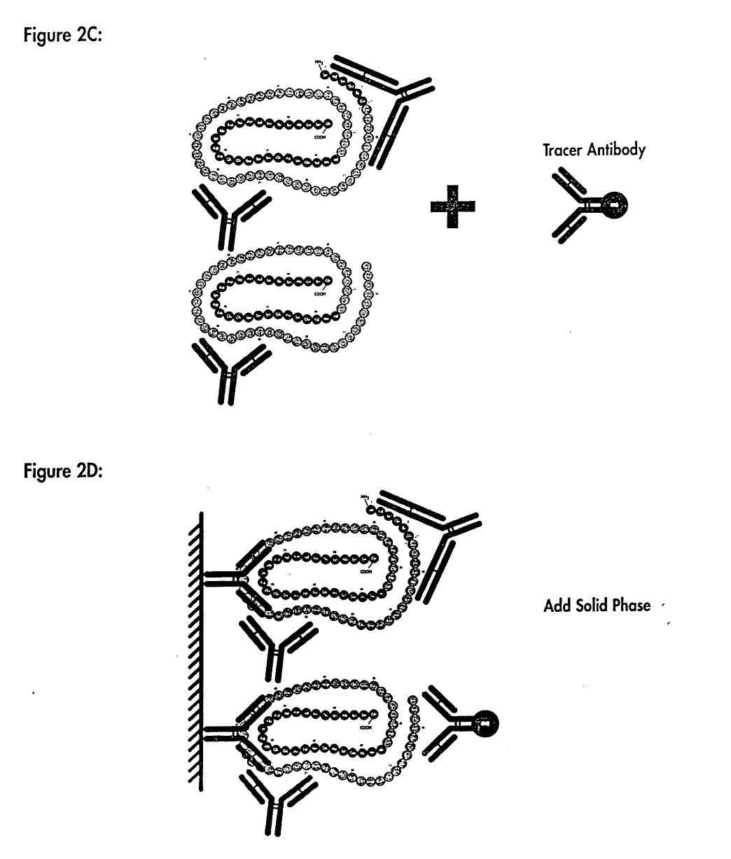

In a frequent embodiment, a sample is obtained from a subject, which sample may or may not contain an analyte and/or an interfering moiety. A blocking binding component composition is contacted with the sample. Thereafter the blocking binding components in the blocking composition are permitted to specifically bind the analyte and/or the interfering moiety. One or more regions of the analyte are left unbound by the blocking binding components. Thereafter the sample is contacted with a tracer binding component that specifically binds the epitope left unbound by the binding components in the blocking binding component composition. The tracer binding component generally does not bind the interfering moiety. In an often included embodiment, the sample is then contacted with an assay solid phase binding component that specifically binds the analyte and optionally the interfering moiety, which permits the assessment of the sample for the binding between the tracer binding component and the analyte. Thus, the analyte bound by the tracer binding component can be detected by one of several methods contemplated herein and known in the art. An exemplary selective epitope exposure process is depicted in FIG. 2.

The blocking binding components will often bind one or more epitopes on the analyte of interest, but will leave an unbound region comprising an epitope which is designed to be bound by the assay solid phase binding component. This unbound region is frequently unhindered by the binding of the blocking binding components either by steric hindrance or by allosteric changes. On occasion, the unbound region may be slightly altered but still available for binding with a specific assay binding component. In the example of PTH as the analyte of interest, the unbound region frequently comprises a region on the N-terminal side of the PTH molecule. In general, this region is left open for binding with a tracer binding component.

The blocking binding component composition frequently comprises multiple blocking binding components having different specificities. Also frequently, the blocking binding component composition comprises a single blocking binding component. On occasion, multiple blocking compositions are often utilized, each comprising a blocking binding component having a different or the same specificities. Frequently, in an embodiment wherein multiple blocking binding compositions are utilized these compositions may be contacted with the sample simultaneously or in sequence. On occasion, a first blocking binding composition is contacted with the sample and the blocking binding component(s) therein are allowed to bind the analyte and/or the interfering moiety in the sample, and after this binding another blocking binding composition is contacted with the sample and the blocking binding component(s) therein are allowed to bind the analyte and/or the interfering moiety in the sample, and so forth.

In one embodiment, the blocking binding component comprises an anti-PTH antibody specific for a particular region or epitope along the length of the PTH molecule. For example, one blocking binding component can comprise an antibody having a specificity for 1-3 PTH, 1-4 PTH, 1-5 PTH, 1-6 PTH, 1-7 PTH, 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 2-5 PTH, 2-6 PTH, 2-7 PTH, 2-8 PTH, 2-9 PTH, 2-10 PTH, 2-11 PTH, 2-12 PTH, 2-13 PTH, 2-14 PTH, 2-15 PTH, 2-16 PTH, 2-17 PTH, 2-18 PTH, 2-19 PTH, 2-20 PTH, 2-21 PTH, 3-6 PTH, 3-7 PTH, 3-8 PTH, 3-9 PTH, 3-10 PTH, 3-11 PTH, 3-12 PTH, 3-13 PTH, 3-14 PTH, 3-15 PTH, 3-16 PTH, 3-17 PTH, 3-18 PTH, 3-19 PTH, 3-20 PTH, 3-21 PTH, 4-8 PTH, 4-9 PTH, 4-10 PTH, 4-11, PTH, 4-12 PTH, 4-13 PTH, 4-14 PTH, 4-15 PTH, 4-16 PTH, 4-17 PTH, 4-18 PTH, 4-19 PTH, 4-20 PTH, 4-21 PTH, 5-9 PTH, 5-10 PTH, 5-11, PTH, 5-12 PTH, 5-13 PTH, 5-14 PTH, 5-15 PTH, 5-16 PTH, 5-17 PTH, 5-18 PTH, 5-19 PTH, 5-20 PTH, 5-21, 6-9 PTH, 6-10 PTH, 6-11, PTH, 6-12 PTH, 6-13 PTH, 6-14 PTH, 6-15 PTH, 6-16 PTH, 6-17 PTH, 6-18 PTH, 6-19 PTH, 6-20 PTH, 6-21 PTH, etc., among others. Further, another blocking binding component can be utilized comprising having a specificity for 25-34 PTH, among others such that a region from above about at least 4 amino acids in length is left unbound and relatively uninhibited by blocking binding component. Frequently this region comprises about 4 to about 10 or more amino acids in length. The blocking binding components are selected in order to bind to PTH, but to preserve an unbound region on the PTH polypeptide that can be bound by a separate tracer binding component. On occasion, the blocking binding component is directed against another or a different interfering moiety contemplated here, such as procalcitonin.

The tracer binding component frequently is of a specificity that permits specific binding with a particular, and often predetermined, region of an analyte of interest. Moreover, the tracer binding component frequently specifically binds with the epitope comprised in the region left unbound by the blocking binding component. This tracer binding component frequently comprises an antibody. Moreover, in the example of PTH as the analyte of interest, the tracer binding component frequently has a specificity for a region on the N-terminal side of the PTH molecule. This region frequently comprises one or more epitopes. This region on the N-terminal side of the PTH molecule is non-restrictive and frequently refers to a region on the N-terminal side of position 34 of the PTH molecule. Nevertheless, on occasion the tracer binding component is specific for a region on the C-terminal side of the PTH molecule. Similar to the tracer binding components described elsewhere herein, the tracer binding component comprises a detectible label and a binding component. In an occasional embodiment, the specificity of the tracer binding component and the assay solid phase binding component may be reversed. Although the specificity and constitution of the binding component may vary based on the particular assay type, the detectible label is intended to be generic and can be tailored to any of a variety of assay types. Thus, detectible labels discussed herein and known in the art are contemplated.

The assay solid phase binding component is similar to that described elsewhere herein. Features of the assay solid phase portion of this component are further described herein. Generally the specificity of the binding component aspect of the assay solid phase binding component will vary based on the desired analyte of interest. Moreover, this specificity may also vary based on the specificity chosen for the blocking binding components and/or the assay tracer binding components. Frequently, the assay solid phase binding component specifically binds a region other than that bound by the blocking and tracer binding components. In a frequent embodiment, another region (in addition to the region described above that is bound by a tracer binding component) on the analyte of interest is left unbound by the blocking binding component(s); the assay solid phase binding component will frequently specifically bind this “other” region. For example, the analyte of interest can be a PTH molecule such as 7-84 PTH and the binding component aspect of the assay solid phase binding component can be an antibody having specificity for 39-84 PTH. Also frequently the binding component aspect of the assay solid phase binding component comprises an antibody specific for PTH, frequently falling within or comprising 39-84 PTH, a mid-terminal or C-terminal region of PTH. In an occasional embodiment, the specificity of the assay solid phase binding component and the tracer binding component are reversed.

In another embodiment, the present disclosure contemplates any of a variety of analytes as analytes of interest. These are not restricted to PTH and not to particular fragments thereof. 7-84 PTH is frequently discussed herein as an exemplary analyte of interest, but the present disclosure clearly contemplates that the analyte of interest can be a fragment of PTH other than 7-84 PTH, or whole PTH (in addition to a variety of other analytes). In addition, the analyte can comprise calcitonin and the interfering moiety comprising procalcitonin. In such an example, an epitope falling within positions 60-91 of the procalcitonin molecule (comprising three or more amino acid residues) will be left open for binding with an assay tracer binding component. The blocking antibodies may be directed to portions on the N-terminal and C-terminal sides, and slightly overlapping but not encompassing, the epitope comprised in this region 60-91 to the extent that calcitonin when it is not incorporated into procalcitonin could be bound by the blocking antibodies.

As one of skill in the art would appreciate based on the present disclosure, the embodiments can be tailored to the specific analyte of interest.

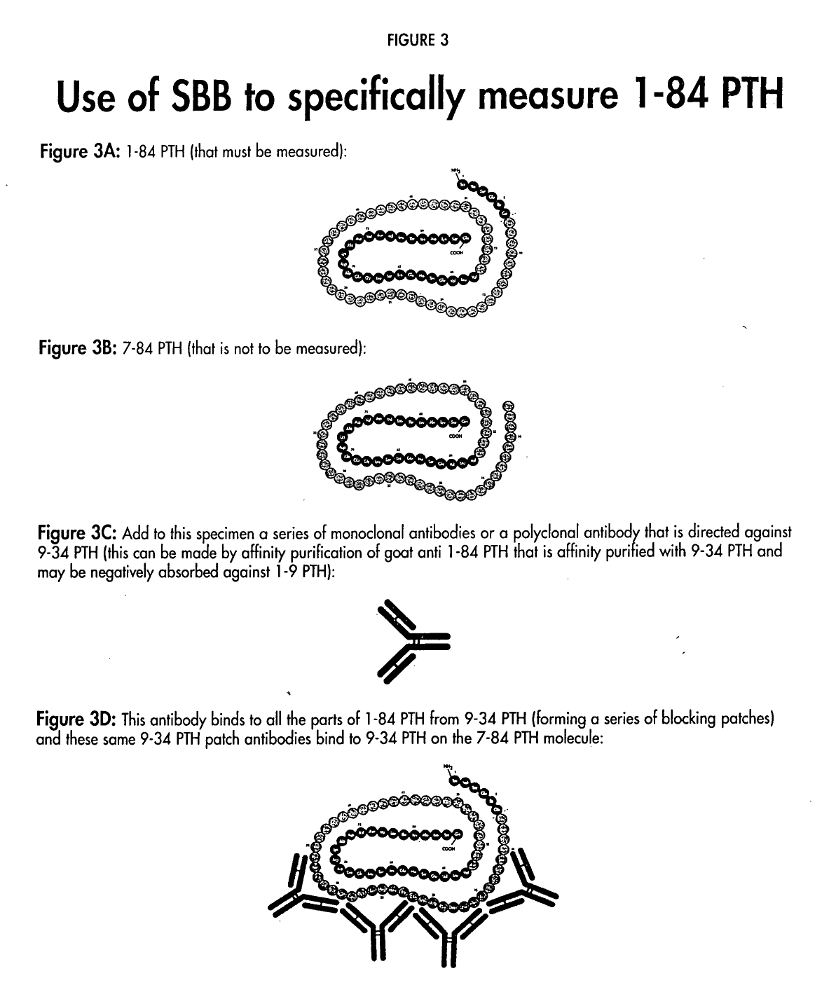

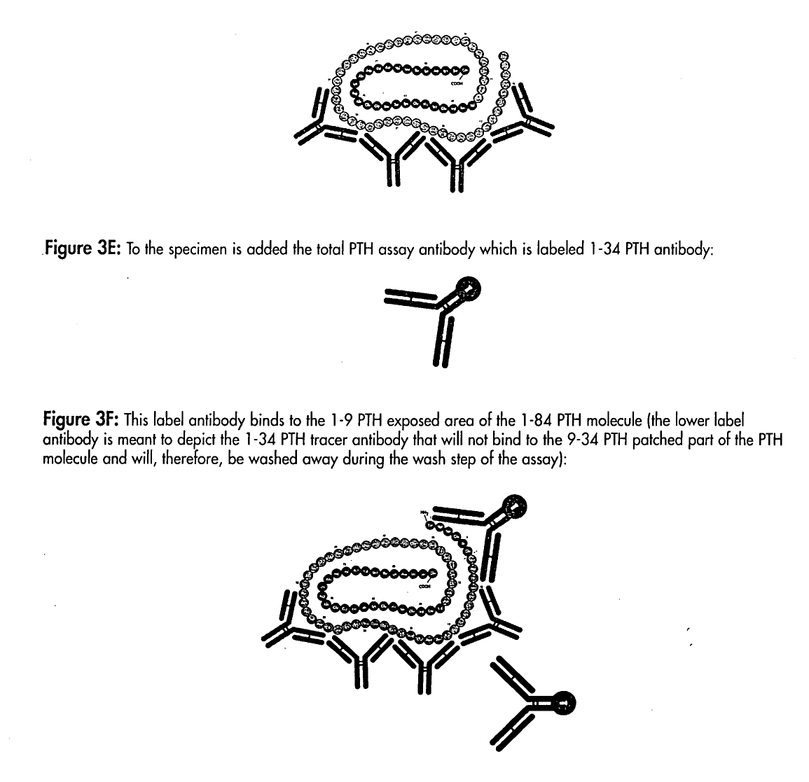

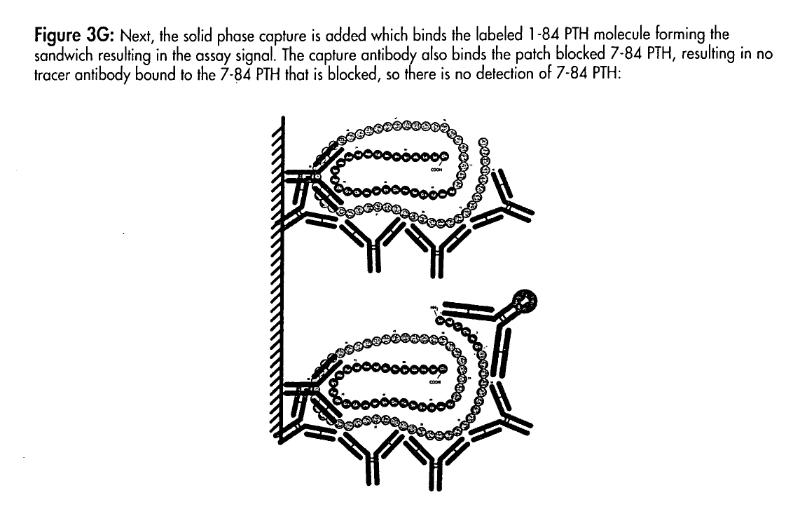

In one embodiment, the analyte of interest is 1-84 PTH and the interfering moiety comprises a large PTH fragment such as 7-84 PTH (as depicted in FIG. 3). In this embodiment one obtains a sample from a subject. A series of blocking monoclonal antibodies, a polyclonal antibody, a series of affinity purified polyclonal antibodies or one affinity purified polyclonal antibody directed against 9-34 are then contacted with this sample under conditions that would allow for antibody binding. The blocking antibody is then permitted to bind the analyte and/or the interfering moiety. This results in one or more antibodies binding 1-84 PTH and 7-84 PTH along all parts of the PTH molecule from position 9 to position 34. The sample is then contacted with a labeled tracer antibody specific for the N-terminus of the PTH molecule having a specificity such as 1-3 PTH, 1-4 PTH, 1-5 PTH, 1-6 PTH, 1-7 PTH, 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 1-22 PTH, 1-23 PTH, 1-24 PTH, 1-25 PTH, 1-26 PTH, 1-27 PTH, 1-28 PTH, 1-29 PTH, 1-30 PTH, 1-31 PTH, 1-32 PTH, 1-33 PTH or 1-34 PTH, among others. Generally, the tracer antibody is capable of selectively and specifically binding 1-84 PTH. The tracer antibody then binds the 1-84 PTH molecule at or around exposed positions 1-9. Notably, this tracer antibody will avoid binding 7-84 PTH as all binding regions or epitopes susceptible to binding with the tracer antibody would be occupied with the bound blocking antibody. In this embodiment, the assay solid phase capture antibody is frequently then contacted with the sample which binds the labeled 1-84 PTH and 7-84 PTH. The assay solid phase capture antibody can be specific for a region left exposed on the PTH molecule that is not bound by tracer or blocking antibody. For example, the assay solid phase capture antibody can be specific for a region within 39-84 PTH. Also often the binding component aspect of the assay solid phase binding component comprises an antibody specific for PTH, frequently falling within or comprising 39-84 PTH, a mid-terminal or C-terminal region of PTH. Thereafter the bound 1-84 PTH is detected through appropriate means depending, in part, on the type of label utilized.

An exemplary process utilizing SBB technology is depicted in FIG. 3(A-H). In this Figure a sample containing 1-84 PTH and 7-84 PTH is obtained. A series of monoclonal antibodies or a polyclonal antibody that is directed against 9-34 PTH (this can be made by affinity purification of goat anti 1-84 PTH or goat anti 1-34 PTH that is affinity purified with 9-34 PTH and may be negatively absorbed against 1-9 PTH) is then introduced to the sample (3C). The antibody binds to all the parts of 1-84 PTH from 9-34 PTH (forming a series of blocking patches) and these same 9-34 PTH patch antibodies bind to 9-34 PTH on the 7-84 PTH molecule (3D). In addition, a labeled 1-34 PTH antibody is added to the sample (3E). The labeled antibody binds to the 1-9 PTH exposed area of the 1-84 PTH molecule (the lower label antibody is meant to depict the 1-34 PTH tracer antibody that will not bind to the 9-34 PTH patched part of the PTH molecule and will, therefore, be washed away during the wash step of the assay) (3F). Next, the assay solid phase capture is added which binds the labeled 1-84 PTH molecule forming the sandwich of assay solid phase binding component—analyte—tracer binding component resulting in the assay signal (3G). The capture antibody also binds the patch blocked 7-84 PTH, resulting in no tracer antibody bound to the 7-84 PTH that is blocked, so there is no detection of 7-84 PTH.

In this embodiment, antibodies are utilized as the exemplary binding components but all other binding components contemplated herein may be utilized. Moreover, PTH is utilized as an exemplary embodiment, but other analytes of interest may be assayed by these methods. Aspects of the blocking binding component, tracer binding component and assay solid phase binding component and methods used for these components described elsewhere herein are applicable to the present methods to the extent that they fit within the general methodology set forth herein.

C. Isolation Binding

1. Analyte Removal by Insolubulisation (ARI)

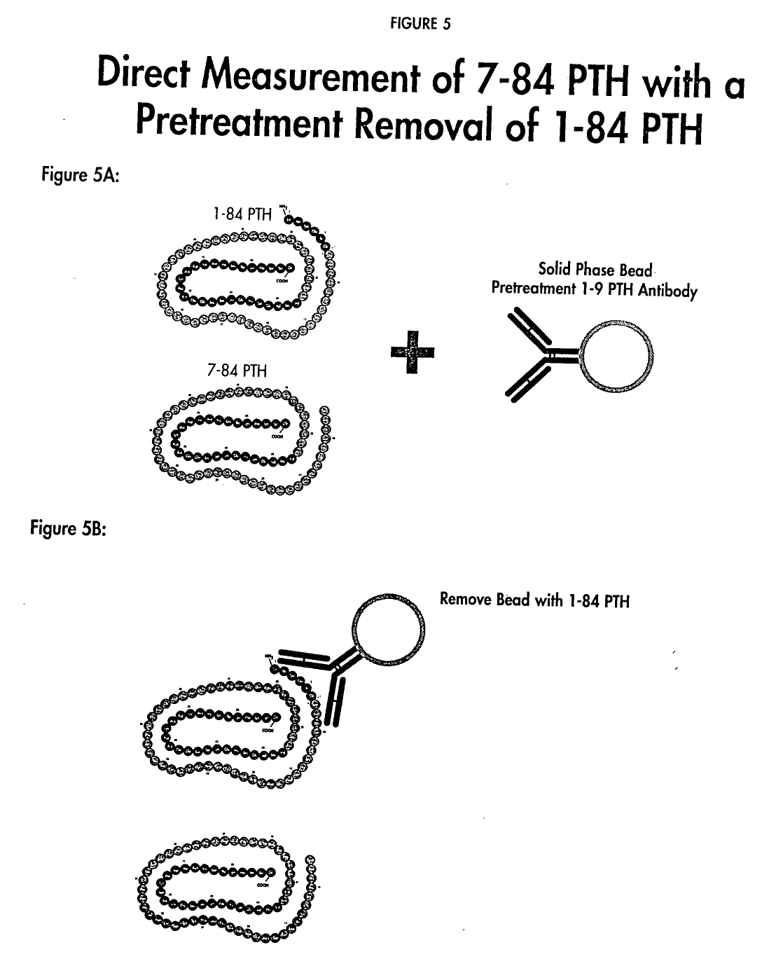

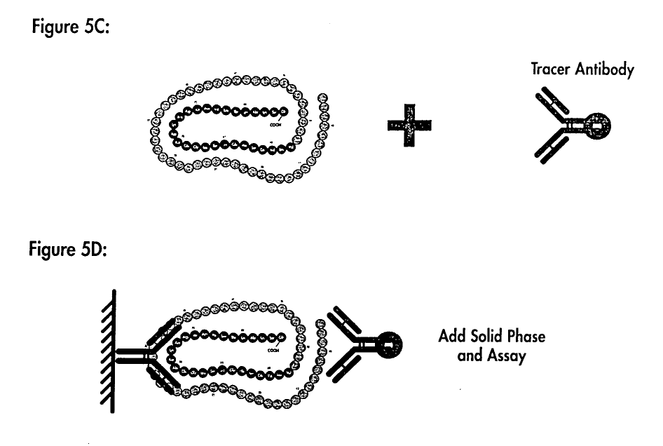

In leading to the present disclosure it was discovered that if interfering moieties can be targeted and bound by specific binding components that avoid binding analytes of interest, that these interfering moieties can be removed from a sample, thus permitting an assay of the sample that avoids cross-reactivity of both the analyte of interest and an interfering moiety with some homology to the analyte of interest with assay reagents. It was further discovered that the interfering moiety can be removed through causing it to become specifically bound to a particle such as a bead and removing the particle from the sample. In a frequent embodiment, the particle refers to an agarose gel. This embodiment requires the use of binding component reagents affixed to particles (including surfaces) that avoid binding the analyte of interest. In general, it was discovered in leading to the present disclosure that the use of a particle (bead) having a specific binding component affixed (e.g., covalently attached or attached by a non-covalent attachment) thereto allows the binding of specific undesired moieties or analytes in a sample and their associated removal from solution. In a frequent embodiment, this removal from solution can essentially remove the selected undesired moiety or analyte from being assayed. The remaining analyte of interest in the sample can then be assayed while avoiding cross-reactivity and potential assay inaccuracy typically caused by the now removed moiety or analyte. Although not intending to be bound by theory, insolubilisation refers to the binding of an analyte or moiety to a solid phase, and when an analyte/moiety has bound to one solid phase it cannot be bound to another solid phase of the present methods. An exemplary process utilizing analyte removal by insolubilization technology is depicted in FIG. 5.

In one embodiment, a sample is obtained from a subject, which sample may or may not contain an analyte of interest and/or an interfering moiety. The sample is then contacted with an isolation binding component to allow specific binding of the isolation binding component to the interfering moiety but not to the analyte, if the analyte and/or the interfering moiety is present in the sample, wherein the interfering moiety is removed from a solution phase in the sample upon binding with the isolation binding component. The isolation binding component is then permitted to specifically bind the interfering moiety. Although not bound by any particular theory, this binding causes the removal of the interfering moiety from a solution phase in the sample (or renders the interfering moiety insoluble), as the isolation binding component itself is or becomes insoluble in the sample. This insolubility is relative to the particular assay, and it is contemplated that exemplary isolation binding components may be utilized that are insoluble in the short term, but may become soluble. Such solubilization will generally occur after an assay is completed. On occasion, the sample and the isolation binding component are separated by removing the isolation binding component from the sample or removing the sample from the isolation binding component. Frequently, however, the isolation binding component remains in the sample until a subsequent washing step, prior to the detection step. In one embodiment, one or more multiple interfering moieties and one or more isolation binding components are contemplated.

The sample is then contacted with a tracer binding component such that the tracer binding component binds the analyte. This binding often, but not always, comprises specific binding. Before, concurrent therewith, or after binding of the tracer binding component, the sample is contacted with an assay solid phase binding component that specifically binds the analyte. The interfering moiety bound to the isolation binding component generally does not bind the assay solid phase binding component. In the wash step the interfering moiety bound to the isolation binding component is separated from the analyte of interest bound by the tracer binding component.

The analyte of interest bound by the tracer binding component is then detected by a method contemplated herein or otherwise known in the art. Frequently, if the tracer binding component is labeled, the assay used to detect the analyte of interest is dictated by the type of detectible label utilized. Often a two-step labeling process is undertaken. The means utilized to detect the labeled tracer binding component bound to the analyte of interest and/or the interfering moiety often generally comprises an assay of a format selected from the group consisting of, for example, an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, immuno radio metric assay (IRMA), μ-capture assay, linear flow membrane chromatography, inhibition assay, energy transfer assay, avidity assay, turbidometric immunoassay, and time resolved amplified cryptate emission (TRACE) assay.

The analyte is generally detected such that the presence, level and/or concentration is determined in the sample (and the subject). On occasion, the tracer binding component binds the interfering moiety, but is washed away prior to measurement or detection.

The isolation binding component often comprises a bead or other solid phase. Agarose beads (of a size frequently between about 4 to about 100 microns) are frequently used, especially beads of the type that are cyanogen bromide activated allowing covalent attachment of the binding component thereto. Although, any of the variety of solid phases discussed herein are contemplated. The isolation binding component is frequently capable of separation from the sample after the binding component positioned thereon binds the interfering moiety, even if the isolation binding component is not actually separated from the sample during the assay. If separation is desired, it may be in the form of simple removal from the sample. In addition, the sample can be removed from the isolation binding component. Frequently the isolation binding component is of the type that will settle to the bottom of an assay tube upon centrifugation, allowing the aspiration of the sample/supernatant from the tube for further assay. Often the isolation binding component is of the type that can be removed from solution through the application of an electric or magnetic field. On occasion, the isolation binding component is of a size that it will become entangled in a mesh or sieve or filter if the sample is passed through such a device, allowing the interfering moiety depleted sample to pass through. In a further occasional embodiment, a star tube (available from Nalge Nunc Int'l, Rochester, N.Y.) is utilized wherein the assay solid phase binding component is positioned in between 2 fins in the tube positioned above the floor of the tube such that precipitate could settle below the fin and the assay solid phase positioned thereon for example, a bead without being contaminated with the pelleted isolation binding component, which is bound to the analyte.

Once the interfering moiety becomes bound to the isolation binding component, the interfering moiety is essentially removed from solution and this removal from solution could be considered an insolubilization of the undesired analyte. This interfering moiety is then no longer eligible for assay as it is not part of the solution containing the analyte of interest. Often, when the interfering moiety is bound to the isolation binding component it cannot bind the assay solid phase, however, the tracer may occasionally bind thereto. But, as the sandwich is not completed with binding to the assay binding component, there will be no detection.

The assay binding component aspect of the isolation binding component is frequently specific for the interfering moiety such that it is capable of specifically binding the interfering moiety. In a frequent embodiment, multiple isolation binding components are employed, each having a binding component having a specificity for a different interfering moiety or a different region of the same interfering moiety. The binding component aspect of the isolation binding component is attached through any of a variety of means known or available in the art. Generally, the binding component is attached through means that do not detrimentally or fatally alter the capability of the binding component to bind its target ligand. For example, the binding component can be adsorbed to a particle. Other means of affixing the binding component comprise or involve carbodiimide, cyanogen bromide, passive adsorption, use of a second antibody, biotin and avidin coated solid phase, among others known in the art.

Solid phase technology is useful to remove interfering moieties from a sample. The specificity of the binding component aspect of the isolation binding component depends directly on the particular analyte of interest and potential interfering moieties in the sample. Generally, the isolation binding component is utilized to remove an interfering moiety from the sample. Often the isolation binding component is utilized to bind and hold the interfering moiety such that the analyte of interest can bind the assay solid phase and be transferred to a separate chamber/vessel for assay. Meanwhile, often the first reaction chamber/vessel is evaluated for the presence and/or level of the interfering moiety. Thus, although termed an “interfering” moiety, often this moiety comprises another analyte of interest.

In one example the isolation binding component can comprise an antibody having specificity for 1-3 PTH, 1-4 PTH, 1-5 PTH, 1-6 PTH, 1-7 PTH, 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 1-22 PTH, 1-23 PTH, 1-24 PTH, 1-25 PTH, 1-26 PTH, 1-27 PTH, 1-28 PTH, 1-29 PTH, 1-30 PTH, 1-31 PTH, 1-32 PTH, 1-33 PTH, 1-34 PTH, 2-5 PTH, 2-6 PTH, 2-7 PTH, 2-8 PTH, 2-9 PTH, 2-10 PTH, 2-11 PTH, 2-12 PTH, 2-13 PTH, 2-14 PTH, 2-15 PTH, 2-16 PTH, 2-17 PTH, 2-18 PTH, 2-19 PTH, 2-20 PTH, 2-21 PTH, 2-22 PTH, 2-23 PTH, 2-24 PTH, 2-25 PTH, 2-26 PTH, 2-27 PTH, 2-28 PTH, 2-29 PTH, 2-30 PTH, 2-31 PTH, 2-32 PTH, 2-33 PTH, 2-34 PTH, 3-6 PTH, 3-7 PTH, 3-8 PTH, 3-9 PTH, 3-10 PTH, 3-11 PTH, 3-12 PTH, 3-13 PTH, 3-14 PTH, 3-15 PTH, 3-16 PTH, 3-17 PTH, 3-18 PTH, 3-19 PTH, 3-20 PTH, 3-21 PTH, 3-22 PTH, 3-23 PTH, 3-24 PTH, 3-25 PTH, 3-26 PTH, 3-27 PTH, 3-28 PTH, 3-29 PTH, 3-30 PTH, 3-31 PTH, 3-32 PTH, 3-33 PTH, 3-34 PTH, 4-8 PTH, 4-9 PTH, 4-10 PTH, 4-11, PTH, 4-12 PTH, 4-13 PTH, 4-14 PTH, 4-15 PTH, 4-16 PTH, 4-17 PTH, 4-18 PTH, 4-19 PTH, 4-20 PTH, 4-21 PTH, 4-22 PTH, 4-23 PTH, 4-24 PTH, 4-25 PTH, 4-26 PTH, 4-27 PTH, 4-28 PTH, 4-29 PTH, 4-30 PTH, 4-31 PTH, 4-32 PTH, 4-33 PTH, 4-34 PTH, 5-9 PTH, 5-10 PTH, 5-11, PTH, 5-12 PTH, 5-13 PTH, 5-14 PTH, 5-15 PTH, 5-16 PTH, 5-17 PTH, 5-18 PTH, 5-19 PTH, 5-20 PTH, 5-21 PTH, 5-22 PTH, 5-23 PTH, 5-24 PTH, 5-25 PTH, 5-26 PTH, 5-27 PTH, 5-28 PTH, 5-29 PTH, 5-30 PTH, 5-31 PTH, 5-32 PTH, 5-33 PTH, 5-34 PTH, 6-9 PTH, 6-10 PTH, 6-11, PTH, 6-12 PTH, 6-13 PTH, 6-14 PTH, 6-15 PTH, 6-16 PTH, 6-17 PTH, 6-18 PTH, 6-19 PTH, 6-20 PTH, 6-21 PTH, 6-22 PTH, 6-23 PTH, 6-24 PTH, 6-25 PTH, 6-26 PTH, 6-27 PTH, 6-28 PTH, 6-29 PTH, 6-30 PTH, 6-31 PTH, 6-32 PTH, 6-33 PTH, 6-34 PTH, etc., among others. Although specificity up to position 34 is described above, the isolation binding component will, on occasion, have a specificity for positions beyond 34 on the PTH molecule. In addition, combinations of isolation binding components are contemplated comprising two or more binding components having varying, but complementary specificities for the PTH molecule in accordance with the present methods.

In a frequent example, the isolation binding component comprises a bead having an attached antibody specific for 1-9 PTH. As further described in the Examples, this bead may be contacted with a sample and then binds an interfering moiety comprising 1-84 PTH. The 1-84 PTH is then removed from solution. The remainder of the solution is then assayed by a traditional or known total PTH assay to determine the 7-84 PTH level. Frequently, the 7-84 PTH comprised in the remainder of the solution is transferred to another tube to be assayed. The 1-84 PTH that is removed from solution may also be assayed. Together, the assay values of 7-84 PTH and 1-84 PTH, optionally together with another PTH fragment, frequently yield a total PTH level.

Less frequently, a parallel sample is obtained from the subject which is reserved and not contacted with the bead. This sample may be assayed utilizing a traditional or known total PTH assay to determine the total PTH level. This total PTH level is then subtracted from the 7-84 PTH level (and/or another PTH fragment level, if determined) to determine the 1-84 PTH level in the subject.

The tracer binding component can have a specificity for an analyte of interest similar to that set forth above with regard to the Specificity by Blocking embodiment; however, it may have a broader specificity as the interfering moiety will occasionally be removed from the sample prior to introducing the tracer binding component. In a frequent embodiment, any binding component that can selectively bind the analyte of interest and can be detectibly labeled may be suitable. Also frequently, depending on the desired embodiment, any binding component that can bind the analyte of interest and/or the interfering moiety and can be detectibly labeled may be suitable.

In an occasional embodiment, the binding component aspect of the labeled tracer binding component binds the interfering moiety attached to the isolation binding component and/or the analyte attached to the assay solid phase. Specific binding pair members discussed herein and known in the art may be useful in such an embodiment to provide for such binding.

In an often included embodiment, the analyte of interest is present in the sample together with other moieties after removal of targeted interfering moieties from a solution phase. In this embodiment, these “other” moieties can be targeted for labeling with a tracer binding component, occasionally together with the analyte of interest. In one example, the analyte of interest is 7-84 PTH and the other moieties are PTH fragments other than 7-84 PTH. Moreover, in the example of PTH as the analyte of interest, the tracer binding component frequently has a specificity for a region on the N-terminal side of the PTH molecule (e.g., of the 7-84 PTH molecule). Frequently this region comprises one or more epitopes. This region on the N-terminal side of the PTH molecule is non-restrictive and generally refers to a region on the N-terminal side of position 34 of the PTH molecule. Nevertheless, on occasion, the tracer binding component is specific for a region in the mid-terminus or on the C-terminal side of the PTH molecule. In either case a two-step process is often utilized to avoid cross reactivity with circulating C-terminal PTH fragments other than 7-84 PTH.

The assay solid phase binding component may be similar to that described elsewhere herein. Features of the assay solid phase portion of this component are further described herein. Generally the specificity of the binding component aspect of the assay solid phase binding component will vary based on the desired analyte of interest such that it is structured to bind the analyte of interest. Often the assay solid phase binding component is specific for the analyte of interest. On occasion, the assay solid phase binding component would bind the interfering moiety, but for its removal from binding eligibility through binding the isolation binding component. Moreover, this specificity may also vary based on the specificity chosen for the isolation binding component. Frequently, the assay solid phase binding component specifically binds a region other than that bound by the isolation binding component and/or the labeled tracer binding component. For example, the analyte of interest can be a PTH molecule such as 7-84 PTH and the binding component aspect of the assay solid phase binding component can be an antibody having a specificity for 39-84 PTH. Frequently the binding component aspect of the assay solid phase binding component comprises an antibody specific for PTH, frequently falling within or comprising 39-84 PTH, a mid-terminal or C-terminal region of PTH. In an occasional embodiment, the specificity of the assay solid phase binding component and the labeled tracer binding component are reversed. Often, the analyte comprises an analyte other than PTH, such as calcitonin. The present methods can be adapted for use in evaluating the presence and/or level of calcitonin in a subject in the presence of procalcitonin or preprocalcitonin.

In one embodiment, the isolation binding component comprises a particle attached to a binding component. The particle frequently comprises a microtiter plate, a glass slide, a nitrocellulose membrane, cellulose or a cellulose derivative, a latex bead, a cell, an organelle, a protein or peptide, a test tube, a plastic bead, a colloidal gold particle, a colored particle, a magnetic bead, a quantum dot, a dipstick or a screen. Also frequently, the bead is comprised of cellulose or cellulose derivative, a polymer, latex, glass or metal. In a frequent embodiment, the bead is cyanogen bromide activated.

In another embodiment, the method steps take place in a reaction chamber. Frequently, the tracer binding component binds the interfering moiety and the analyte, the method further comprises detecting the binding between the tracer binding component and the interfering moiety. Also frequently, the detection of the binding between the analyte and the tracer binding component and the detection of the binding between the interfering moiety and the tracer binding component comprises determining the level of the analyte and the interfering moiety in the sample. Often, the analyte bound to the tracer binding component is removed from the reaction chamber into a second chamber prior to determining the level of the analyte and the interfering moiety in the sample. Relatedly, in another embodiment, the tracer binding component binds the interfering moiety, and the isolation binding component is removed from the reaction chamber after the binding component attached thereto binds the interfering moiety. This embodiment often further comprises detecting the level of interfering moiety after removal from the reaction chamber and detecting the level of analyte in the reaction chamber after removal of the interfering moiety therefrom. Often, the analyte of interest comprises 7-84 PTH or other PTH fragment(s) and the interfering moiety comprises 1-84 PTH, and the method optionally further comprises calculating a total PTH level from the combined levels of 7-84 PTH and 1-84 PTH.

Also frequently, a selection of two of the 7-84 PTH level, the total PTH level and/or the 1-84 PTH level are compared in a ratio. In addition to the levels of the PTH components in the sample, this ratio can be used to diagnose, monitor or guide treatment for a disease or disorder such as a renal bone disease of adynamic bone disease or high bone turnover disease. The gate, threshold and/or algorithm methods described herein may be utilized in the diagnosis, monitoring or guiding of treatment for a disease or disorder.

In another frequent embodiment, the binding component aspect of the isolation binding component, the tracer binding component, and/or the assay solid phase binding component comprises an antibody, an antibody fragment, a receptor, or a member of a specific binding pair. Frequently, each of these binding components may comprise a different type of binding component. For example, in one embodiment, the binding component aspect of the isolation binding component could be an antibody, the tracer binding component could comprise a member of a specific binding pair, and the assay solid phase binding component could comprise a receptor. In a frequent embodiment, when the binding component for each comprises an antibody, each antibody is a monoclonal or polyclonal antibody. Frequently, the binding component aspect of the isolation binding component, the labeled tracer binding component, and the assay solid phase binding component comprises an anti-PTH antibody.

As indicated, on occasion, the isolation binding component is removed from the sample after the binding component attached thereto binds the interfering moiety. This removal may occur prior to, concurrently therewith or after contacting the sample with the assay solid phase and/or the labeled tracer. Frequently, the tracer binding component binds the interfering moiety, and the isolation binding component is removed from the reaction chamber after the binding component attached thereto binds the interfering moiety. Also frequently, the level of interfering moiety is detected after removal from the reaction chamber and detecting the level of analyte of interest in the reaction chamber after removal of the interfering moiety therefrom.

2. Precipitating Reagent Technology

a. Cyclase Activating Parathyroid Hormone—Precipitating Removal (CAP-PR)

In another embodiment, methods are provided for improving the measurement of 7-84 PTH, 1-84 PTH and/or other PTH fragments by known assays. In this embodiment isolation binding components are provided that bind to the interfering moiety. In this embodiment precipitating reagents are provided that are capable of specifically binding indirectly to one or more particular PTH components that can lead to effecting their precipitation. The precipitation may be from the sample itself or a reaction solution, the reaction solution comprising a sample after one or more process steps. In a frequent embodiment, similar to the insolubulisation methodology set out above, a method for detecting an analyte in the presence of an interfering moiety is provided comprising: a) contacting a sample containing or suspected of containing an analyte and/or an interfering moiety with an isolation binding component to allow specific binding of the isolation binding component to the interfering moiety but not to the analyte, if the analyte and/or the interfering moiety is present in the sample, wherein the interfering moiety is removed from a solution phase in the sample by binding with the isolation binding component; b) contacting the sample with a tracer binding component to allow binding of the tracer binding component to the analyte; and d) detecting the binding between the analyte and the tracer binding component to assess the presence and/or amount of the analyte in the sample, wherein the analyte is a fragment, isoform or analog of the interfering moiety and step a) is conducted prior to step b). In a frequent embodiment, the method further comprises contacting the sample with an assay solid phase binding component to allow specific binding of assay binding component to the analyte but not to the interfering moiety.

Frequently, the interfering moiety is removed from solution via the formation of an interfering moiety complex that is formed upon the contact of the sample with a complex forming binding component capable of binding with the isolation binding component. Also frequently, the method further comprises contacting the sample with a nonspecific immunoglobulin composition that is derived from the same species as the isolation binding component, wherein the complex forming binding component is further capable of binding the nonspecific immunoglobulin composition to further form the interfering moiety complex which comes out of solution via precipitation. Also frequently, the isolation binding component comprises a mouse derived monoclonal antibody composition, the nonspecific immunoglobulin composition comprises mouse immunoglobulin, and the second immunoglobulin composition comprises goat anti-mouse immunoglobulin. On occasion, the nonspecific immunoglobulin is not derived from the same species as the isolation binding component, and the complex forming binding component optionally comprises a composition wherein at least one binding component of the composition is also capable of preferentially binding the nonspecific immunoglobulin.

In an exemplary embodiment a method is provided for direct measurement of 7-84 PTH (and/or another PTH fragment) in the presence of 1-84 PTH, 7-84 PTH and/or another PTH fragment. In this exemplary embodiment, reagents comprising anti-1-9 PTH antibody (e.g., mouse anti-1-9 PTH monoclonal antibody) and mouse immunoglobulin are introduced to a sample. The anti-1-9 PTH antibody then binds 1-84 PTH but not 7-84 PTH present in the sample during incubation. The mouse immunoglobulin does not bind either analyte. After the mouse anti-1-9 PTH antibody has had sufficient time to bind the 1-84 PTH goat anti-mouse immunoglobulin is then introduced to the sample which acts as a precipitating antibody. This antibody is not species-limited, but will often be of a type that will bind the mouse immunoglobulin reagent and/or antibody present in the sample. Similarly, the mouse immunoglobulin and anti-1-9 PTH antibody reagent could be replaced by non-mouse versions, with the associated change of the specificity of the precipitating antibody. For example, guinea pig anti-1-9 PTH or rabbit anti-1-9 PTH could be utilized, with the corresponding use of guinea pig immunoglobulin or rabbit immunoglobulin as carrier(s), and thus goat anti-guinea pig immunoglobulin or goat anti-rabbit immunoglobulin are contemplated. The goat anti-mouse immunoglobulin binds to the mouse 1-9 PTH antibody (which is bound to 1-84 PTH) and to the mouse immunoglobulin, thus forming an immune complex that can precipitate out of solution. This precipitation renders 1-84 PTH insoluble and not capable of efficient binding to the assay solid phase binding component which is necessary for detection in a total PTH assay. Meanwhile, 7-84 PTH is not bound the precipitating antibody and remains in solution and remains in a soluble state where it can be measured by the PTH assay. The 1-84 PTH which has been bound by the 1-9 PTH antibody and made insoluble as a precipitated immune complex is removed from the assay solid phase binding component during the wash phase prior to detecting the label on the assay solid phase binding component.

Thereafter the sample is assayed for 7-84 PTH and/or another PTH fragment. In a frequent embodiment, a total PTH assay (or reagents useful therefore) is then utilized on the sample. This assay may or may not be undertaken without removing the 1-84 PTH precipitated (insoluble) mass, however the 1-84 PTH precipitated (insoluble) mass is removed from the presence of the assay solid phase binding component during the wash stage prior to label detection. 7-84 PTH is then detected by the total PTH assay. This step may be performed using any of a variety of total PTH assays that incorporate reagents that have the ability to bind soluble 7-84 PTH and/or 1-84 PTH. The present embodiment allows for the direct measurement of 7-84 PTH and/or other PTH fragments, without using the above mentioned subtraction method. In a less occasional embodiment, two parallel samples can be assayed at the same time or in sequence, one utilizing the reagents and methods described above, and the other without these reagents, but according to the total PTH assay instructions. The first of these will yield a 7-84 PTH level determination and the second will yield a total PTH level determination. One could determine the 1-84 PTH level by subtracting the 7-84 PTH level, and/or other PTH fragments levels, from the total PTH level.

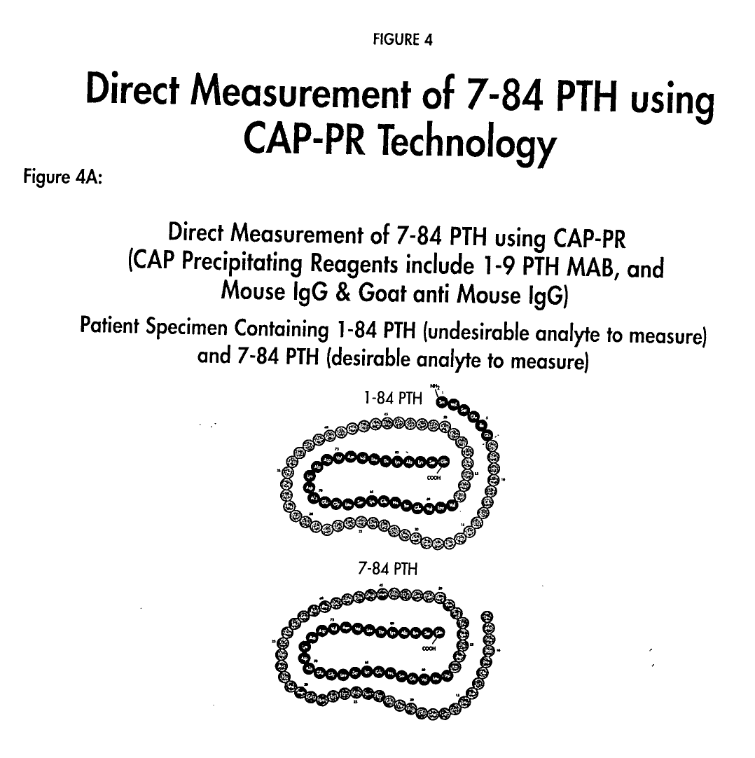

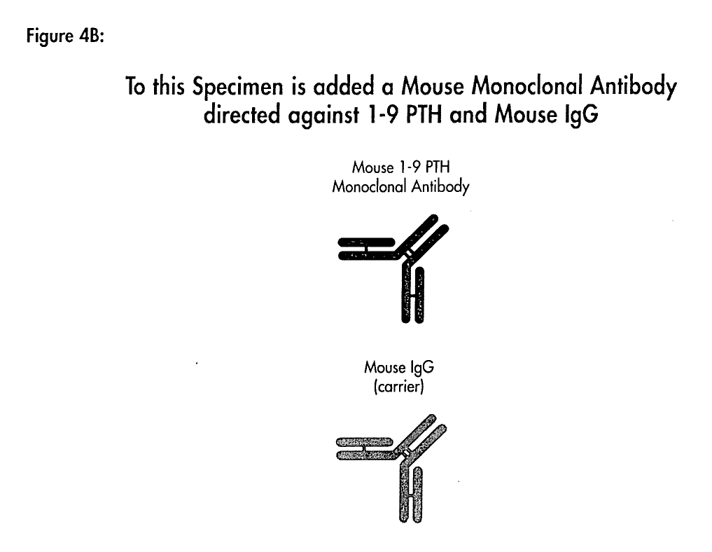

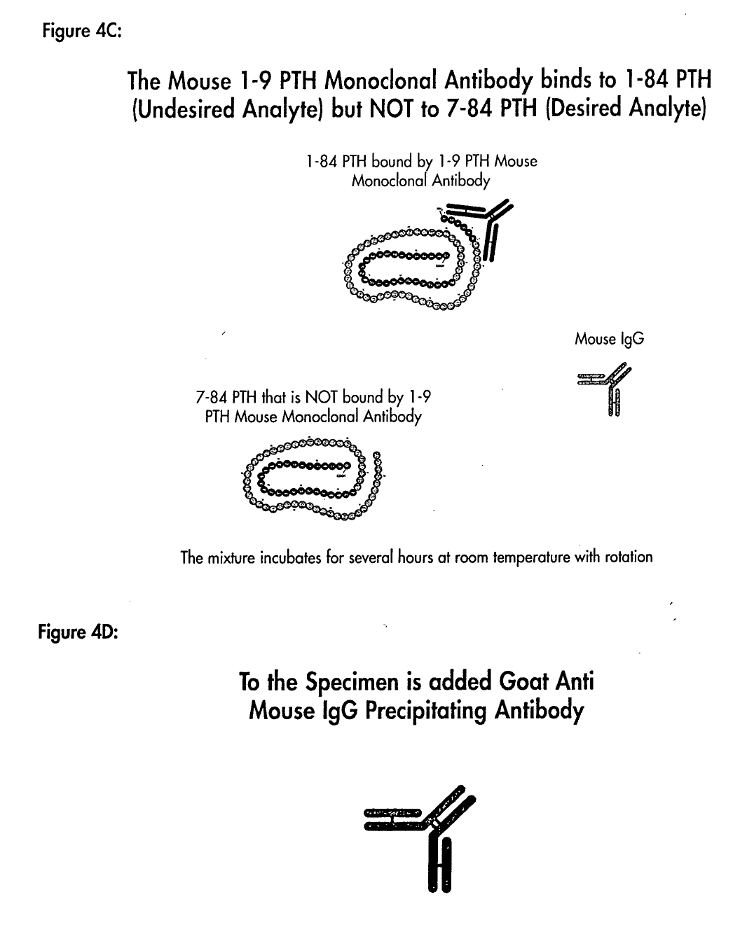

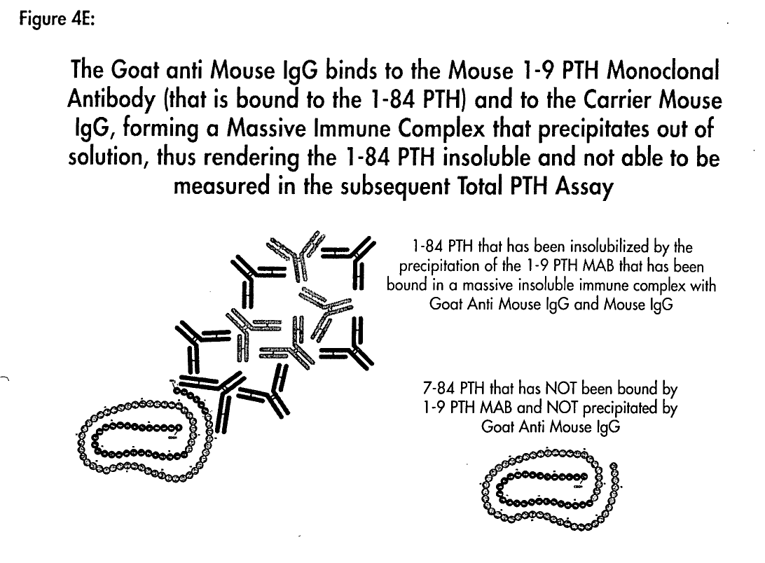

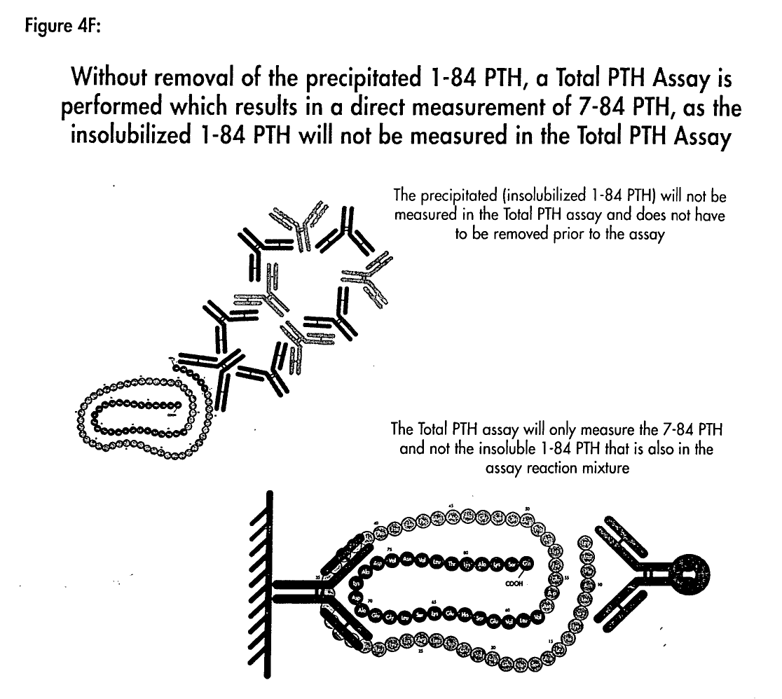

FIG. 4 provides a depiction of an exemplary method utilizing CAP-PR technology using precipitating antibodies. In this example, a 1-9 PTH mouse monoclonal antibody along with mouse immunoglobulin both of which are not attached to a solid phase are added to a sample from a subject, which is then incubated for several hours (e.g., about 5-10 hours) at room temperature with rotation. The 1-9 mouse antibody then binds the 1-84 PTH present in the sample (4C). Following the incubation, goat anti mouse immunoglobulin (precipitating antibody) is added to the specimen which causes a precipitation of the monoclonal antibody bound to the 1-84 PTH as well as a precipitation of the mouse immunoglobulin, but not binding nor causing a precipitation of 7-84 PTH and/or another PTH fragment (4D-4E). The goat anti mouse immunoglobulin binds to the mouse 1-9 PTH monoclonal antibody (that is bound to the 1-84 PTH) and to the carrier mouse immunoglobulin, forming a massive immune complex that precipitates out of solution, thus rendering the 1-84 PTH insoluble and not able to be measured in the subsequent total PTH assay. 1-84 PTH that has been insolubilized by the precipitation of the 1-9 PTH antibody that has been bound in a massive insoluble immune complex with goat anti mouse immunoglobulin and mouse immunoglobulin. 7-84 PTH that has not been bound by 1-9 PTH antibody and not precipitated by goat anti mouse immunoglobulin is measured. The total PTH assay capture bead and tracer is then added, without the separation of the precipitate, and the assay proceeds as a normal total PTH assay (4F). The precipitated (insolubilized 1-84 PTH) will not be measured in the total PTH assay and does not have to be removed prior to the assay. The total PTH assay will only measure the 7-84 PTH and not the insoluble 1-84 PTH that is also in the assay reaction mixture. During the wash stage the precipitate is often removed from the presence of the total PTH assay capture bead. The result is direct a measurement of 7-84 PTH (and/or another PTH fragment) without measuring 1-84 PTH.

b. Whole PTH Measurement

In another embodiment utilizing precipitating reagent technology a method is provided for measuring whole PTH or 1-84 PTH. In sum, this embodiment provides the precipitation and measurement of whole PTH. An advantage is provided through the practice of the present methods as no solid phase reagents are necessary for the performance of the assay. This provides an advantage in the form of alleviating any steric hindrance of the binding of the analyte with the assay binding component that may occasionally be present in assay binding components affixed to a solid phase. Moreover, solid phase reagents are often costly and time intensive to produce. In addition, solid phase reagents are often instable and/or bulky and generally require higher binding component concentrations on a per test basis to ensure effective assay performance. Thus, the use of liquid reagents will decrease the overall cost of assay production and practice.

Accordingly, in one embodiment, a method is provided for detecting whole PTH in a sample comprising: a) contacting a fluid sample containing or suspected of containing whole PTH and/or PTH fragments with a tracer binding component to allow specific binding of the tracer binding component to the whole PTH; b) contacting the sample with an isolation binding component to allow specific binding of the isolation binding component to the whole PTH; c) optionally contacting the sample with a nonspecific binding component, wherein the nonspecific binding component is derived from the same species as the isolation binding component; d) contacting the sample with a complex forming binding component to allow binding of the complex forming binding component to the isolation binding component bound to the whole PTH and the nonspecific binding component to form a complex, wherein the complex precipitates out of solution; and e) detecting the binding between the tracer binding component and the whole PTH, wherein any or all of steps a), b) or c) are conducted prior to step d). Frequently sample comprises whole PTH in addition to PTH fragments and the method allows for selective detection of the whole PTH.

Frequently, the method further comprises the removal of unbound tracer binding component, unprecipitated isolation binding component, unprecipitated nonspecific binding component and/or unprecipitated complex forming binding component, if any, prior to the detection of the binding between the tracer binding component and the whole PTH. Often this removal is accomplished through one or more wash steps, wherein the sample, containing the assay reagents, is washed through the introduction of a wash reagent and centrifuged (or otherwise separated) to pellet the complex containing the whole PTH. Often, the complex forming binding component is contacted with the sample in a composition that acts as a wash reagent. Thus, frequently the unbound tracer binding component, unprecipitated isolation binding component, unprecipitated nonspecific binding component, unprecipitated complex forming binding component and/or other sample components such as PTH fragments are comprised in a supernatant removed during the wash and discarding of the supernatant from the pelleted complex.

In a frequent embodiment, tracer binding component and isolation binding component comprise antibodies or fragments thereof. Often one or both of these antibodies comprise monoclonal antibodies. In another frequent embodiment, the nonspecific binding component comprises a nonspecific immunoglobulin, and the complex forming binding component comprises another immunoglobulin molecule that is capable of binding the nonspecific immunoglobulin. In one embodiment, the tracer binding component comprises an anti-1-9 PTH antibody. In another embodiment, the isolation binding component comprises an anti-39-84 PTH antibody. Frequently, the tracer binding component comprises an antibody that is derived from a different species than the isolation antibody. In a frequent embodiment, the tracer binding component comprises a goat anti-1-9 PTH antibody, the isolation binding component comprises a mouse anti-39-84 PTH antibody, the nonspecific binding component comprises a mouse immunoglobulin, and the complex forming binding component comprises a goat anti-mouse immunoglobulin. Also frequently, the nonspecific binding component comprises rabbit immunoglobulin, the isolation binding component comprises rabbit anti-39-84 PTH antibody and the complex forming binding component comprises goat anti-rabbit immunoglobulin. Thus, the reagents may be derived from any of a variety of species known in the art, so long at the general binding specificities of the present method are maintained. On occasion, the nonspecific immunoglobulin is not derived from the same species as the isolation binding component, and the complex forming binding component optionally comprises a composition wherein at least one binding component of the composition is also capable of preferentially binding the nonspecific immunoglobulin.

Similar to other embodiments described herein, the tracer binding component frequently further comprises a label selected from the group consisting of an enzyme and a substrate, a chromogen, a catalyst, a chemiluminescent compound, a particulate label, a fluorescent label, an enzymatic label, a colorimetric label, a dye label, a radioactive label, and a magnetic label. Often the label comprises 125-Iodine.

Although whole PTH is specifically exemplified in the present methods, other analytes described herein can be utilized in accordance with the present methods.

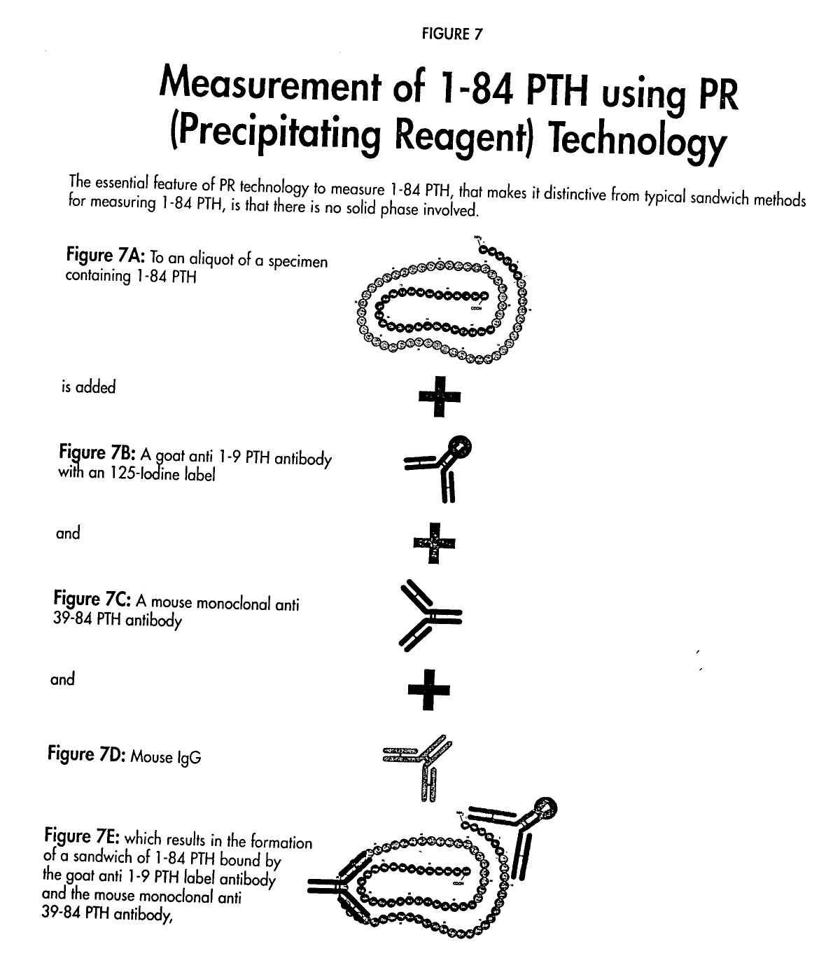

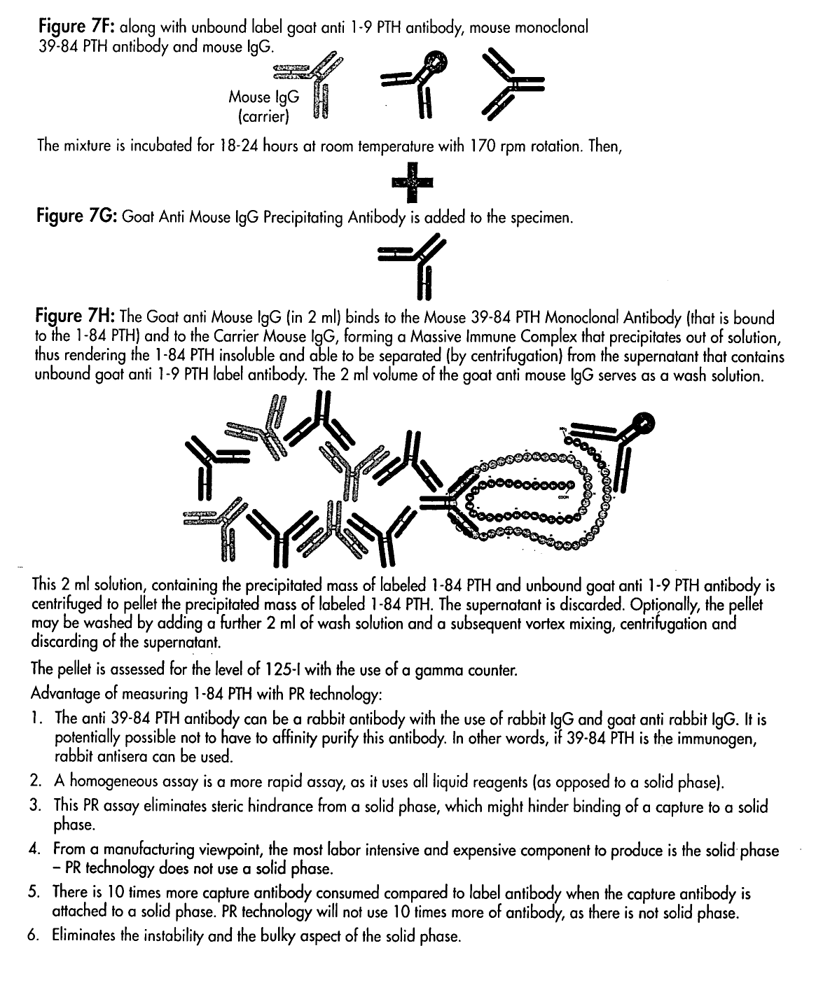

FIG. 7 provides an exemplary depiction of the present embodiment.

c. Calcitonin Measurement

Calcitonin is a protein that plays an important role in calcium metabolism in humans, fish and rodents. Therefore, there is significant clinical utility in measuring and monitoring the presence and levels of this protein in subjects. Calcitonin comprises a 32 amino acid protein that is formed by the production and processing of precursor proteins preprocalcitonin and procalcitonin. Procalcitonin is a protein of 116 amino acids in length. This protein is degraded into two larger polypeptides corresponding to amino acids 1-57 (N-procalcitonin-(1-57)-peptide) and 60-116 (C-procalcitonin-(60-116)-peptide) prior to the production of calcitonin. The latter of these two polypeptides is split into the polypeptides calcitonin and katacalcin. Calcitonin, therefore, represents a degradation product of procalcitonin that corresponds to amino acids 60-91 of procalcitonin.

Procalcitonin (human) comprises a peptide having the following sequence:

Ala-Pro-Phe-Arg-Ser-Ala-Leu-Glu-Ser-Ser-Pro-Ala-
Asp-Pro-Ala-Thr-Leu-Ser-Glu-Asp-Glu-Ala-Arg-Leu-
Leu-Leu-Ala-Ala-Leu-Val-Gln-Asp-Tyr-Val-Gln-Met-
Lys-Ala-Ser-Glu-Leu-Glu-Gln-Glu-Gln-Glu-Arg-Glu-
Gly-Ser-Ser-Leu-Asp-Ser-Pro-Arg-Ser-Lys-Arg-Cys-
Gly-Asn-Leu-Ser-Thr-Cys-Met-Leu-Gly-Thr-Tyr-Thr-
Gln-Asp-Phe-Asn-Lys-Phe-His-Thr-Phe-Pro-Gln-Thr-
Ala-Ile-Gly-Val-Gly-Ala-Pro-Gly-Lys-Lys-Arg-Asp-
Met-Ser-Ser-Asp-Leu-Glu-Arg-Asp-His-Arg-Pro-His-
Val-Ser-Met-Pro-Gln-Asn-Ala-Asn

Calcitonin (human) comprises a peptide having the following sequence:

Cys-Gly-Asn-Leu-Ser-Thr-Cys-Met-Leu-Gly-Thr-Tyr-
Thr-Gln-Asp-Phe-Asn-Lys-Phe-His-Thr-Phe-Pro-Gln-
Thr-Ala-Ile-Gly-Val-Gly-Ala-Pro

The Calcitonin C-Terminal Flanking Peptide (human) comprises:

Asp-Met-Ser-Ser-Asp-Leu-Glu-Arg-Asp-His-Arg-Pro-
His-Val-Ser-Met-Pro-Gln-Asn-Ala-Asn
(C-Procalcitonin (human); Katacalcin; PDN-21)

The Calcitonin N-Terminal Flanking Peptide (human) comprises:

Ala-Pro-Phe-Arg-Ser-Ala-Leu-Glu-Ser-Ser-Pro-Ala-
Asp-Pro-Ala-Thr-Leu-Ser-Glu-Asp-Glu-Ala-Arg-Leu-
Leu-Leu-Ala-Ala-Leu-Val-Gln-Asp-Tyr-Val-Gln-Met-
Lys-Ala-Ser-Glu-Leu-Glu-Gln-Glu-Gln-Glu-Arg-Glu-
Gly-Ser-Ser-Leu-Asp-Ser-Pro-Arg-Ser

Thus, the calcitonin protein is 32 amino acids in length. See, e.g., H. D. Niall, et al., Biochemistry (1969) 64:771-8 (describing various species forms of calcitonin). Assays used to measure calcitonin often cross-react with procalcitonin or preprocalcitonin as they contain overlapping, homologous sequences.

Accordingly, a method is provided for detecting calcitonin in the presence of an interfering moiety comprising a calcitonin precursor in a sample comprising: a) contacting a sample containing or suspected of containing calcitonin and/or a calcitonin precursor with an isolation binding component to allow specific binding of the isolation binding component to the interfering moiety but not to the calcitonin, if the analyte and/or the interfering moiety is present in the sample, wherein the interfering moiety is removed from a solution phase in the sample by binding with the isolation binding component; b) contacting the sample with a tracer binding component to allow binding of the tracer binding component to the analyte; and c) detecting the binding between the analyte and the tracer binding component to assess the presence and/or amount of the analyte in the sample, wherein the analyte is a fragment or isoform of the interfering moiety and step a) is conducted prior to step b). In general, the isolation binding component binds to an epitope on the interfering moiety that is not an epitope found on calcitonin, in whole or in part. Frequently, the interfering moiety is removed from solution via the formation of an interfering moiety complex that is formed upon the contact of the sample with a complex forming binding component capable of binding with the isolation binding component.

In an often included embodiment, the method further comprises contacting the sample with a nonspecific immunoglobulin composition that is derived from the same species as the isolation binding component, wherein the complex forming binding component is further capable of binding the nonspecific immunoglobulin composition to further form the interfering moiety complex. Also frequently, the isolation binding component comprises a mouse derived monoclonal antibody composition, the nonspecific immunoglobulin composition comprises mouse immunoglobulin, and the second immunoglobulin composition comprises goat anti-mouse immunoglobulin. In accordance with the present methods, often, the interfering moiety comprises procalcitonin or preprocalcitonin. On occasion, the nonspecific immunoglobulin is not derived from the same species as the isolation binding component, and the complex forming binding component optionally comprises a composition wherein at least one binding component of the composition is also capable of preferentially binding the nonspecific immunoglobulin.

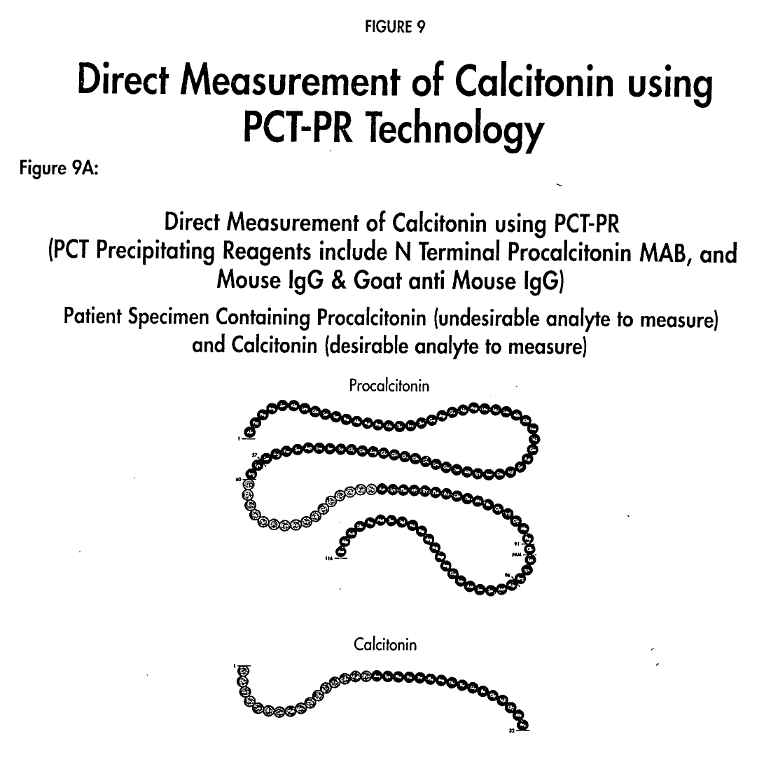

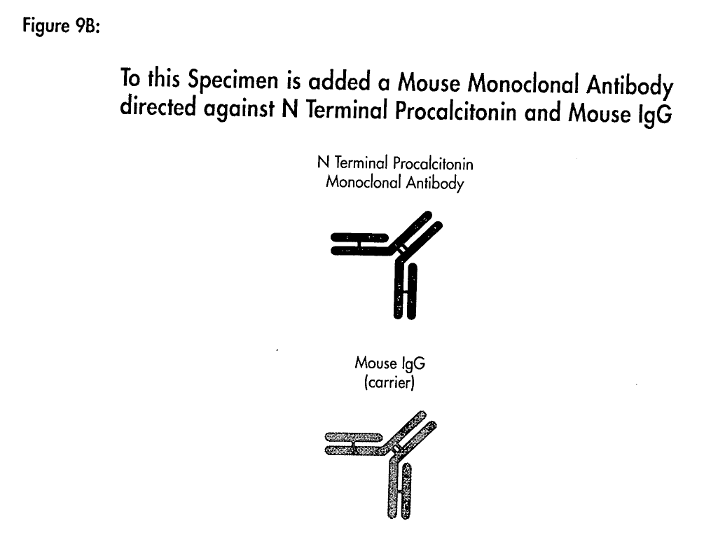

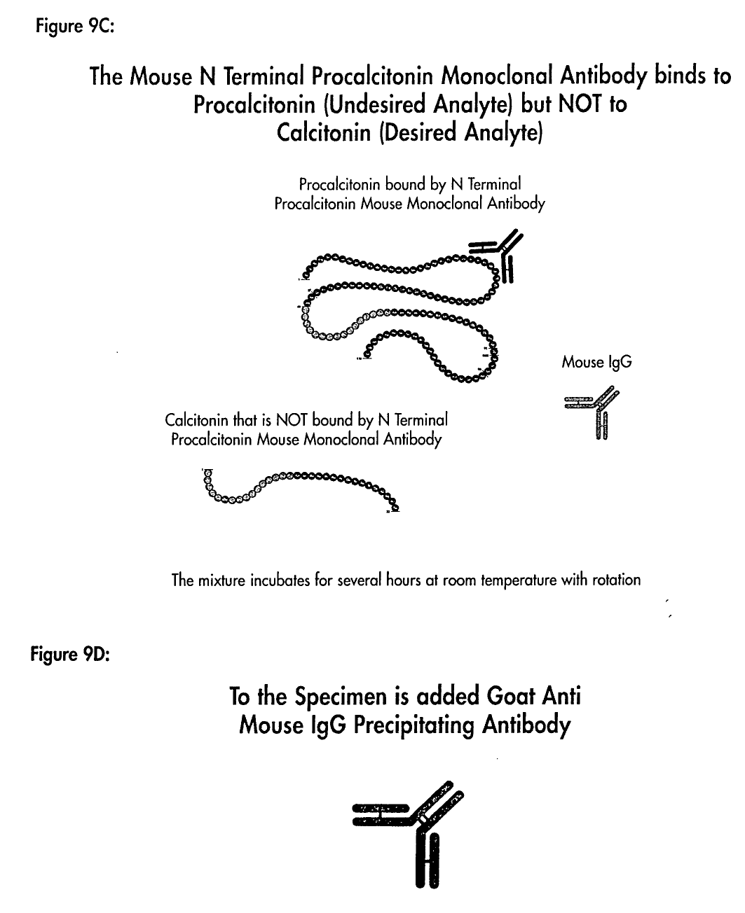

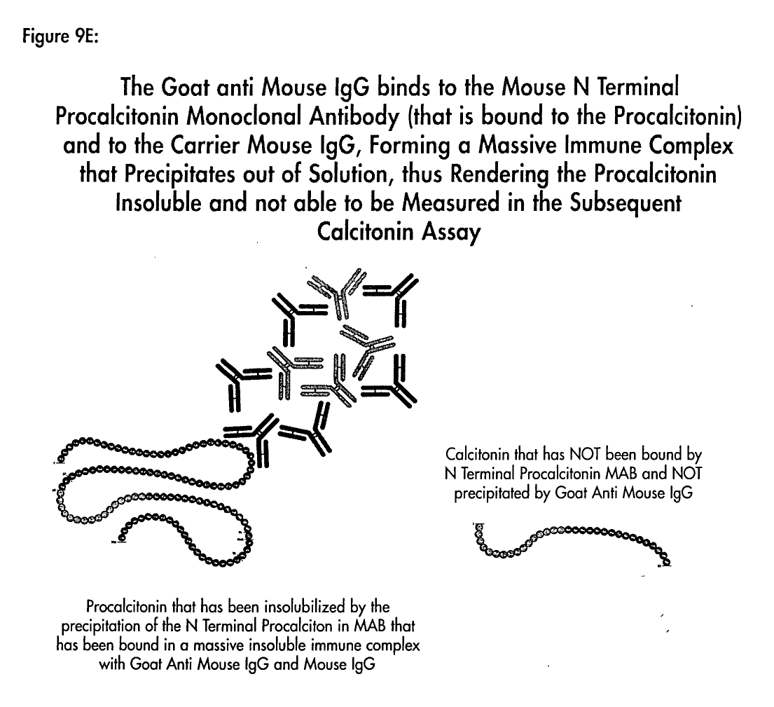

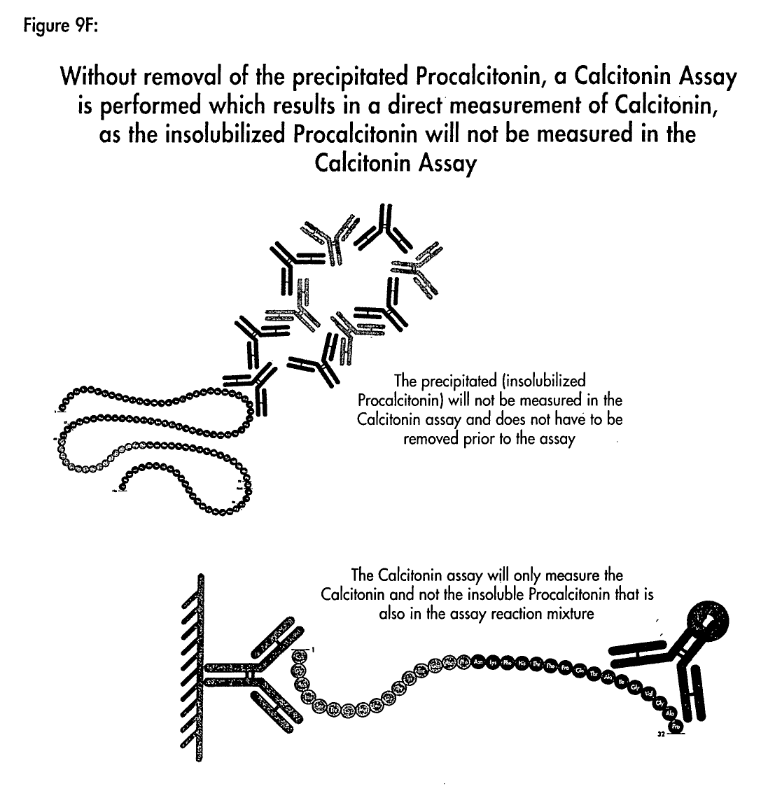

FIG. 9 provides a depiction of an exemplary method utilizing HCT-PR technology using precipitating antibodies. In this example, a procalcitonin mouse monoclonal antibody along with mouse immunoglobulin (both of which are not attached to a solid phase) are added to a sample from a subject, which is then incubated for several hours (e.g., about 5-10 hours) at room temperature with rotation. The procalcitonin mouse antibody then binds the procalcitonin present in the sample (9C). Following the incubation, goat anti mouse immunoglobulin (precipitating antibody) is added to the specimen which causes a precipitation of the monoclonal antibody bound to the procalcitonin as well as a precipitation of the mouse immunoglobulin, but not binding nor causing a precipitation of calcitonin (9E). The goat anti mouse immunoglobulin binds to the mouse procalcitonin monoclonal antibody (that is bound to the procalcitonin) and to the carrier mouse immunoglobulin, forming a massive immune complex that precipitates out of solution, thus rendering the procaltonin insoluble, and frequently not able to be measured. Procalcitonin that has been insolubilized by the precipitation of the procalcitonin antibody bound in an insoluble immune complex with goat anti mouse immunoglobulin and mouse immunoglobulin precipitates out of solution. Calcitonin that has not been bound by procalcitonin antibody and not precipitated by goat anti mouse immunoglobulin remains in solution.

Then, the calcitonin assay capture bead and tracer are added, without the separation of the precipitate (9F). The precipitated (insolubilized procalcitonin) will not be measured in the calcitonin assay and does not have to be removed prior to the assay. The calcitonin assay will only measure the calcitonin and not the insoluble procalcitonin that is also in the assay reaction mixture. During the wash stage the precipitate is removed from the presence of the calcitonin assay capture bead. The assay capture bead with the calcitonin and label antibody can now be read to measure calcitonin. The result is direct a measurement of calcitonin without measuring procalcitonin. Although the depicted example removes the precipitated procalcitonin, such removal is not a required aspect of the present methods.

On occasion, a method is provided for measuring procalcitonin in accordance with other methods set forth herein. As indicated elsewhere, procalcitonin comprises an analyte of interest contemplated in the present disclosure.

d. Trio Kit Technology

In another embodiment, a method is provided for conducting an evaluation for multiple analytes and/or interfering moieties in a single sample. This method is useful to determine the presence and/or level of two or three or more analytes of interest. In an exemplary embodiment, the method is directed to determining PTH levels in a sample. Often this method is utilized to determine the total PTH level, the 1-84 PTH level, the 7-84 PTH level and/or another PTH fragment level in a single sample. Frequently, only a single sample of about 200 microliters is required, although all sample volumes are contemplated. As indicated elsewhere herein, a sample is frequently a blood or serum sample.

In a frequent embodiment, a method is provided for detecting an analyte in the presence of an interfering moiety comprising: a) placing a sample containing or suspected of containing an analyte and an interfering moiety in a reaction chamber; b) contacting the sample with an isolation binding component, wherein the isolation binding component specifically binds the interfering moiety but not the analyte in the sample; c) contacting the sample with a tracer binding component that binds the analyte and the interfering moiety in the sample; d) contacting the sample with an assay solid phase binding component that binds with the analyte in the sample; and e) selectively detecting the binding between the tracer binding component and: (i) the analyte, (ii) the interfering moiety, and/or (iii) the combination of the analyte and the interfering moiety, wherein the analyte is a fragment, analog or isoform of the interfering moiety, wherein step b) is conducted prior to steps c) and/or d), and wherein the assay solid phase binding component bound to the analyte and/or the isolation binding component bound to the interfering moiety are optionally removed from the reaction chamber and into another chamber prior to selective detection of (i) the analyte or (ii) the interfering moiety.

In one example the isolation binding component can comprise an antibody having specificity for 1-3 PTH, 1-4 PTH, 1-5 PTH, 1-6 PTH, 1-7 PTH, 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 1-22 PTH, 1-23 PTH, 1-24 PTH, 1-25 PTH, 1-26 PTH, 1-27 PTH, 1-28 PTH, 1-29 PTH, 1-30 PTH, 1-31 PTH, 1-32 PTH, 1-33 PTH, 1-34 PTH, 2-5 PTH, 2-6 PTH, 2-7 PTH, 2-8 PTH, 2-9 PTH, 2-10 PTH, 2-11 PTH, 2-12 PTH, 2-13 PTH, 2-14 PTH, 2-15 PTH, 2-16 PTH, 2-17 PTH, 2-18 PTH, 2-19 PTH, 2-20 PTH, 2-21 PTH, 2-22 PTH, 2-23 PTH, 2-24 PTH, 2-25 PTH, 2-26 PTH, 2-27 PTH, 2-28 PTH, 2-29 PTH, 2-30 PTH, 2-31 PTH, 2-32 PTH, 2-33 PTH, 2-34 PTH, 3-6 PTH, 3-7 PTH, 3-8 PTH, 3-9 PTH, 3-10 PTH, 3-11 PTH, 3-12 PTH, 3-13 PTH, 3-14 PTH, 3-15 PTH, 3-16 PTH, 3-17 PTH, 3-18 PTH, 3-19 PTH, 3-20 PTH, 3-21 PTH, 3-22 PTH, 3-23 PTH, 3-24 PTH, 3-25 PTH, 3-26 PTH, 3-27 PTH, 3-28 PTH, 3-29 PTH, 3-30 PTH, 3-31 PTH, 3-32 PTH, 3-33 PTH, 3-34 PTH, 4-8 PTH, 4-9 PTH, 4-10 PTH, 4-11, PTH, 4-12 PTH, 4-13 PTH, 4-14 PTH, 4-15 PTH, 4-16 PTH, 4-17 PTH, 4-18 PTH, 4-19 PTH, 4-20 PTH, 4-21 PTH, 4-22 PTH, 4-23 PTH, 4-24 PTH, 4-25 PTH, 4-26 PTH, 4-27 PTH, 4-28 PTH, 4-29 PTH, 4-30 PTH, 4-31 PTH, 4-32 PTH, 4-33 PTH, 4-34 PTH, 5-9 PTH, 5-10 PTH, 5-11, PTH, 5-12 PTH, 5-13 PTH, 5-14 PTH, 5-15 PTH, 5-16 PTH, 5-17 PTH, 5-18 PTH, 5-19 PTH, 5-20 PTH, 5-21 PTH, 5-22 PTH, 5-23 PTH, 5-24 PTH, 5-25 PTH, 5-26 PTH, 5-27 PTH, 5-28 PTH, 5-29 PTH, 5-30 PTH, 5-31 PTH, 5-32 PTH, 5-33 PTH, 5-34 PTH, 6-9 PTH, 6-10 PTH, 6-11, PTH, 6-12 PTH, 6-13 PTH, 6-14 PTH, 6-15 PTH, 6-16 PTH, 6-17 PTH, 6-18 PTH, 6-19 PTH, 6-20 PTH, 6-21 PTH, 6-22 PTH, 6-23 PTH, 6-24 PTH, 6-25 PTH, 6-26 PTH, 6-27 PTH, 6-28 PTH, 6-29 PTH, 6-30 PTH, 6-31 PTH, 6-32 PTH, 6-33 PTH, 6-34 PTH, etc., among others. Although specificity up to position 34 is described above, the isolation binding component will, on occasion, have a specificity for positions beyond 34 on the PTH molecule. In addition, combinations of isolation binding components are contemplated comprising two or more binding components having varying, but complementary specificities for the PTH molecule in accordance with the present methods.

The tracer binding component may comprise any of a variety of specificities for a PTH molecule. Generally, however, the tracer binding component is capable of binding 1-84 and/or 7-84 PTH. Often the tracer binding component is capable of binding one or more PTH fragments in addition to, or other than, 7-84 PTH. Thus, the tracer binding component may be of a specificity that can bind these PTH molecules, specifically or otherwise. Frequently, the tracer binding component is of a specificity that can bind 1-84 PTH and/or 7-84 PTH when either or both of these molecules are bound by other binding components described herein. The tracer binding component frequently further comprises a detectible label as contemplated herein.

The tracer binding component may be comprised of more than one different tracer binding component. The tracer binding components may be added in one step or they may be added in different steps. Therefore, frequently, the binding component aspect of the reagents of a particular assay of the present description are compatible based on the structure and/or sequence of the analyte of interest, and often the interfering moiety. In an occasional embodiment, the binding component aspect of the labeled tracer binding component binds the isolation binding component and/or the assay solid phase bound to the interfering moiety and analyte of interest, respectively. Specific binding pair members discussed herein and known in the art may be useful in such an embodiment to provide for such binding.

In an often included embodiment, the present method comprises introducing a sample containing or suspected of containing both 1-84 PTH, 7-84 PTH (and/or another PTH fragment) to a tube (reaction chamber). The tube comprises walls coated with a specific isolation binding component, occasionally referred to as a capture antibody. In a frequent embodiment, the capture antibody coated on the tube comprises anti-1-9 PTH antibody (e.g., goat anti-1-9 PTH) or another antibody that can selectively or specifically bind 1-84 PTH without binding 7-84 PTH. 1-84 PTH present in the sample then binds the coated tube wall, but 7-84 PTH is not bound by the 1-84 PTH capture antibody on the walls of the tube. In practice, the tube may be incubated with the sample for a period of time, e.g., 1-12 hours, about 18-24 hours, or about 6 hours, at a specific temperature, e.g., room temperature. Often the tube will be rotated on an orbital mixer during incubation, e.g., at about 170 rotations per minute. After incubation, a bead coated with another anti-PTH antibody is introduced to the tube. Frequently the antibody attached to the bead can selectively or specifically bind 7-84 PTH. In one example, the antibody on the bead can comprise a specificity for a region comprising 39-84 of the PTH molecule (see, e.g., U.S. Pat. No. 6,689,566), although other specificities are contemplated. The “bead” may comprise a collection of beads and the “antibody” may comprise a collection of antibodies. In a frequent embodiment, this antibody bound bead comprises an assay solid phase. Although not intending to be bound by theory, 1-84 PTH bound to the wall of the tube or chamber avoids binding the antibody attached to the bead. Moreover, the bead assay binding component may not bind the 1-84 PTH bound to the wall of the tube or chamber. A tracer binding component can be added to the sample which is capable of binding both of 1-84 PTH and 7-84 PTH. Frequently the binding component aspect of the tracer binding component comprises an antibody or antibody fragment. On occasion, the tracer can comprise two or more antibodies so that both the 1-84 PTH and the 7-84 PTH become bound to the tracer binding component. These two or more antibodies may be added together or separately. An incubation step is provided to permit binding of the tracer binding component(s) to 1-84 PTH and 7-84 PTH. Frequently the tracer binding component will be specific for a region or epitope on PTH that is not already bound by the isolation binding component (i.e., capture antibody on the tube wall) and is also not bound by the bead assay solid phase binding component (i.e., capture antibody on the bead surface). Based on the above example, an anti-7-34 PTH tracer antibody would be suitable.

After tube and bead antibody binding and tracer binding component binding, the tube and bead may be washed together as per normal assay procedures for washing and, if the tracer binding component is labeled, analyzed for label corresponding to total PTH levels comprising 1-84 PTH and 7-84 PTH levels. The bead and tube need not be washed together. As both components (analyte of interest and interfering moiety) are labeled and the bead is inside the tube, the analysis of both the labeled tube and the labeled bead will comprise counting the total concentration of labeled analyte or interfering moiety in the tube.

In addition, the assay solid phase is frequently separated into another tube or means for separate analysis. In the above example, this separate analysis is useful to determine the 7-84 PTH level (and/or another PTH fragment) in the sample as the assay solid phase is bound to 7-84 PTH. Once the assay solid phase is removed from the first tube (by transferring to the bead to another tube that was not used in the assay), this first tube (assay tube or reaction chamber) can be analyzed for 1-84 PTH levels. The 7-84 PTH concentration (determined from detecting label on the assay solid phase after separation from the antibody coated tube) and the 1-84 PTH concentration (determined from detecting label on the tube after separation from the bead) can be added together to calculate the total PTH concentration in the sample. Together the three assays directly measure total PTH, 1-84 PTH, 7-84 PTH levels (and/or another PTH fragment) in the sample. In a less frequent embodiment, depending on the specificity of the reagents and the presence of PTH fragments other than 7-84 PTH, a calculation method may be utilized to determine PTH levels such that when the results of two of the analytes comprising total PTH, 1-84 PTH and 7-84 PTH are known, they may be calculated to determine the level of the third analyte or the level of the third analyte (e.g., 1-34 PTH), together with other components. As indicated, together with other embodiments described herein, the mode utilized to determine the level of the analyte of interest in the sample depends in large part on the type of label utilized. In the example of the use of radioactive label (e.g., 125-Iodine), the tubes are counted for radioactivity (CPM) using a gamma counter of known means.

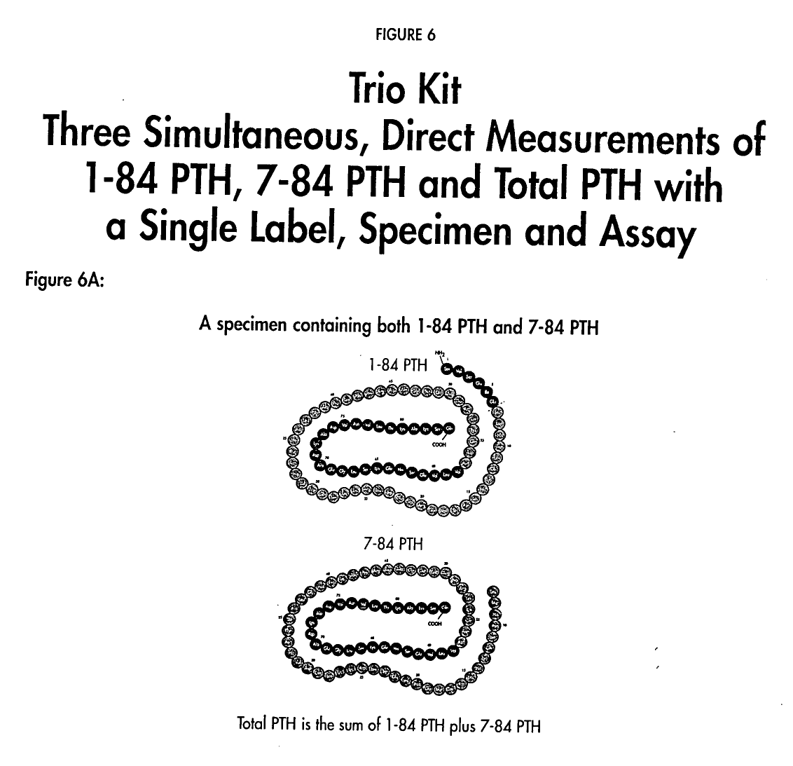

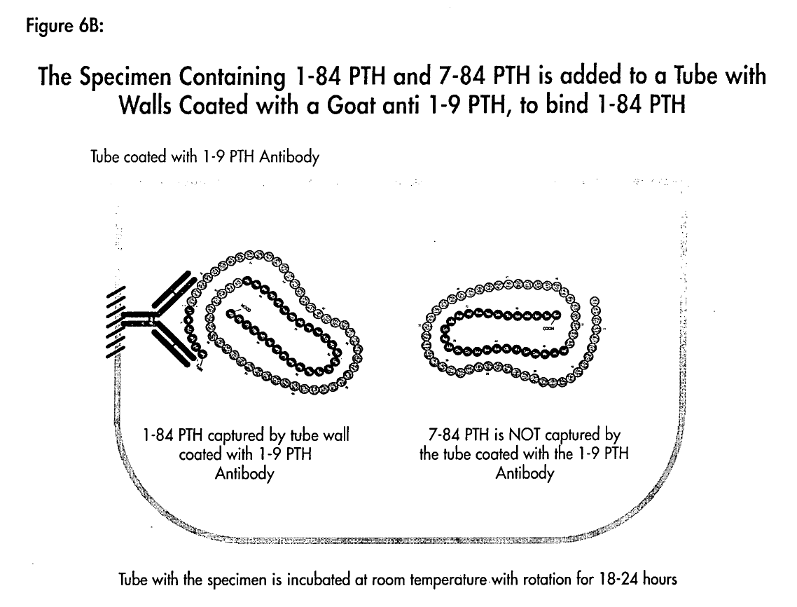

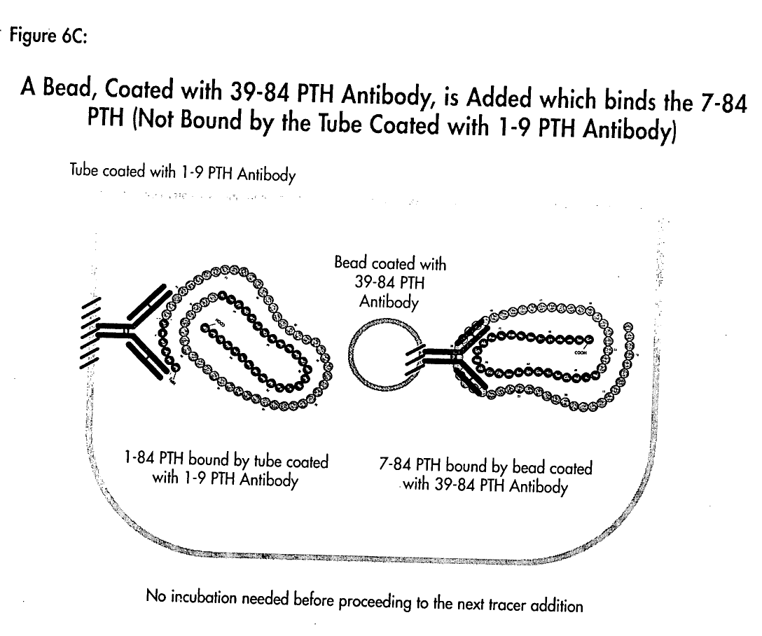

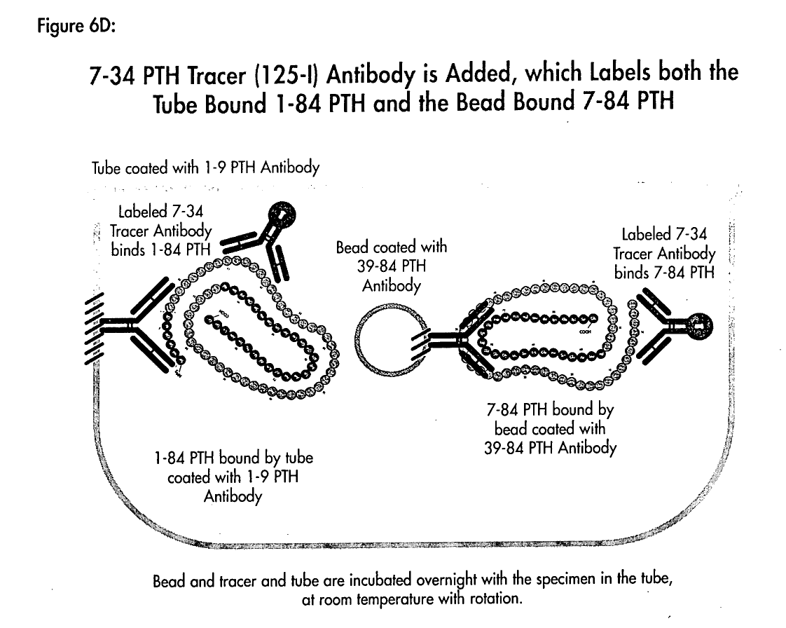

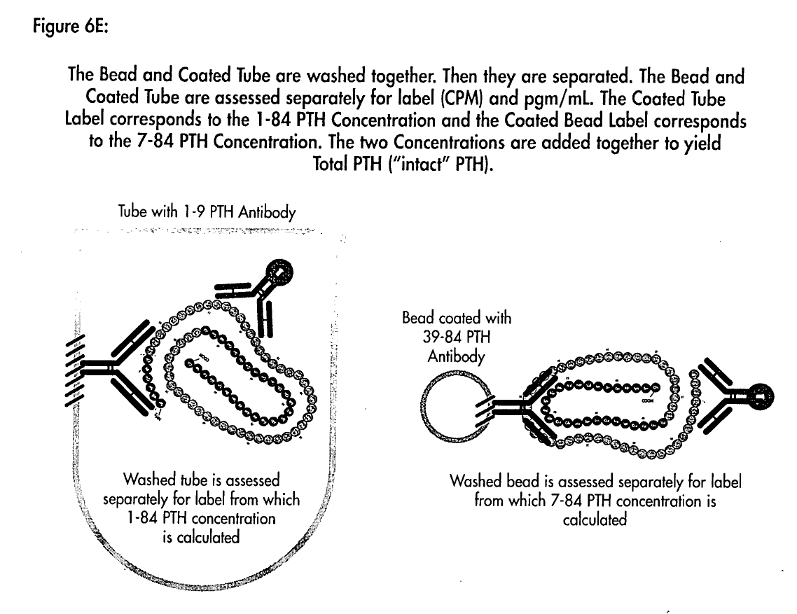

An exemplary process utilizing trio kit technology is depicted in FIG. 6(A-E). In this embodiment, the specimen containing 1-84 PTH and 7-84 PTH is added to a tube with walls coated with a goat anti 1-9 PTH, to bind 1-84 PTH, and is then incubated for hours (e.g., about 18-24 hours) (6A-6B). 1-84 PTH is captured by tube wall coated with 1-9 PTH antibody, but 7-84 PTH is not captured by the tube coated with the 1-9 PTH antibody. A bead, coated with 39-84 PTH antibody, is added which binds the 7-84 PTH, but is not bound by the tube coated with 1-9 PTH antibody (6C). An incubation is not necessary in this step. Then, 7-34 PTH tracer (125-I) antibody is added, which labels both the tube bound 1-84 PTH and the bead bound 7-84 PTH (6D). Often, the bead and tracer and tube are incubated overnight with the specimen in the tube, at room temperature with rotation. Then, the bead and coated tube are washed together, then separated. The bead and coated tube are assessed separately for label (CPM) and pgm/mL (6E). The coated tube label corresponds to the 1-84 PTH concentration and the coated bead label corresponds to the 7-84 PTH concentration. The two concentrations are added together to yield a total PTH (“intact” PTH) level.

In an alternative embodiment, a test sample, e.g., 300 microliters of plasma sample, can be incubated with a tube and a bead both coated with an anti-N-terminus PTH antibody, e.g., 1-9 PTH antibody, for a certain time, e.g., 5 hours, at an appropriate temperature, e.g., room temperature. After the incubation, a portion of the liquid, e.g., 200 microliters, can be transferred to a new container, e.g., a test tube, for measuring 7-84 PTH. The 7-84 PTH, which is now essentially free from 1-84 PTH and other PTH fragments with an intact N-terminus, can be measured by any suitable methods, e.g., using a 39-84 PTH antibody coated bead and 7-34 PTH tracer antibody. The 1-84 PTH and other PTH fragments with an intact N-terminus now attached to both the tube and a bead coated with 1-9 PTH antibody can be measured by any suitable methods, e.g., using a 39-84 PTH tracer antibody for 1-84 PTH and other appropriate tracer antibody for PTH fragments with an intact N-terminus. The 1-84 PTH and other PTH fragments with an intact N-terminus can be measured while bound to the original tube and/bead, or can be released from the tube or bead before the measurement. Similarly, the 1-84 PTH and other PTH fragments with an intact N-terminus can be measured in the original tube, or be transferred to a new container before the measurement. When the 1-84 PTH and other PTH fragments with an intact N-terminus are measured in the original tube, the residue liquid may be kept in the original tube or may be removed from the tube before the measurement. The 1-84 PTH, other PTH fragments with an intact N-terminus and the 7-84 PTH can be added together to yield a total PTH (“intact” PTH) level.

In another occasional embodiment, an additional solid phase particle having a binding component attached thereto is introduced to the sample. This additional particle is labeled with a label that is distinguishable from the label on the tracer binding component, if labeled. Generally, this additional solid phase particle is specific for a different region on the interfering moiety and/or the analyte and will bind fragments other than the analyte of interest. For example, where the interfering moiety comprises 1-84 PTH and the analyte comprises 7-84 PTH, the additional solid phase particle may be specific for an epitope or region comprising 39-65 PTH. This region, of course, may vary depending on the desired specificity. Methods provided herein provide for the production of binding components having the desired specificity. The additional solid phase particle is introduced to the sample after the sample has incubated with the isolation and assay solid phase and/or tracer binding components.

In an alternative embodiment, the coated tube can be replaced by the use of another substrate having the same reagent attached thereto. This substrate is preferably selectively distinguishable from the bead described above. For example, in an exemplary process a sample can be obtained and placed in a tube or reaction column/chamber, such as those available from Pierce Biotechnology (Rockford, Ill.). In a frequent embodiment, a gel having anti-1-9 PTH bound thereto is introduced to the column and the sample, wherein 1-84 PTH present in the sample is allowed to bind the gel bound antibody. A bead having a capture antibody such as anti-39-84 PTH is then introduced to the reaction column. In addition, a tracer antibody in accordance with the above description is introduced to the sample. The tracer antibody and the bead bound capture antibody are then permitted to bind with available analyte. After the first incubation of the sample and the gel affixed to the 1-9 PTH antibody, the 1-84 PTH binds to the gel. Thereafter the bead coated with the 39-84 PTH antibody (e.g., the assay solid phase) is added and the second incubation is started during which time the 7-84 PTH becomes bound to the bead. Then the 7-34 PTH tracer is added that binds both the 1-84 PTH, which is bound to the gel, and the 7-84 PTH, which is bound to the bead, and an incubation proceeds. After this incubation, an end wash solution can be added to the column, the column is centrifuged and the supernatant is aspirated. This wash step may be repeated one or more times. Thereafter the column containing the gel and the bead (and the bound labeled analyte of interest and interfering moiety) are counted and then the bead is transferred to another tube and the columns having the gel and the bead are evaluated/counted. The evaluation detects the presence, level and/or concentration of the analyte of interest, the interfering moiety and/or a combination of the two.

As another example, the substrate could comprise a magnetic particle or other substrate that can be distinguished or removed from the sample or reaction solution containing the bead attached to the anti-39-84 PTH antibody, which is bound to 7-84 PTH. This embodiment is similar to the above method, except that instead of centrifugation, the tube is placed on a magnet to pellet by magnetization the 1-9 PTH antibody coated magnetic particles bound with 1-84 PTH. The supernatant is then aspirated and discarded. This step may be repeated one or more times.

In one embodiment, the analyte and the interfering moiety are present in the reaction chamber upon selective detection of the binding between the tracer binding component and the combination of the analyte and the interfering moiety. Often, however, the analyte is removed from the reaction chamber after the sample is contacted with the isolation binding component. Frequently, the analyte is contacted with the tracer binding component and/or the assay solid phase binding component either (i) on contact with the other reaction chamber, or (ii) subsequent to removal to the other reaction chamber.

In another embodiment, the isolation binding component is attached to a wall of the reaction chamber such that upon placing at least a portion of the sample in the reaction chamber, the sample contacts the isolation binding component.

In a frequent embodiment, the tracer binding component is labeled with a detectable label and the tracer binding component comprises a first labeled tracer binding component that specifically binds the analyte and a second labeled tracer binding component that specifically binds the interfering moiety, wherein the label aspect of the first labeled tracer binding component is detectably distinguishable from the label aspect of the second labeled tracer binding component.

In a further embodiment, the labeled tracer binding component comprises a first labeled tracer binding component that specifically binds the analyte of interest and a second labeled binding component that specifically binds the interfering moiety, wherein the label aspect of the first labeled tracer binding component is detectably distinguishable from the label aspect of the second labeled tracer binding component.

In one embodiment, the method comprises detecting the analyte comprises determining the level of 7-84 PTH in the sample and detecting the interfering moiety comprises determining the level of 1-84 PTH in the sample. Frequently, the method further comprises calculating a total PTH level from the combined levels of 7-84 PTH and 1-84 PTH.

In another embodiment, detecting the combination of labeled analyte and interfering moiety in the sample comprises detecting the total PTH level in the sample, and wherein the 7-84 PTH level is subtracted from the total PTH level to determine the level of 1-84 PTH in the sample.

In a further embodiment, the detection of the analyte comprises determining the level of 7-84 PTH in the sample and the detection of the interfering moiety comprises determining the level of 1-84 PTH in the sample, wherein the analyte is detected in the other reaction chamber, and the interfering moiety is detected in the reaction chamber

Often, a selection of two of the 7-84 PTH level, the total PTH level and the 1-84 PTH level are compared in a ratio. The levels and/or the ratio can be used to diagnose, monitor or guide treatment for a disease or disorder. The gate, threshold and/or algorithm methods described herein may be utilized in the diagnosis, monitoring or guiding of treatment for a disease or disorder.

Often the disease or disorder is selected from the group consisting of osteoporosis, kidney stone disease, familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, Graves disease, the extent of parathyroid gland surgical removal, oversuppression with vitamin D or a vitamin D analogue or a calcimimetic or calcium, and chronic uremia.

In a frequent embodiment, the binding component aspect of each of the isolation binding component, the tracer binding component, and the assay solid phase binding component comprises an antibody, an antibody fragment or a member of a specific binding pair. Also frequently, the binding component aspect of each of the isolation binding component, the tracer binding component, and the assay solid phase binding component comprises a monoclonal or polyclonal antibody.

In an often included embodiment, the assay solid phase binding component bound to the analyte is removed from the reaction chamber and moved into another chamber prior to selective detection of the analyte or the interfering moiety, and the binding between the tracer binding component and the analyte and the binding between the tracer binding component and the interfering moiety are selectively determined, wherein such determination comprises determining the level of the analyte and the level of the interfering moiety in the sample. Often, the analyte comprises 7-84 PTH and the interfering moiety comprises 1-84 PTH and 1-34 PTH, wherein the method further comprises determining the level of 1-84 PTH in the sample by a direct 1-84 PTH assay. In this embodiment, the assay is often undertaken with the methods provided herein but with an additional assay of the sample utilizing an assay capable of specifically detecting 1-84 PTH, such as the CAP PTH assay (available from Scantibodies Laboratories, Inc., Santee, Calif.). The 1-84 PTH level determined utilizing the trio kit technology discussed herein is then subtracted from the 1-84 PTH level determined through the practice of the specific 1-84 PTH assay to determine the presence, concentration or level of 1-34 PTH in the sample. This embodiment is often useful for a subject when they are receiving administration of a PTH therapeutic such as teraparatide (e.g., FORTEO® (available from Eli Lilly, Indianapolis, Ind.)).

Often, the analyte comprises an analyte other than PTH, such as calcitonin. The present methods can be adapted for use in evaluating the presence and/or level of calcitonin in a subject in the presence of procalcitonin or preprocalcitonin.

e. PTH Drug Monitoring Kit

1-34 PTH is a drug known as teriparatide or FORTEO® and is often administered for treatment of osteoporosis. As teriparatide is a synthetic form of PTH (or a PTH analog) it is intended to mimic the biological effects of PTH in many ways, most notably on the rate of bone turnover. Accordingly, it is desirable to monitor the level of both 1-34 PTH (or teriparatide) and 1-84 PTH in subjects. The present embodiments can accomplish this goal utilizing a single patient specimen. The present embodiments are also useful for detecting medium to larger N-terminal PTH fragments, if present.

Accordingly, in a frequent embodiment a method is provided for detecting PTH and fragments or analogs thereof in a sample comprising: a) contacting a sample containing or suspected of containing whole PTH and an N-terminal PTH fragment or analog with an isolation binding component to allow specific binding of the isolation binding component to the whole PTH but not to the N-terminal PTH fragment or analog; b) contacting the sample with an assay solid phase binding component to allow specific binding of the assay solid phase binding component to the N-terminal PTH fragment or analog but not to the whole PTH; c) contacting the sample with a tracer binding component to allow binding of the tracer binding component to the whole PTH and the N-terminal PTH fragment or analog; and d) detecting the binding between the tracer binding component and the whole PTH, the N-terminal PTH fragment or analog and/or the combination of the whole PTH and the N-terminal PTH fragment or analog, wherein step a) is conducted prior to steps b) and/or c). As indicated, frequently the N-terminal PTH fragment or analog comprises 1-34 PTH or teraparatide.

In an often included embodiment, the sample is contacted with the isolation binding component in a reaction vessel, and the isolation binding component is immobilized on a surface of the reaction vessel. On occasion, however, the isolation binding component is immobilized on a solid phase other than a surface of the reaction vessel. Moreover, as referred to herein, “surface” is intended in its broadest sense and includes the outermost face of a solid phase, as well as a region below the outermost face of a solid phase (e.g., within the solid phase matrix), and a region above the outermost face of a solid phase (e.g., if reagents such as linkers and coatings are utilized).

Frequently, the isolation binding component comprises an anti-39-84 PTH antibody and wherein the isolation binding component binds a C-terminal PTH fragment, if present, in addition to binding whole PTH. Often, the tracer binding component does not bind the C-terminal fragment.

In one embodiment, the isolation binding component comprises an anti-39-84 PTH antibody, the assay solid phase binding component comprises an anti-15-34 PTH antibody, and the assay tracer comprises an anti-1-9 PTH antibody.

Frequently, the assay solid phase bound to the 1-34 PTH or teriparatide is removed from the reaction vessel into another vessel for detection. Often, the binding between the whole PTH and the tracer binding component is detected in the reaction vessel after the assay solid phase bound to the 1-34 PTH or teriparatide is removed.

In another frequent embodiment, the method further comprises contacting a second (or another) tracer binding component with the sample after contacting the sample with the assay solid phase binding component to allow binding of the second tracer binding component to a PTH fragment comprising 7-84 PTH, if present, in the sample, and detecting the binding between the second tracer binding component and the 7-84 PTH. Often, the second tracer binding component comprises an anti-15-34 PTH antibody.

Also frequently, the detection of the binding between the whole PTH and the tracer binding component comprises determining the level of whole PTH in the sample, wherein the detection of the binding between the 1-34 PTH or teriparatide and the tracer binding component comprises determining the level of 1-34 PTH or teriparatide in the sample, and wherein a total whole PTH and 1-34 PTH or teriparatide level is calculated from the level of whole PTH and the level of 1-34 or teriparatide.

In a further related embodiment, the method further comprises measuring or calculating the level of a PTH fragment comprising 7-84 PTH in the sample. Frequently, the 7-84 PTH level is measured utilizing methods described herein. Often, however, the 7-84 PTH level is measured or calculated utilizing the subtraction method described herein and known in the art. When measured, frequently the level of 1-34 PTH or teriparatide, whole PTH, 7-84 PTH, and/or combinations or ratios generated therefrom are utilized to diagnose, monitor or guide treatment for a disease or disorder. The gate, threshold and/or algorithm methods described herein may be utilized in the diagnosis, monitoring or guiding of treatment for a disease or disorder. The disease or disorder is generally one of the type described elsewhere herein. Frequently, the disease or disorder comprises osteoporosis.

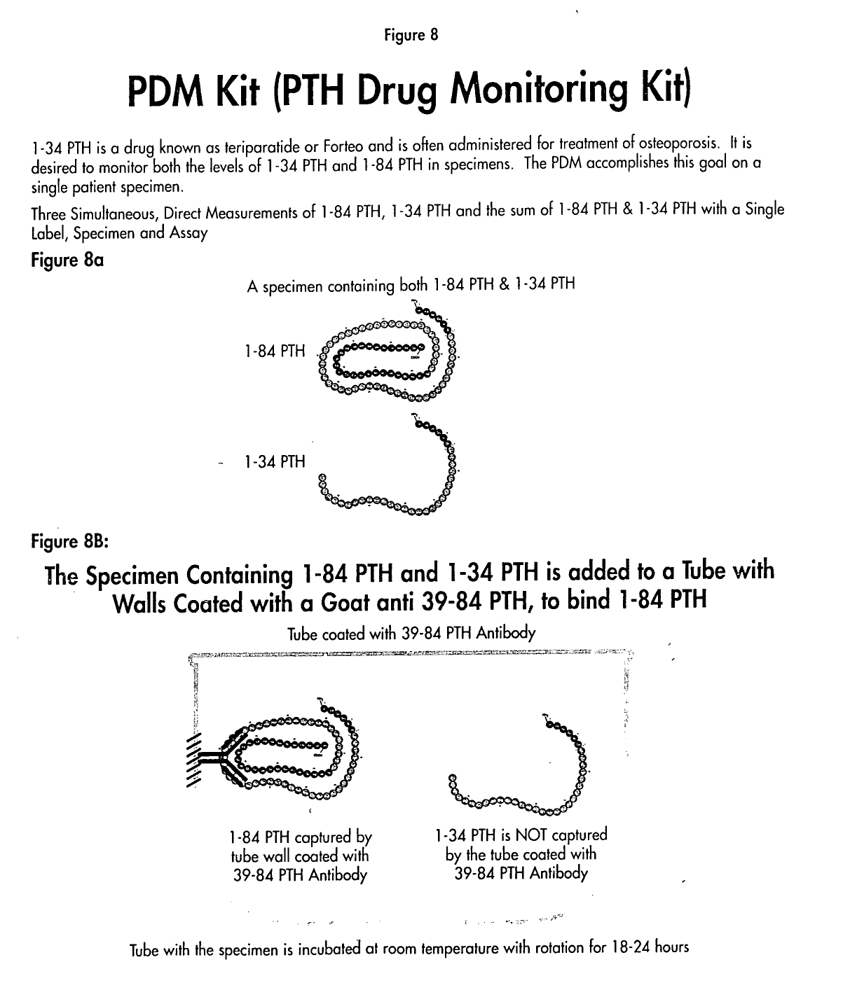

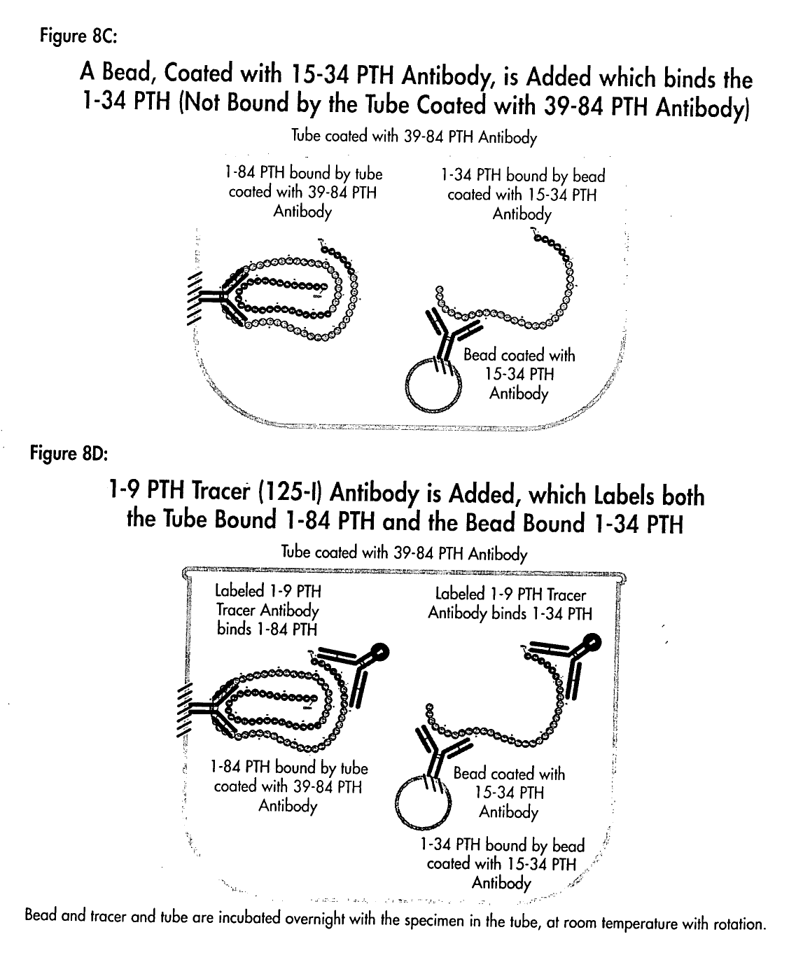

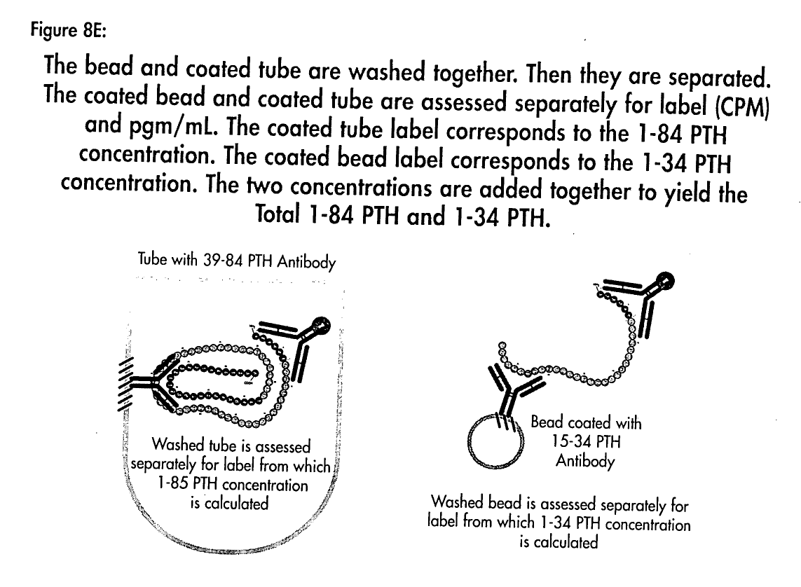

FIG. 8 provides an exemplary description of the present embodiments. As depicted therein, a sample containing or suspected of containing 1-84 PTH and/or 1-34 PTH is obtained from a subject. This sample is added to a tube having walls coated with a goat anti-39-84 PTH antibody (8A). 1-84 PTH present in the sample then binds with the goat anti-39-84 PTH antibody and therefore, the tube wall (8B). 1-34 PTH present in the sample does not bind with the goat anti-39-84 PTH antibody as the target epitope is not present (8B). The sample is then permitted to incubate in the tube, with rotation, for about 18-24 hours. Thereafter, a bead, coated with anti-15-34 PTH antibody is introduced to the sample in the tube (8C). This antibody coated bead binds with the 1-34 PTH present in the sample. Although not intending to be bound by theory, the anti-15-34 PTH antibody does not bind 1-84 PTH, if present, in the sample as the 1-84 PTH is already bound to the goat anti-39-84 PTH antibody. An anti-1-9 PTH tracer antibody is also added to the sample (8D). This tracer antibody is labeled with a 125-Iodine label and binds both the 1-84 PTH and 1-34 PTH present in the sample. This combination is then incubated, with rotation, for several hours. The bead and the coated tube are then washed together, but then separated (8E). Once separated, the bead and tube are assessed separately for label (CPM) and pgm/mL. The coated tube label corresponds to the 1-84 PTH concentration and the coated bead label corresponds to the 1-34 PTH concentration. The two concentrations can then be added together to obtain a total 1-84 PTH and 1-34 PTH level. On occasion, the bead and tube are assessed prior to separation.

f. Antibody Preparation

Proteins and peptides useful for antibody generation can be produced by a variety of methods known in the art. For example, such proteins and peptides may be produced by conventional methods including solid-phase peptide synthesis, see, e.g., R. B. Merrifield, et al., Biochemistry 21:5020 (1982), solution phase peptide synthesis or by recombinant technology. For related methods see U.S. patent application Ser. No. 10/799,476, filed Mar. 11, 2004. Thus, such peptides or proteins can be isolated or synthetically/recombinantly produced by methods known in the art.

Polyclonal antibodies can be produced in vivo in response to immunization antigens (e.g., proteins, peptides, haptens, chemical compounds, etc.). Anti-serum can be raised in a variety of animals and monitored via an ELISA assay (or other assays known in the art). Often, an antigen comprising a small molecule or a hapten, is coupled to a carrier to induce an immunological reaction. Monoclonal antibodies provide single epitope specificity and a large volume of identical antibody. In contrast, polyclonal antibodies often provide multiple specificities and are occasionally limited in volume, in part, due to the amount of serum that can be obtained from an immunized animal. However, the specificity of polyclonal antibodies can be improved by affinity chromatography using purified or synthetic antigen (the volume can be larger if an animal such as a goat is used which has the advantage of providing more serum volume than smaller animals such as the guinea pig or rabbit).

Synthetic short peptides are frequently utilized to generate antibodies. This approach involves synthesizing short peptide sequences, often then coupling them to a large carrier molecule, and immunizing the animal of choice with the peptide-carrier molecule.

In one exemplary embodiment, a method of producing a specific binding component/antibody is as follows: Human 1-84 PTH is obtained or synthesized comprising an amino acid sequence:
SVSEIQLMHN LGKHLNSMER VEWLRKKLQD VHNFVALGAP LAPRDAGSQR PRKKEDNVLV ESHEKSLGEA NKADVNVLTK AKSQ

Human 1-9 PTH is obtained/synthesized comprising an amino acid sequence:
SVSEIQLMH

Human 39-84 PTH is obtained or synthesized comprising an amino acid sequence:
P LAPRDAGSQR PRKKEDNVLV ESHEKSLGEA NKADVNVLTK AKSQ

Human 1-34 PTH is obtained or synthesized comprising an amino acid sequence:
SVSEIQLMHN LGKHLNSMER VEWLRKKLQD VHNF

A 7-34 PTH protein fragment is isolated or synthesized comprising:
LMHN LGKHLNSMER VEWLRKKLQD VHNF

In one embodiment, the 1-9 PTH fragment is attached to an affinity purification column. The attachment may be effected through means known in the art, such as a through the use of a linker at the carboxyl terminal end of the PTH fragment.

In one embodiment, a binding component comprises an antibody specific for the region 1-9 PTH and is prepared as follows. One or more goats are immunized with 1-84 PTH. Thereafter, serum from the immunized goat(s) is/are pooled and affinity purified against 1-9 PTH. After affinity purification against 1-9 PTH, the reaction product of that affinity purification is optionally negatively absorbed with 7-84 PTH. Frequently, this binding component comprises the binding component aspect of the blocking, isolation and/or tracer binding component. Other methods of preparing and purifying antibodies exist and are know in the art, see, e.g., U.S. Pat. No. 6,689,566.

In one embodiment, a binding component comprises an antibody specific for the region 1-34 PTH and is prepared as follows. The 1-34 PTH (1-34) fragment is attached to an affinity purification column. The attachment may be effected through means known in the art, such as a through the use of a linker at the carboxyl terminal end of the PTH fragment. One or more goats are immunized with 1-84 PTH. Thereafter, serum from the immunized goat(s) is/are pooled and affinity purified against 1-34 PTH. After affinity purification against 1-34 PTH, the reaction product of that affinity purification is optionally negatively absorbed with 1-9 PTH. Frequently, this binding component comprises the binding component aspect of the tracer binding component.

In one embodiment, a binding component comprises an antibody specific for the region 7-34 PTH and is prepared as follows. The 7-34 PTH (7-34) fragment is attached to an affinity purification column. The attachment may be effected through means known in the art, such as a through the use of a linker at the carboxyl terminal end of the PTH fragment. One or more goats are immunized with 1-84 PTH. Thereafter, serum from the immunized goat(s) is/are pooled and affinity purified against 7-34 PTH. After affinity purification against 7-34 PTH, the reaction product of that affinity purification is optionally negatively absorbed with 1-9 PTH. Frequently, this binding component comprises the binding component aspect of the tracer binding component.

Although not intending to be bound by any particular theory, the capacity of anti-peptide antibodies to recognize the native protein when utilized in immunoprecipitation or immunohistochemistry staining, depends on the peptide sequence displayed on the surface of the native protein in a conformation similar to that found in the peptide-carrier protein conjugate. Therefore, the successful production of anti-peptide antibodies is often determined by the prediction of the location of certain peptide sequences in the three-dimensional structure of the protein. Protein prediction programs are available for such analysis. Important factors to consider include, for example, protein hydrophilicity, hydropathicity, percent accessible residues, Beta-turn, and flexibility. See Kyte J., Doolittle R. F., 1982. J. Mol. Biol. 157:105-132; Hopp T. P., Woods K. R., 1981. Proc. Natl. Acad. Sci. U.S.A. 78:3824-3828; Janin J., 1979 Nature 277:491-492; Deleage, G., Roux B. 1987 Protein Engineering 1:289-294; and Bhaskaran R., and Ponnuswamy P. K., 1988. Int. J. Pept. Protein Res. 32:242-255. See also the ProtScale website located on the World Wide Web at (.expasy.ch/cgi-bin/protscale.pl) through the ExPasy molecular biology server.

Once produced or obtained, such antigens are useful for generating antibodies thereto using methods known in the art. Frequently, this process involves administering target proteins or peptides to a host animal. Suitable animals include rabbits, mice, sheep, chickens, goats, cows, pigs, rats, etc. A number of other animals may also be suitable for such antibody generation which are readily known and available in the art.

In one embodiment, the negative screening protein or peptide is attached to a solid phase. Frequently, the solid phase is selected from the group consisting of an agarose bead, a cellulose particle, a glass fiber, a controlled pore glass bead and a polystyrene plastic bead. In another frequent embodiment, the solid phase can be separated from the antibody mixture to remove undesired antibodies that bind to the negative screening protein or peptide.

In another embodiment, the antibody mixture is passed through a column comprising the negative screening protein or peptide affixed to a solid phase to retain an undesired antibody that binds to the negative screening protein or peptide in the column while allowing a desired antibody that does not bind to the negative screening protein or peptide to pass through.

In an occasional embodiment, the present methods further comprise a positive screen to collect the desired antibodies. Such positive screening step may be undertaken before, but is frequently undertaken after binding assessment.

In a frequent embodiment, the binding between the target protein or peptide with a specific antibody is assessed by a sandwich or competitive assay format. Frequently, the binding between the target protein or peptide with a specific antibody is assessed by a format selected from the group consisting of an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, u-capture assay, inhibition assay and avidity assay.

To create an affinity-purified anti-(1-9) PTH antibody, one first uses a selected initial PTH sequence peptide (e.g., 1-3 PTH, 1-4 PTH, 1-5 PTH, 1-6 PTH, 1-7 PTH, 1-8 PTH, 1-9 PTH, 1-10 PTH, . . . 1-34 PTH, etc.) as described above as part of an immunogen for injection into a goat. The peptide can be used either by itself as an injectable immunogen, incorporated into a non-PTH peptide having a molecular weight, typically, of between about 5000 and 10,000,000, or as part of the whole 1-84 PTH sequence. The immunogen is mixed with an equal volume of Freunds complete adjuvant which is a mixture of light mineral oil and inactivated mycobacterium tuberculosis bacilli. The resulting mixture is homogenized to produce an aqueous/oil emulsion which is injected into the animal (typically a goat) for the primary immunization. The immunogen dose is approximately 50-400 micrograms. The goats are injected monthly with the same dose of immunogen complex except no mycobacterium tuberculosis bacilli is used in these subsequent injections (Freunds incomplete adjuvant). The goats are bled monthly, approximately three months after the primary immunization. The serum (or antiserum) is derived from each bleeding by separating the red blood cells from the blood by centrifugation and removing the antiserum which is rich in (1-9) PTH antibodies.

To purify the antiserum for one exemplary (1-9) PTH antibody, one packs a separation column with the initial PTH sequence peptide bound beads described above, washes the column and equilibrates it with 0.01 M phosphate buffered saline (PBS). The antiserum is loaded onto the column and washed with 0.01 M PBS in order to remove antibodies without the (1-9) PTH specificity. The bound specific goat anti-(l-9) PTH polyclonal antibody is eluted from the solid phase 1-9 PTH in the column by passing an elution solution of 0.1 M glycine hydrochloride buffer, pH 2.5 through the column. The eluted polyclonal antibody is neutralized after it leaves the column with either the addition of I M phosphate buffer, pH 7.5 or by a buffer exchange with 0.01 M PBS, as is known to those of skill in the art. The polyclonal antibody can be stored at 2-8 degrees centigrade. This process can be utilized to create a variety of PTH antibodies useful as binding components to bind undesired interfering moieties (such as 1-84 PTH for a direct 7-84 PTH assay), capture antibodies, blocking antibodies, and others contemplated herein depending on the PTH fragment that is affixed to the gel in the column. The specific peptide sequences utilized as the immunogen and in the separation column will vary depending on the desired specificity. The anti-1-9 PTH antibody produced by this process avoids cross reactivity with PTH fragments lacking the first amino acid residue of PTH, which could comprise the analytes of interest when the 1-84 PTH could comprise the interfering moiety. Additional antibodies of other specificities are clearly contemplated.

Exemplary capture antibodies can be created by attaching antibodies (e.g., affinity purified goat anti-39-84 PTH antibody (Scantibodies Laboratory, Inc., Santee, Calif.)) to 12×75 mm polystyrene tubes (Nunc, Denmark) by means of passive absorption techniques, or to particles, beads or agarose gel (among other substrates) by methods via covalent or non-covalent bonds, the chemistries of which are known to those of skill in the art.

One can create a labeled antibody by iodinating 50 micrograms of an antibody by oxidation with chloramine T, incubating for 25 seconds at room temperature with 1 millicurie of 125-I radioisotope and reducing with sodium metabisulfate. Unincorporated 125-I radioisotope can be separated from the antibody by passing the iodination mixture over a PD-10 desalting column (Pharmacia, Uppsala, Sweden) and following the manufacturer's instructions. The fractions collected from the desalting column are measured in a gamma counter and those fractions representing the 125-I labeled antibody are pooled and diluted to approximately 300,000 DPM (disintegrations per minute) per 100 microliters. Alternatively, one can obtain labeled or unlabeled antibodies, such as affinity purified goat anti-1-34 PTH antibody or affinity purified goat anti-7-34 PTH antibody, among others, from commercially available sources such as Scantibodies Laboratory, Inc., Santee, Calif.

g. Kits

In a frequent embodiment, a kit is provided in line with methods and materials described herein including specificity by blocking, selective epitope exposure, bead, CAP-PR, PTH drug monitoring, calcitonin measurement, whole PTH measurement and trio kit technologies. These methods and their materials are described elsewhere herein.

The present description provides for kits for carrying out the methods of the invention. Such kits comprise in one or more containers a blocking binding component, a labeled tracer binding component, an assay solid phase binding component (such as an agarose or bead, or a solid surface such as the surface of a tube wall) and optionally an isolation binding component. The reagents will vary based on the intended assay and may further include wash reagents, diluents and other reagents and containers necessary to perform an assay in accordance with the present methods. Often the kit is provided one or more reaction chambers/vessels and reagents for use therein.

In a frequent embodiment, a reagent is provided useful for the pretreatment of a sample to determine the level of 7-84 PTH using a total PTH assay comprising a binding component specific for all or a part of a region on the PTH molecule comprising 1-9 PTH or 1-15 PTH, wherein the binding component is attached to a solid phase. In another frequent embodiment, a reagent useful for the pretreatment of a sample is provided to determine the level of 1-84 PTH using a total PTH assay comprising a binding component specific for all or a part of a region on the PTH molecule comprising 15-34 PTH or 7-34 PTH. In general, these reagents are useful to transform a total or “intact” PTH assay into an assay capable of directly measuring specifically 7-84 PTH or 1-84 PTH, respectively. On occasion, these reagents will be utilized together to specifically analyze the presence and/or level of 1-84 PTH and the presence and/or level of 7-84 PTH in a sample, from which a total PTH level is determined. These reagents are utilized in accordance with the methods provided herein such that the sample is pretreated prior to contacting the sample with the reagents used for the total or “intact” PTH assay that, apart from these sample pretreatment methods, is able to measure both 1-84 PTH and 7-84 PTH.

Instructions are frequently included for use of the contemplated kits and reagents.

h. PTH Assays

Assays useful for the measurement of whole PTH include Scantibodies CAP PTH assay (available from Scantibodies Laboratories, Inc., Santee, Calif.), Scantibodies Whole PTH assay (available from Scantibodies Laboratories, Inc.), Advantage Bio-Intact PTH assay (available from Nichols Institute Diagnostics, San Clemente, Calif.) or Human Bioactive Intact PTH assay (available from Immutopics, Inc., San Clemente, Calif.).

Assays useful for the measurement of total PTH include Scantibodies total intact PTH assay or intact PTH assay (available from Scantibodies Laboratories, Inc.), Allegro Intact PTH Assay (available from Nichols Institute Diagnostics), Advantage Intact PTH Assay (available from Nichols Institute Diagnostics), or Human Intact PTH assay (available from Immutopics). See, e.g., Slatopolsky E, et al., Kidney Intl. 2000; 58:753-761 (demonstrating that both that the Nichols Allegro intact PTH IRMA test measures both 1-84 PTH and 7-84 PTH); see also Lepage, R., et al., Clin. Chem. (1998) 44(4):805-9. Other total PTH assays are available or known in the art and are contemplated herein such as those available from Diagnostics Products Corp. (Los Angeles, Calif.) (e.g., the Immulite intact PTH test or the Immulite Turbo intact PTH assay), among others.

In one embodiment, a kit component is contemplated that is useful together with known PTH assays. For example, by adding a 1-9 PTH blocking binding component in the form of a particle or a blocking antibody pretreatment to the sample, any of the above referenced total PTH assays can be converted into a specific and direct 7-84 PTH assay. Other assay reagents contemplated herein can be utilized in a similar fashion consistent with their purpose in the overall assay.

In one embodiment, it is further understood that if a total PTH assay measures 7-84 PTH together with other PTH fragments, and if that total PTH assay is made by the above stated methods to not measure 1-84 PTH (e.g., via the use of a 1-9 PTH blocking binding component), the resulting “direct and specific 7-84 PTH assay” will frequently measure 7-84 PTH together with the other PTH fragments that the total PTH assay measured before being subjected to any of the present methods.

Frequently, binding between the analyte and/or the interfering moiety and the binding component is assessed by a format selected from the group consisting of, e.g., an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, chemiluminescence assay, lateral flow immunoassay, immuno radio metric assay (IRMA), μ-capture assay, linear flow membrane chromatography, inhibition assay, energy transfer assay, avidity assay, turbidometric immunoassay and time resolved amplified cryptate emission (TRACE) assay.

i. Exemplary Analytes

In one embodiment, the analyte of interest is a marker of a biological pathway, a group of cellular structures with identical or similar biological function, a stage of cell cycle, a cell type, a tissue type, an organ type, a developmental stage, a disease or disorder type or stage, or a drug or other treatment. Frequently, the analyte of interest is a clinical marker. Often, the analyte of interest is a marker of parathyroid gland disease status, renal bone disease, osteoporosis, bone turnover status, Graves disease, the extent of partial or complete parathyroid gland removal. On occasion, the analyte of interest is a hormone or a non-proteineous or non-peptidyl moiety such as an oligonucleotide, a nucleic acid, a vitamin, an oligosaccharide, a carbohydrate, a lipid, a small molecule and a complex or combination thereof. Similarly, interfering moieties can be any of these types of moieties.

The analytes contemplated in the present disclosure generally contain some homology to the interfering moiety. Often this homology is significant. Thus, often the analyte comprises a fragment, isoform, analog, variant or homologous mutant of the interfering moiety. An analyte comprising an isoform or analog refers to a moiety that is related to and/or derived from the interfering moiety.

Often, the analyte is greater than about 90% homologous or identical to the interfering moiety, although higher percentages are clearly contemplated and preferred. For example, analytes of the present disclosure may be about 91%, about 92%, about 93%, about 94%, about 95%, about 96%, about 97%, about 98%, about 99%, and about 99% or more homologous or identical to the corresponding interfering moiety. Less occasionally, the analyte of the present disclosure is greater than about 15%, or about 50%, or about 60%, or about 70%, or greater than about 75%, or greater than about 80%, or greater than about 85% homologous or identical to the corresponding interfering moiety.

In a frequent embodiment, the present methods are used for prognosis, diagnosis and/or treatment monitoring of familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, Graves disease, the extent of parathyroid gland surgical removal, oversuppression with vitamin D or a vitamin D analogue or a calcimimetic or calcium and chronic uremia. Often, the hyperparathyroidism is primary hyperparathyroidism caused by primary hyperplasia or adenoma of the parathyroid glands or secondary hyperparathyroidism caused by renal failure.

In an occasional embodiment, the methods are used to distinguish among gastric inhibitory polypeptide (GIP) and/or glucagon-like peptide (GLP), GIP-1 and/or GLP-1, and GIP-2 and/or GLP-2. Also on occasion, the methods are used to distinguish among creatine kinase (CK) isoforms (CK-MM (muscle) and CK-BB (brain) and CK-MB (hybrid)), e.g., used to distinguish CK-MM from CK-BB. Also on occasion, the methods are used to distinguish thyroid stimulating hormone (TSH) and/or follicle stimulating hormone (FSH). In addition, the present methods are useful for distinguishing insulin from proinsulin or measuring either analyte. Further, the present methods are useful for distinguishing osteocalcin or adrenocorticotrophic hormone (ACTH) from their fragments. Frequently, the present methods are utilized to distinguish between insulin, proinsulin. Also frequently, the present kits and methods are used to distinguish between calcitonin or procalcitonin.

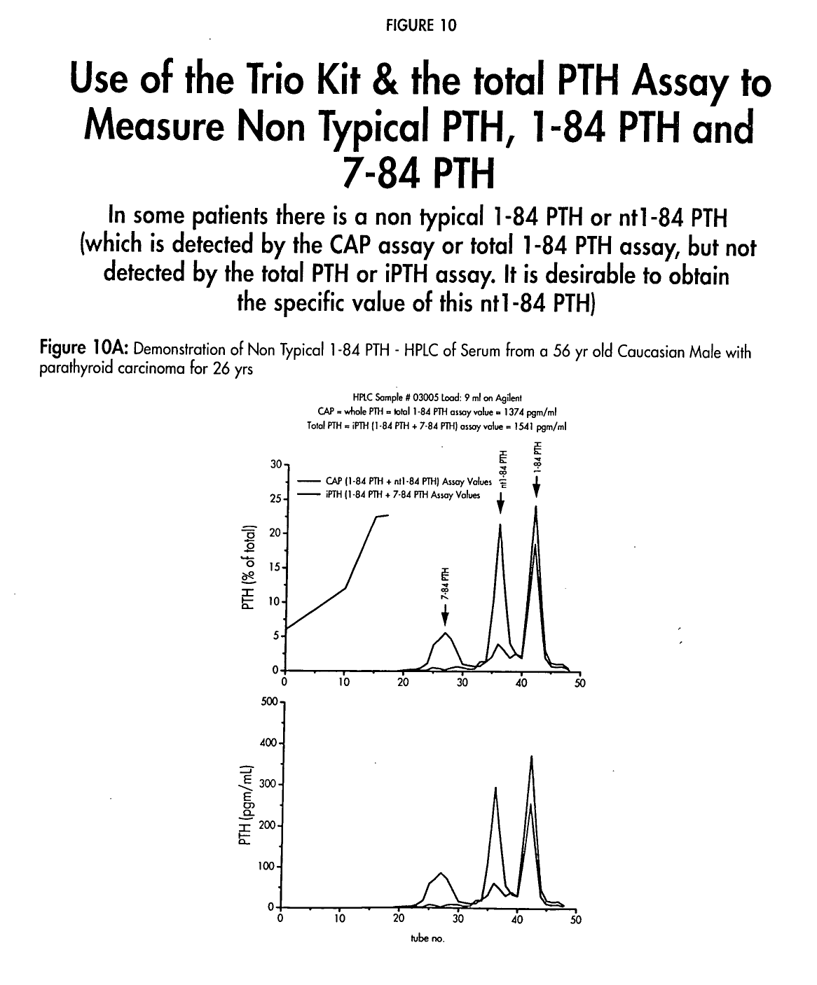

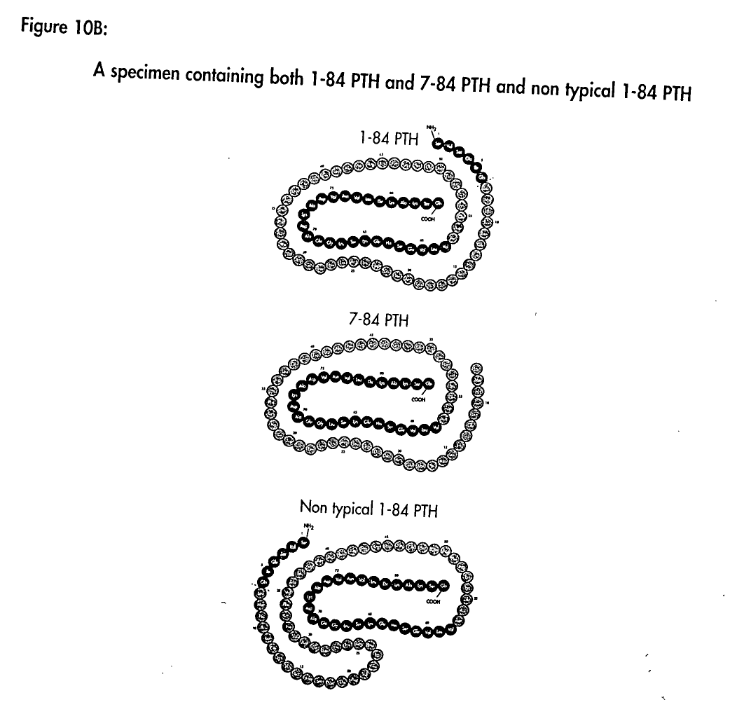

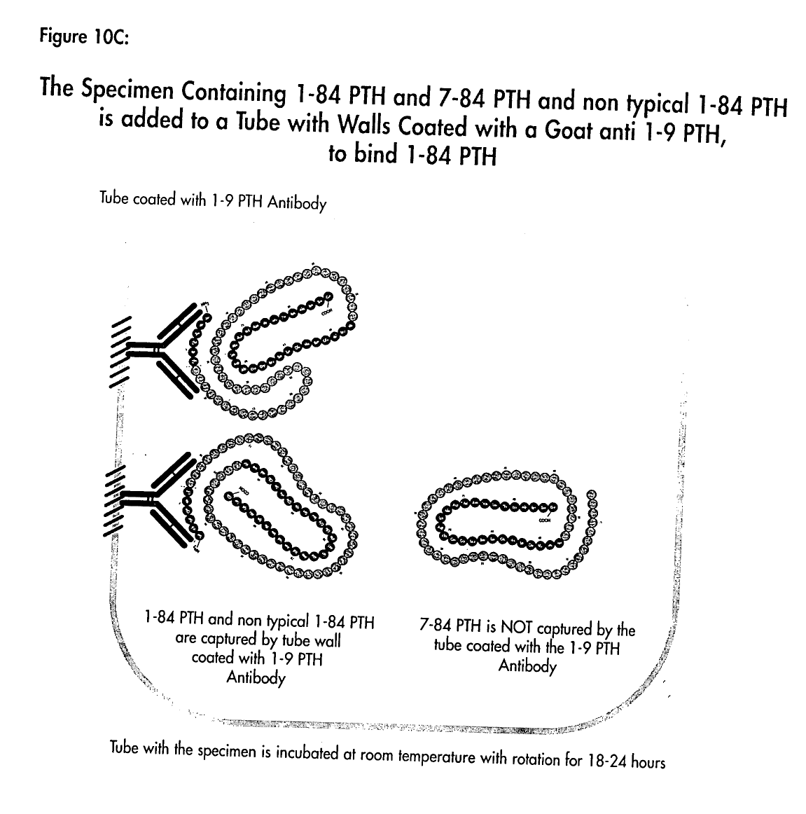

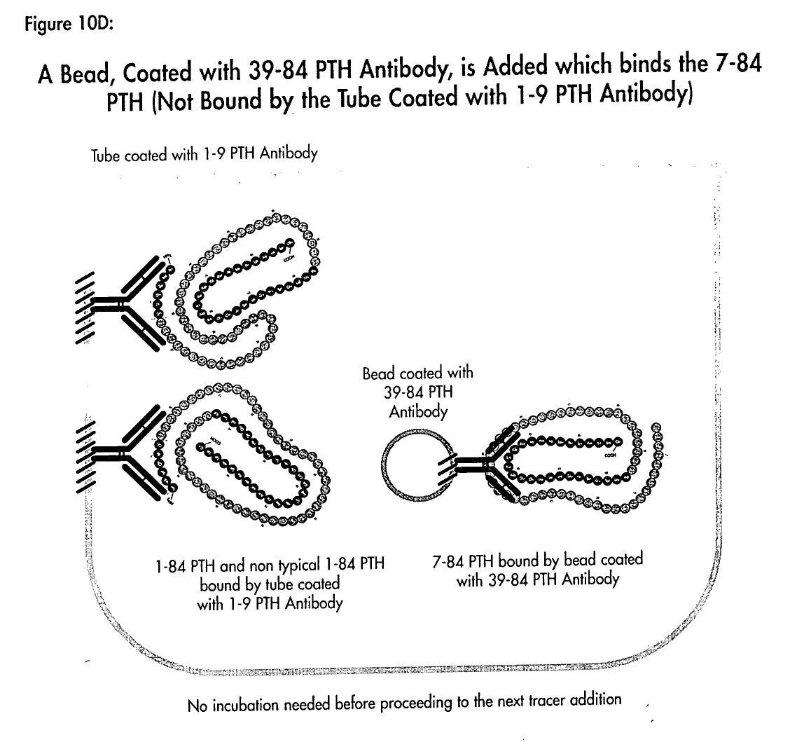

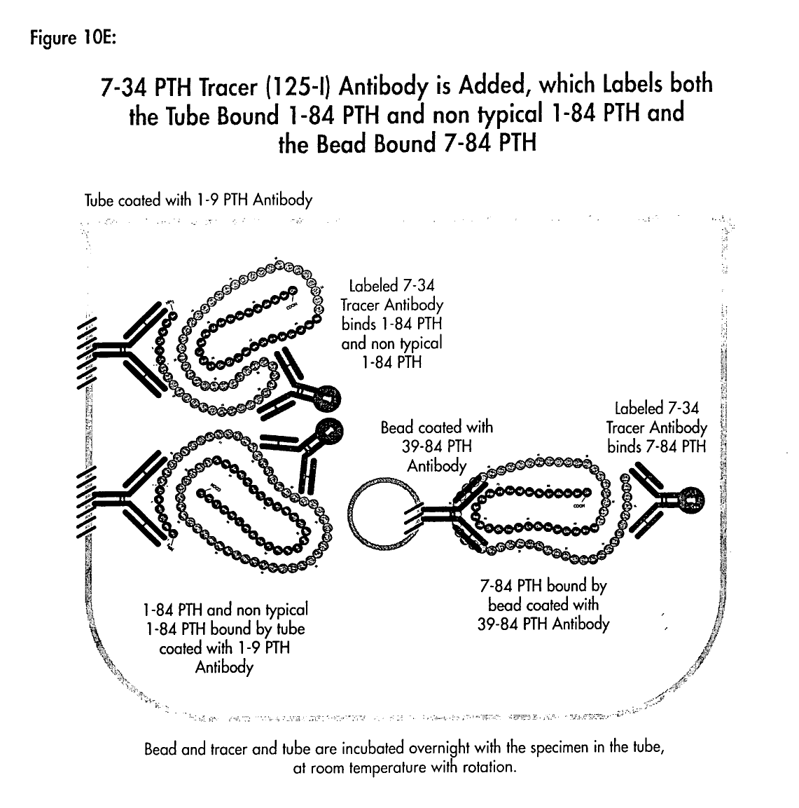

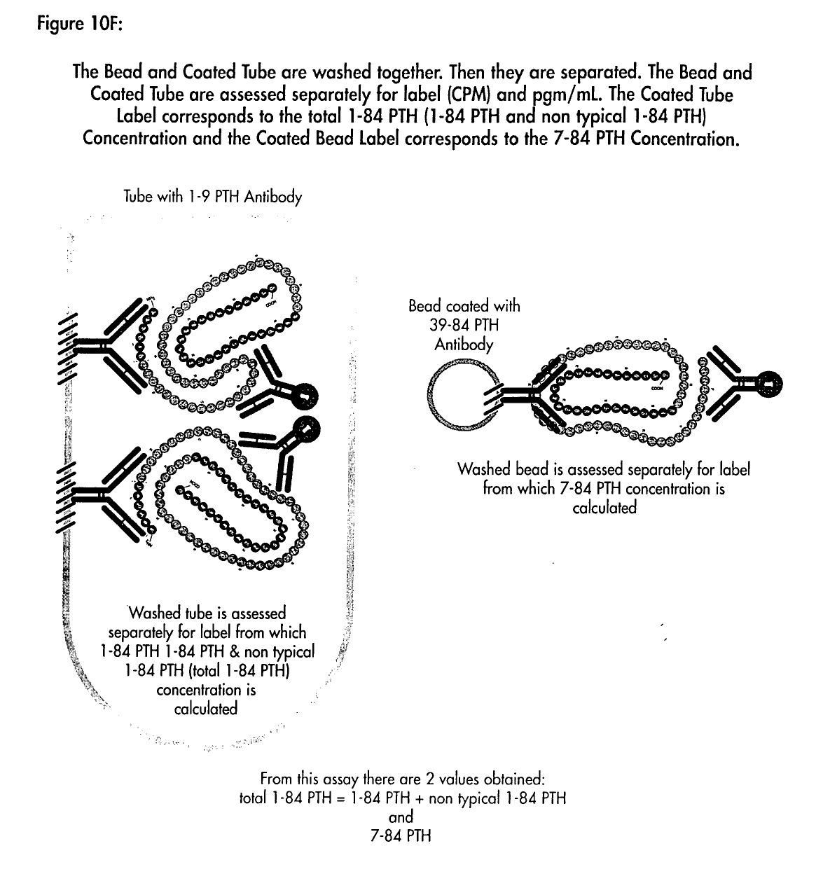

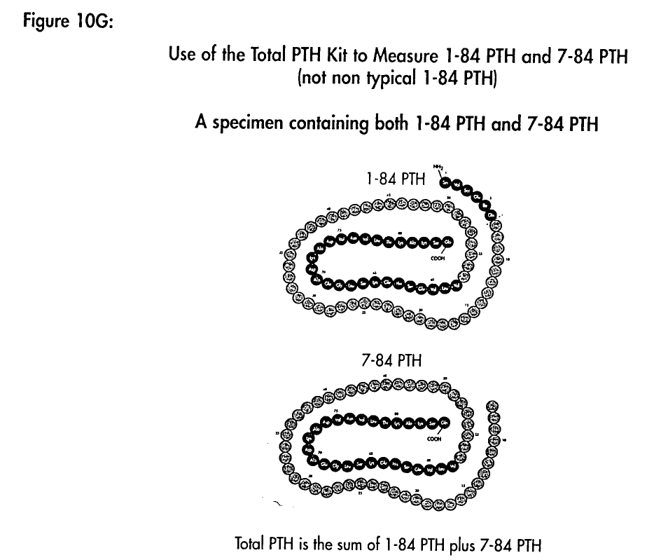

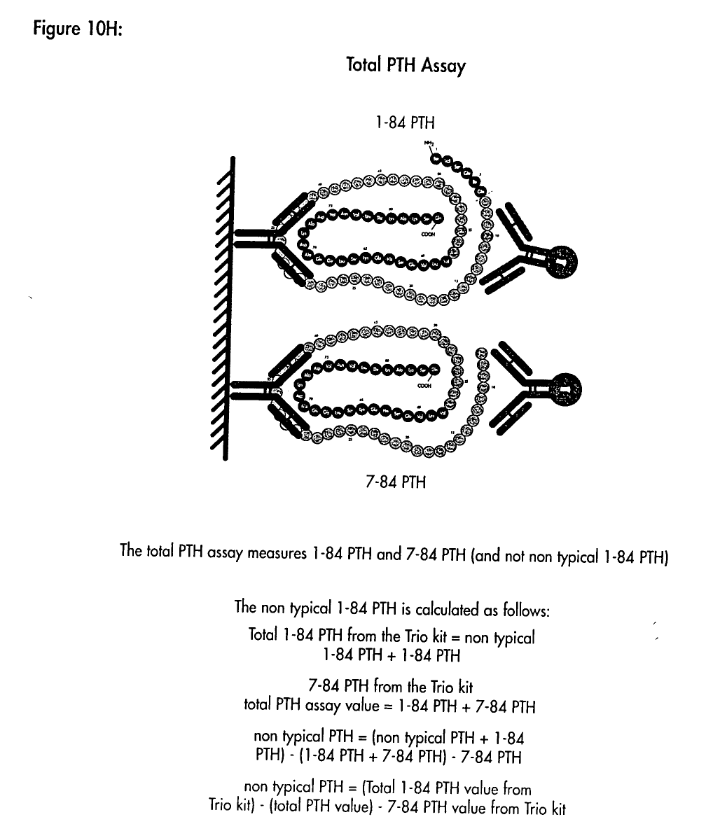

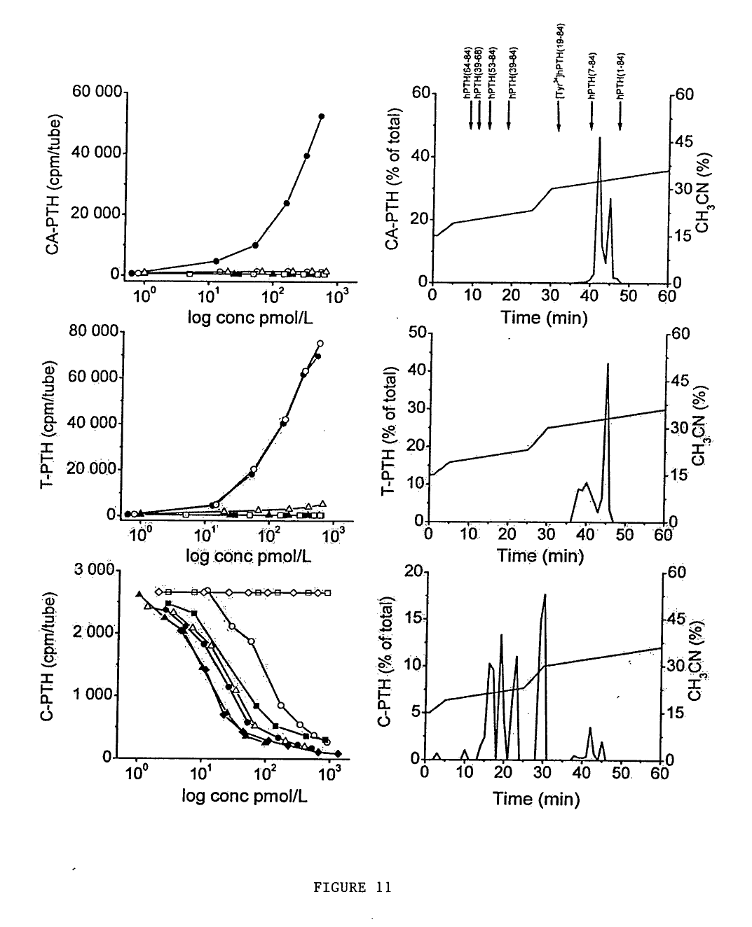

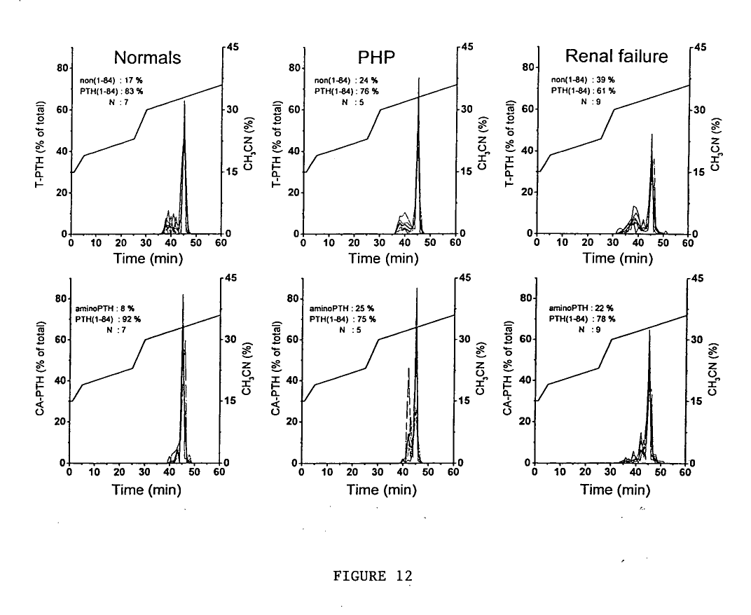

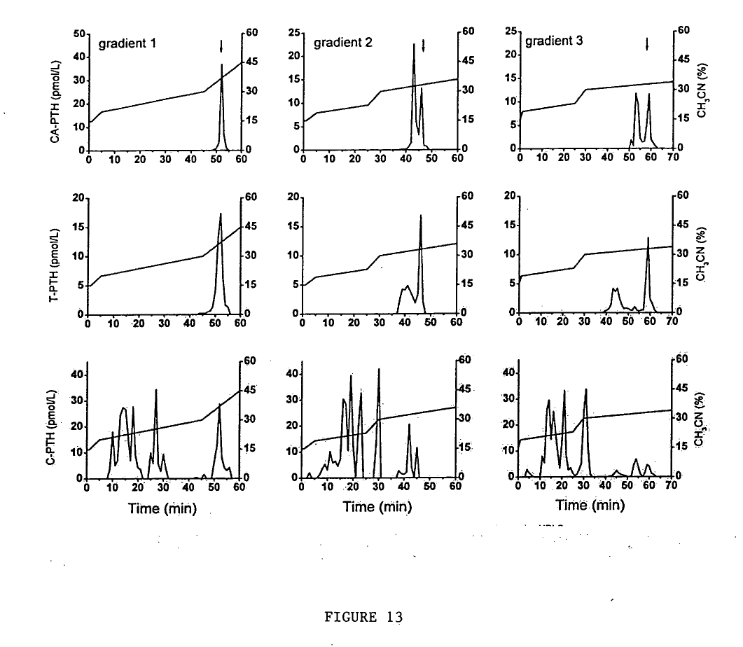

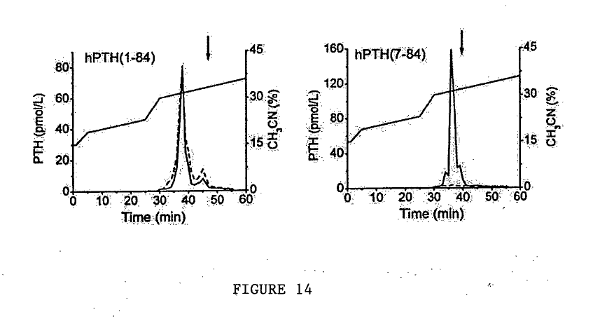

The present methods are also useful to measure and/or compare the presence and/or level of non-typical PTH (ntPTH) (also referred to herein as non-typical 1-84 PTH or a new form of PTH (nfPTH)). See U.S. Application Ser. No. 60,508,547, filed Oct. 3, 2003. As indicated in the HPLC profile of FIG. 10A, three peaks are present. These peaks represent a percentage of the total PTH in a sample. The sample was obtained from a subject afflicted with parathyroid cancer. Peak 1 corresponds to a non-whole PTH fragment comprising 7-84 PTH. Peak 2 corresponds to ntPTH. Peak 3 corresponds to whole PTH. As the profile indicates, ntPTH is chromatographically different from 1-84 PTH and it is measured by a specific 1-84 PTH assay (e.g., the CAP or Whole PTH assay—which measures peaks 2 and 3), but it is not measured to any significant extent by a total PTH assay (e.g., the total intact PTH or intact PTH assay—which measures peaks 1 and 3). These two assays, together with an optional direct measurement of 7-84 PTH, enable the measurement of ntPTH by subtracting the 7-84 level from the measured total PTH assay value, which is then subtracted from the measured 1-84 PTH assay value. The 7-84 level in this comparison can be determined utilizing any of the methods set forth herein.

As further depicted in FIG. 10B-H, an exemplary method for the determination of ntPTH is the following. First, a sample is obtained containing, or suspected of containing, both 1-84 PTH and 7-84 PTH and ntPTH (10B). The sample is then added to a tube having walls coated with a goat anti 1-9 PTH. 1-84 PTH and non-typical 1-84 PTH, if present, are captured/bound by the anti-1-9 PTH antibody on the tube wall. 7-84 PTH, if present, is not captured by the tube coated with the anti-1-9 PTH antibody (10C). This tube with the sample is incubated at room temperature with rotation for 18-24 hours. A bead coated with anti-39-84 PTH antibody, is added to the tube, which antibody binds the 7-84 PTH present in the sample (10D). No incubation is needed before proceeding to the next step. Anti-7-34 PTH tracer (125-I) antibody is added, which binds and labels both the 1-84 PTH and non-typical 1-84 PTH bound to the tube wall and the 7-84 PTH bound to the bead (10E). The bead and tracer and tube are incubated overnight with the specimen in the tube, at room temperature with rotation. The bead and coated tube are washed together, then separated. The bead and coated tube are assessed separately for label (CPM) and pgm/mL (10F). The label detected on the coated tube corresponds to the total 1-84 PTH (comprising 1-84 PTH and non-typical 1-84 PTH) concentration, and the label detected on the coated bead corresponds to the 7-84 PTH concentration. Another related and/or parallel sample containing both 1-84 PTH and 7-84 PTH is measured utilizing a Total PTH Kit to measure 1-84 PTH and 7-84 PTH (but not the non-typical 1-84 PTH). This total PTH comprises the sum of 1-84 PTH plus 7-84 PTH (10G). The non-typical 1-84 PTH is then calculated by determining the 1-84 PTH level value and subtracting that value from the combined non-typical PTH and 1-84 PTH level value (10H). Exemplary alternative equations (based on the present methods) for calculating a non-typical PTH value in a sample are the following:
a. Non-typical PTH=(non-typical PTH+1-84 PTH)−(1-84 PTH+7-84 PTH)−7-84 PTH
b. Non-typical PTH=(Total 1-84 PTH value from Trio kit)−(total PTH value)−7-84 PTH value from Trio kit

The level of ntPTH can then be utilized to diagnose, monitor and/or guide treatment for a disease or disorder. Frequently, the disease or disorder comprises a renal bone disease, including adynamic bone disease, high bone turnover disease and osteoporosis. Also frequently, the ntPTH level is compared with the 1-84 PTH, 7-84 PTH and/or total PTH levels in a ratio to diagnose, monitor and/or guide treatment for a disease or disorder. The gate, threshold and/or algorithm methods described herein may be utilized in the diagnosis, monitoring or guiding of treatment for a disease or disorder.

Also frequently, a method is provided for monitoring, diagnosing and/or guiding treatment for a disease or disorder comprising evaluating the level of a specific analyte or interfering moiety in a sample, wherein if the measured analyte or interfering moiety level is at a specific, often pre-designated, level the subject is at risk for or has a specific disease or disorder. Often the analyte (and the level thereof), the interfering moiety (and the level thereof), and/or the specific disease or disorder is/are pre-designated. Further, based on the measured level of the analyte or interfering moiety, it is often determined that a ratio of the one or more analytes and/or interfering moieties should be utilized to monitor, diagnose and/or guide treatment for a disease or disorder. In this embodiment, the level of the analyte and/or interfering moiety is utilized as a gate indicating when the use of a ratio based evaluation of the sample would be appropriate or medically indicated. For example, often the level of an analyte or interfering moiety may be present at a specific level (often a high or low level) that it provides an indication of a specific disease or disorder, without resorting to a ratio analysis.

Other features and advantages of the invention will be apparent from the following description.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

### EXAMPLES

### Example 1

Control preparations are prepared as follows:
1. 1000 frozen control tubes with 500 pgm/ml of 1-84 PTH
2. 1000 frozen control tubes with 500 pgm/ml of 7-84 PTH
3. 1000 frozen control tubes with 500 pgm/ml of 1-84 PTH and 500 pgm/ml of 7-84 PTH.

Affinity purified antibody preparations are prepared as follows:

Prepare 2 mg of the following affinity purified goat anti-PTH antibodies with the following specificities:
1. 1-9 PTH antibody
2. 1-13 PTH antibody
3. 1-15 PTH antibody
4. 1-18 PTH antibody
5. 1-20 PTH antibody
6. 1-22 PTH antibody
7. 1-25 PTH antibody
8. 1-27 PTH antibody
9. 1-30 PTH antibody
10. 25-34 PTH antibody

Tracer and capture binding components are prepared as follows:

Prepare 10 tracer and capture antibodies, each comprising a rough preparation that can demonstrate using existing standard curves a signal difference between 100, 500, 1000 and 2000 pgm/ml:
1. 1-9 PTH antibody tracer with 39-84 PTH antibody capture (obtained from Scantibodies, Inc., Santee, Calif.)
2. 1-13 PTH antibody tracer with 39-84 PTH capture
3. 1-15 PTH antibody tracer with 39-84 PTH capture
4. 1-18 PTH antibody tracer with 39-84 PTH capture
5. 1-20 PTH antibody tracer with 39-84 PTH capture
6. 1-22 PTH antibody tracer with 39-84 PTH capture
7. 1-25 PTH antibody tracer with 39-84 PTH capture
8. 1-27 PTH antibody tracer with 39-84 PTH capture
9. 1-30 PTH antibody tracer with 39-84 PTH capture
10. 1-34 PTH antibody tracer with 39-84 PTH capture (obtained from Scantibodies, Inc., Santee, Calif.)

Beads are prepared as follows:

Prepare 200 beads with the following antibodies (the beads are avidin coated and have attached to them the following biotinylated antibodies):
1. 1-9 PTH antibody
2. 1-13 PTH antibody
3. 1-15 PTH antibody
4. 1-18 PTH antibody
5. 1-20 PTH antibody

Control specimens are prepared for assay:

The control samples, with the exception of the bead treated control samples, are incubated at room temperature for 18-24 hours with rotation at 170 rpm in the following manner (all additions of antibodies do not dilute the controls by more than 10%):

Control
3 Controls

Specificity by Blocking
1. 3 Controls plus 2 μgms of 1-9 PTH antibody
2. 3 Controls plus 2 μgms of 1-13 PTH antibody
3. 3 Controls plus 2 μgms of 1-15 PTH antibody
4. 3 Controls plus 2 μgms of 1-18 PTH antibody
5. 3 Controls plus 2 μgms of 1-20 PTH antibody

Selective Epitope Exposure
1. 3 Controls plus 2 μgms of 1-9 PTH antibody & 2 μgms of 25-34 PTH antibody
2. 3 Controls plus 2 μgms of 1-13 PTH antibody & 2 μgms of 25-34 PTH antibody
3. 3 Controls plus 2 μgms 1-15 PTH antibody & 2 μgms of 25-34 PTH antibody
4. 3 Controls plus 2 μgms of 1-18 PTH antibody & 2 μgms of 25-34 PTH antibody
5. 3 Controls plus 2 μgms of 1-20 PTH antibody & 2 μgms of 25-34 PTH antibody

Bead Technology
1. 400 microliters of each of 3 Controls plus 1-9 PTH Antibody bead that is removed after 18-24 hour incubation at room temperature with 170 rpm rotation—remove 200 microliters of the pretreated control and assay in a total PTH assay (obtained from Scantibodies Laboratories, Inc.).
2. 400 microliters of each of 3 Controls plus 1-13 PTH Antibody bead that is removed after 18-24 hour incubation at room temperature with 170 rpm rotation—remove 200 microliters of the pretreated control and assay in a total PTH assay
3. 400 microliters of each of 3 Controls plus 1-15 PTH Antibody bead that is removed after 18-24 hour incubation at room temperature with 170 rpm rotation—remove 200 microliters of the pretreated control and assay in a total PTH assay
4. 400 microliters of each of 3 Controls plus 1-18 PTH Antibody bead that is removed after 18-24 hour incubation at room temperature with 170 rpm rotation—remove 200 microliters of the pretreated control and assay in a total PTH assay
5. 400 microliters of each of 3 Controls plus 1-20 PTH Antibody bead that is removed 18-24 hour incubation at room temperature with 170 rpm rotation—remove 200 microliters of the pretreated control and assay in a total PTH assay.

Assay data are then evaluated as follows:

The assay value for the control tube with the 1-84 PTH plus the 7-84 PTH is used to compare with the assay value from the other 3 control tubes for a given assay (alternatively one can use CPM):
1. (7-84 PTH control assay value with no pretreatment)−(1-84 PTH control assay value with pretreatment)×100%/(7-84 PTH control assay value with no pretreatment)
2. (7-84 PTH control assay value with no pretreatment)−(7-84 PTH control assay value with pretreatment)×100%/(7-84 PTH control assay value with no pretreatment)
3. (7-84 PTH control assay value with no pretreatment)−(1-84 PTH & 7-84 PTH control assay value with pretreatment)×100%/(7-84 PTH control assay value with no pretreatment)

Subjects are evaluated in accordance with the present methods, for example, each of the above pretreatment methods are utilized to treat samples, which are assayed utilizing one or more PTH assays.

In a further example, new assays for the specific measurement of 7-84 PTH (and/or another PTH fragment), for use optionally together in the 1-84 PTH/7-84 PTH ratio are validated by bone biopsy studies.

### Example 2

5 mg of affinity purified goat anti 1-9 PTH is coupled to beads obtained from Pierce Biotechnology (Rockford, Ill.), AMINOLINK® Kit. The beads are provided in a 4% cross-linked beaded agarose support that is activated to form aldehyde functional groups that develop a stable bond. The beads range in size from 4-100 microns. The coupled beads are diluted such that 2 micrograms are in 10 microliters and this 10 microliters is added to 200 microliters of patient plasma. The beads and patient sample are incubated with rotation for 5 hours at room temperature. As a result of the incubation, 1-84 PTH present in the sample binds the goat anti 1-9 PTH coupled to the beads. Agarose beads such as SEPHAROSE® 4B (Pharmacia Fine Chemicals, Inc., Piscataway, N.J.) beads that are cyanogen bromide activated may also be utilized. Cyanogen bromide activation is useful to couple, through covalent bonding, proteins such as particular binding components to the beads.

If the beads are to be removed from the sample prior to assay, the 200 microliters of patient plasma and the 10 microliters of coupled beads are added into a centrifuge column (obtained from Pierce Biotechnology, Inc., Rockford, Ill.) and the mixture is rotated for 18-24 hours at 170 rpm at room temperature in the column. Following the incubation, the bottom of the column is broken off and it is placed in a microcentrifuge tube and spun to remove the patient plasma without the 1-84 PTH. The patient sample aspirated/removed from the microcentrifuge tube is then assayed for the 7-84 PTH level.

Alternatively, if the beads are to remain in the sample, the 200 microliters of patient plasma and the 10 microliters of coupled beads are added to an assay tube and the mixture is rotated at 170 rpm and incubated for 18-24 hours at room temperature. Following the incubation a labeled tracer antibody that will specifically bind 7-84 PTH and an assay bead comprising an antibody affinity purified against 39-84 PTH are added, and the total PTH assay proceeds without the removal of the coupled bead. 1-84 PTH coupled to the 1-9 PTH antibody bead will not participate in the assay reaction (as it is out of solution) and will, therefore, not be detected. In contrast, 7-84 PTH bound by a detectible tracer antibody, will be detected in the assay.

### Example 3

A parallel patient sample to that obtained in Example 2 is obtained and assayed by a traditional assay that measures total PTH levels. The total PTH assay results for the sample are then subtracted from the results of the direct 7-84 PTH assay results of Example 2 to determine the 1-84 PTH level in the sample.

### Example 4

1000 polystyrene 12×75 mm tubes (Nunc, Denmark) are coated by conventional tube coating methods known to those of skill in the art such that 2 micrograms of affinity purified goat anti-1-9 PTH antibody are used for coating each tube.

200 microliters of sample is added to a 1-9 PTH antibody coated tube for each sample. A single tube is used for each of the following 200 microliter samples:
1. the controls listed in Example 1 above;
2. a set of standard calibrators containing from 5-2000 pgm/ml of 1-84 PTH;
3. a set of standard calibrators containing from 5-2000 pgm/ml of 7-84 PTH;
4. a set of standard calibrators containing from 2.5-1000 pgm/ml of 1-84 PTH in combination with 2.5-1000 pgm/ml of 7-84 PTH (combined 1-84 PTH plus 7-84 PTH calibrators);
5. 25 EDTA plasma specimens from normal subjects; and
6. 25 EDTA plasma specimens from ESRD patients.

The tubes are incubated by rotating them for 18-24 hours at room temperature with 170 rpm rotation. After this incubation a 3/16 inch polystyrene bead that has been previously coated with affinity purified goat anti-39-84 PTH antibody is added to each tube. 100 microliters of approximately 300,000 cpm of 125-I labeled affinity purified goat anti-1-34 PTH antibody is also introduced to each tube. The 39-84 PTH antibody coated beads are further incubated for 18-24 hours at room temperature with 170 rpm rotation. Following the incubation, the 39-84 PTH antibody coated beads are washed in the 1-9 PTH antibody coated tubes, thus washing both the 39-84 PTH antibody coated beads and the 1-9 PTH antibody coated tubes. The combination of the 1-9 PTH antibody coated tubes and the 39-84 PTH antibody coated beads is quantitatively assessed for 125-I in a gamma counter. Through the use of the combined set of calibrators/standards made up of 1-84 PTH and 7-84 PTH, a standard curve is constructed. The specimens from normal subjects and ESRD specimens are counted in the gamma counter and, based on the combined 1-84 PTH and 7-84 PTH standards, the amount of total PTH is calculated.

The 39-84 PTH antibody coated bead is then transferred from each of the 1-9 PTH antibody coated tubes to a 12×75 mm polystyrene tube that is uncoated and unused for assay purposes. Through the use of the 7-84 PTH standards/calibrators, a standard curve is constructed. The specimens from normal subjects and ESRD specimens are counted in the gamma counter and, based on the 7-84 PTH standards, the amount/concentration of 7-84 PTH in the patient and normal samples is calculated.

The 1-9 PTH antibody coated tubes from which the beads were separated are then counted by themselves without the beads. The separate 1-9 PTH antibody coated tubes from the 1-84 PTH standards/calibrators are used to calculate 1-84 PTH concentrations. The individually assessed 1-84 PTH and 7-84 PTH concentrations from the separated 1-9 PTH antibody coated tubes and 39-84 PTH antibody coated beads are then compared with the combined 1-9 PTH antibody coated tubes and 39-84 PTH antibody coated beads to determine the accuracy of the total PTH concentration. In this step, the total obtained from the gamma counted combined 1-9 PTH antibody coated tube and 39-84 PTH antibody coated bead is compared with the total of the sum of the 1-84 PTH concentration from the assessed separate 1-9 PTH antibody coated tube plus the assessed separate 39-84 PTH antibody coated bead.

The standards/calibrators are further useful to identify cross-over analyte detection in the tubes such that, for example, 1-84 PTH is inadvertently detected in the tube containing the beads and/or 7-84 is inadvertently detected in the tube with the coated walls after separation of these analytes. Cross-over analyte detection may result if, for example, 1-84 PTH inadvertently binds the 39-84 coated bead and is then transferred to the uncoated tube for detection. Cross-over analyte detection may also result if, for example, 7-84 PTH inadvertently binds the coated tube and is counted after transfer out of the coated bead.

In a frequent embodiment, the levels of 1-84 PTH and 7-84 PTH in a sample are determined directly in separate tubes and the total PTH is calculated by adding the values/levels determined for 1-84 PTH and 7-84 PTH according to the above methods.

### Example 5

This study is also described in D'Amour et al., Clin. Chem., 49(12):2037-2044 (2003), the content of which is incorporated by reference in its entirety.

I. Materials and Methods

A. Subjects

Seven normal individuals and 5 patients with primary hyperparathyroidism (PHP) participated in this study.

B. Methods

Blood was obtained from each of the 5 patients with primary hyperparathyroidism (PHP). For RF, 8 pools were constituted at various PTH concentrations (5.8 to 86.8 pmol/L), these pools were formed from serum that was left over following routine renal failure patient PTH determinations. Although it is possible that pooling may have resulted in PTH forms not representative of specimens from individual subjects, we have found that this approach has given results similar to those obtained in single individuals in the past. See Brossard J H, et al., J Clin Endocrinol Metab 1996;81:3923-3929. Parameters of phosphocalcic metabolism and basal PTH levels were measured in serum or pools of serum either fresh or kept at −90° C. The same sera were used for HPLC analysis.

Synthetic PTH peptides, hPTH(1-84), hPTH(7-84), hPTH(39-84), hPTH(53-84), hPTH(39-68) and hPTH(64-84) were purchased from BACHEM (Torrance, Calif., USA). Mutated [Tyr³⁴]hPTH(19-84) was generously provided by H. Juppner of the Massachusetts General Hospital in Boston, USA. Total calcium, phosphate, alkaline phosphatase and creatinine were measured by automated colorimetric methods.

Total (T)-PTH and CA-PTH were quantitated with commercial kits provided by Scantibodies Laboratory, Inc. (Santee, Calif., USA). C-PTH was measured by a radioimmunoassay reacting predominantly with C-PTH fragments (see D'Amour P, et al., Am. J. Physiol. 1986;251 (Endo Metab):E680-E687; D'Amour P, et al., J Clin Endocrinol Metab 1992;75:525-532; Brossard J H, et al., J Clin Endocrinol Metab 1996;81:3923-3929). Assay specificities were studied with the use of various PTH standards and also through determinations of assay capacities to recognize circulating PTH molecular forms following elution from HPLC profiles. PTH forms from all sera were extracted on Sep-Pak Plus C-18 cartridges (Waters Chromatography Division, Milford, Mass.) (Bennett H P, et al., J Biol Chem 1984;259:2949-2955). PTH molecular forms were further separated on C₁₈ μBondapak analytical columns, 3.9×300 mm (Waters), using different acetonitrile gradients (15 to 50%) in 0.1% trifluoroacetic acid, delivered at 1.5 ml/min over various time periods with a Model 2700 Solvent Delivery System (Bio-Rad, Mississauga, Ontario, Canada) (Brossard J H, et al., J Clin Endocrinol Metab 1996;81:3923-3929; Brossard J H, et al., J Clin Endocrinol Metab 1993;77:413-419; Lepage R, et al., Clin Chem 1998;44:805-809; Brossard J H, et al., Clin Chem 2000;46:697-703). The 1.5-ml fractions were evaporated, freeze-dried, reconstituted to 1 ml with 0.7% BSA in water, and adequate volumes were then measured in the various PTH assays. Control experiments were performed with hPTH(1-84) standard added to hypoparathyroidism serum to insure PTH degradation did not occur during the various procedures. A single peak of immunoreactivity coeluting with hPTH(1-84) was always identified by the T-PTH and CA-PTH and C-PTH assays. Immunoreactive PTH recovery through each of these procedures was better than 75%.

C. Data Analysis

The results in the tables below are expressed as mean values±SD. Differences between groups were analyzed by ANOVA, followed by the Student-Newman-Keuls multiple comparisons test. HPLC profiles were evaluated planimetrically with Origin 4.1 software (Microcal Software, Northbramton, Mass., USA).

FIG. 2 illustrates the immunoreactivity of the three PTH assays using PTH standards or circulating PTH molecular forms separated by HPLC in a PHP patient. The CA-PTH assay reacted with the hPTH(1-84) standard and not with the synthetic C-PTH fragments, including hPTH(7-84). With a more efficient acetonitrile gradient, this assay identified 2 HPLC peaks. The first, in positions 42-43, migrated in front of the hPTH(1-84) peak in position 45. The T-PTH assay behaved like previously reported typical I-PTH assays (see Lepage R, et al., Clin. Chem. 44(4):805-9 (1998)) and was demonstrated to react with equimolarily of hPTH(1-84) and hPTH(7-84) and not with any of the smaller C-PTH fragments. This assay identified hPTH(1-84) in position 45 as well as a broader region in positions 36 to 41 corresponding to non-(1-84)PTH. The C-PTH assay reacted only with PTH fragments having an intact (65-84) structure. It did not react with the mid-carboxyl-terminal fragment hPTH(39-68). Its affinity for various PTH molecules varied, being highest for hPTH(39-84), intermediate for hPTH(1-84), and lowest for hPTH(7-84). It reacted with the molecular forms detected by the CA-PTH and T-PTH assays and also with several less hydrophobic C-PTH fragment peaks migrating in positions 15, 16, 19, 23 and 29.

Table 1 (below) summarizes the biochemical characteristics of the three groups studied. Patients with PHP were hypercalcemic with greater than normal levels of alkaline phosphatase, CA-PTH, T-PTH and C-PTH than NI. Pools of serum from PHP patients also had greater than normal levels of creatinine, phosphate, alkaline phosphatase, CA-PTH, T-PTH and C-PTH than NI. Results are presented in the form of mean±SD. Statistical analysis by both ANOVA and the Student-Newman-Keuls test followed. NI vs. PHP or RF: a, p<0.001; b, p<0.01; c, p<0.05. PHP vs. RF: d, p<0.001; e, p<01; f, p<0.05.

TABLE 1
Biochemical characteristics of the various groups

| | Normal | Primary | Renal |
|---|---|---|---|
| Parameters | Individuals | Hyperparathyroidism | Failure |
| Subjects | 7 | 5 | 8 |
| Calcium (total) | 2.36 ± 0.08 | 2.79 ± 0.19^{a} | 2.20 ± 0.09^{c,d} |
| (mmol/L) | | | |
| PO₄ (mmol/L) | 1.16 ± 0.10 | 1.00 ± 0.35 | 1.76 ± 0.14^{a,d} |
| Alk. Phos. | 58.0 ± 15.3 | 93.4 ± 34.2^{c} | 125.9 ± 52.4^{b} |
| (UI/L) | | | |
| Creatinine | 70.6 ± 11.1 | 97.2 ± 40.7 | 835.6 ± 57.0^{a,d} |
| (μmol/L) | | | |
| T-PTH | 3.13 ± 0.37 | 25.7 ± 26.0^{b} | 47.0 ± 35.1^{a} |
| (pmol/L) | | | |
| CA-PTH | 2.29 ± 0.33 | 23.1 ± 24.2^{b} | 33.4 ± 26.1^{a} |
| (pmol/L) | | | |
| C-PTH | 8.03 ± 1.71 | 79.9 ± 95.8^{b} | 157.4 ± 108.1^{a,f} |
| (pmol/L) | | | |

FIG. 3 illustrates the serum HPLC profiles as analyzed by CA-PTH and T-PTH assays. Individual HPLC profiles are indicated by the light lines while the mean HPLC profiles of each group are shown as the darker, heavier line. The results are qualitatively similar in all groups. However, these results differ quantitatively from one group to the next. The quantitative planimetric evaluation of HPLC profiles is presented in Table 2 (below). The T-PTH assay identified a peak of immunoreactivity coeluting with standard hPTH(1-84) in position 45 and at least two peaks of non-(1-84)PTH in positions 36 to 41. Non-(1-84)PTH represented 17.3±6.6% of the immunoreactivity in NI, 23.8±14.4% in PHP, and 39.4±7.0% in RF. Percent non-(1-84)PTH was 2.3 times higher in RF patients than in NI. The CA-PTH assay identified a peak of immunoreactivity coeluting with hPTH(1-84) migrating in front of hPTH(1-84) at positions 42 and 43, slightly more hydrophobic than non-(1-84)PTH in positions 36 to 41. Because of the binding specificity of the CA-PTH assay that requires the presence of the first amino acid (Ser) of PTH in order to be measured, this new PTH form is N-terminally intact. The “new” PTH peak corresponded to 8.1±2.4% of CA-PTH immunoreactivity in NI, 24.7±23.3% in PHP, and 22.2±7.3% in RF. Again, pools of serum from RF patients had 2.7 times more of this new PTH molecular form than NI.

FIG. 4 represents a detailed immunological resolution analysis performed on this new molecular form of PTH with the three PTH assays using various acetonitrile HPLC gradients in the PHP patient with the highest PTH concentration. The results are expressed in absolute values to allow direct comparisons between assays. The new PTH molecular form could not be separated from hPTH(1-84) using our original HPLC gradient but was able to be resolved in the 2 newer gradients. This new form of PTH migrated differently from the non-(1-84)PTH in gradients 2 and 3, and exhibited reactivity in the CA-PTH and C-PTH assays but hardly, if at all, in the T-PTH assay (gradient 3).

Table 2 (below) also summarizes the application of the HPLC planimetric results to the original T-PTH and CA-PTH values, breaking them down into their components. hPTH(1-84) values from the T-PTH and CA-PTH assays were similar except in NI, where mean CA-PTH hPTH(1-84) was slightly lower. When non-(1-84)PTH was included along with the new amino-terminal PTH molecular form, the combined value represented 25% of the PTH measured by the two assays in NI and 50% in PHP or RF patients. In the latter population, the newer molecular form of PTH behaved by its migration similar to the non-(1-84)PTH and showed evidence of accumulation in renal failure. NI=normal individuals, PHP=primary hyperparathyroidism, RF=renal failure. Results are presented in the form of mean±SD. Statistical analysis by both ANOVA and the Student-Newman-Keuls test followed. NI vs. PHP or RF: a, p<0.001; b, p<0.01; c, p<0.05. Statistical analysis by paired Student's “t” Test. T-PTH vs. CA-PTH: d, p<01.

TABLE 2
Planimetric evaluation of HPLC profiles applied to T-PTH and CA-PTH values

| | | T-PTH assay | | | | CA-PTH assay | | | |
|---|---|---|---|---|---|---|---|---|---|
| | | Non(1-84)PTH | | PTH(1-84) | | Amino-PTH | | PTH(1-84) | |
| Groups | No | % | pmol/L | % | pmol/L | % | pmol/L | % | pmol/L |
| NI | 7 | 17.3 ± 6.6 | 0.55 ± 0.24 | 82.7 ± 6.6 | 2.58 ± 0.31 | 8.1 ± 2.4 | 0.19 ± 0.07 | 91.9 ± 2.4 | 2.10 ± 0.28^{d} |
| PHP | 5 | 23.8 ± 14.4^{b} | 7.0 ± 9.9^{c} | 76.2 ± 14.4 | 18.7 ± 19.6^{b} | 24.7 ± 23.3^{c} | 8.8 ± 15.8^{b} | 75.2 ± 23.3 | 14.4 ± 13.5^{b} |
| RF | 8 | 39.4 ± 7.0^{a} | 18.1 ± 14.7^{b} | 60.6 ± 7.0^{b} | 28.9 ± 20.7^{a} | 22.2 ± 7.3^{b} | 8.5 ± 9.3^{a} | 78.2 ± 7.8 | 25.0 ± 17.8^{a} |

II. Discussion

The present study was initiated to obtain more information on a new molecular form of circulating PTH (nfPTH) uniquely identified by the CA-PTH assay (described above) when more efficient HPLC gradients were used to separate the previously described non-(1-84)PTH from PTH(1-84).

The unique specificities of the three PTH assays, and particularly the CA-PTH assay, are important because they provide for the deduction of the structure for nfPTH. The CA-PTH assay employs a solid-phase (39-84) antibody to capture PTH, and a unique [1-4]-directed antibody to reveal hPTH(1-84) with a binding dependence on the presence of the first amino acid John M. R., et al., Journal of Clinical Endocrinology and Metabolism (1999) 84(11):4287-4290. The CA-PTH assay detects hPTH(1-84) only and a single peak of immunoreactivity coeluting with hPTH(1-84) when circulating PTH is resolved using our old HPLC gradient. See Gao P, et al., J Bone Min Res 2001;16:605-614. With more efficient gradients, the single hPTH(1-84) peak can be separated into two different entities, both having an intact amino-terminal structure that includes the first amino acid. The T-PTH assay employs the same capture antibody and a revealing antibody directed against the (15-34) region of the PTH structure. This assay detects hPTH(1-84) and hPTH(7-84) equally, and will detect the hPTH(1-84) and the non-(1-84)PTH eluted from HPLC profiles of serum derived from patients with various clinical conditions. See id. This T-PTH assay does not detect (or barely detects) the new molecular form of PTH that is recognized by the CA-PTH assay, which suggests a modification in the (15-34) region that interferes with the detection thereof via the T-PTH assay. Finally, the C-PTH assay, having its specificity in the (65-84) region and requiring an intact (65-84) PTH region for detection (Brossard J H, et al., J Clin Endocrinol Metab 1996;81:3923-3929; Brossard J H, et al., J Clin Endocrinol Metab 1993;77:413-419; Lepage R, et al., Clin Chem 1998;44:805-809; Brossard J H, et al., Clin Chem 2000;46:697-703), does not react with mid-carboxyl-terminal fragments. The C-PTH assay also detects nfPTH revealed by the CA-PTH assay, suggesting that the nfPTH possesses an intact C terminal end. These results suggest the discovery of a new form of hPTH(1-84) that is not truncated. Testing indicates that nfPTH contains a modification in the (15-34) region, as compared with normal whole human PTH. A post-translational modification of the PTH molecule was described by Rabbani et al. in 1984. See Nguyen-Yamamoto L, et al., Eur J Endocrinol 2002;147:123-131. As provided by Nguyen-Yamamoto, phosphorylation in the amino-terminal region was demonstrated using bovine and human parathyroid glands. See id. Phosphorylation of the serine residues in positions 1 and 3 would render immunoreactivity with the CA-PTH assay improbable. While not intending to be bound by theory, phosphorylation of the serine residue in position 17 provides one explanation of the inability of nfPTH to exhibit immunoreactivity in the T-PTH assay. Analyses indicate that the structure of nfPTH deviates from that of whole hPTH within a mid-terminal region, which region may comprise amino acid positions 15-34 of the hPTH sequence.

The behavior of this new PTH molecular form in the three populations studied is also of interest. Renal failure patients appear to have a higher relative amount of this new molecular form of PTH compared to that observed in NI and in patients with PHP. Two PHP patients had a proportion of this new PTH molecular form similar to that in NI, and 3 others had a much greater amount (17.8 to 63.3%). In RF patients, this new molecular PTH form was increased in the same proportion as previously reported non-(1-84)PTH (see Gao P, et al., J Bone Min Res 2001;16:605-614), suggesting an increased production and secretion by these hyper-functional parathyroid glands with subsequent accumulation in RF patients. See Monier-Fougère M C, et al., Kidney Int 2001;60:1460-1468.

Recently, it was suggested that the ratio of (1-84)/non-(1-84) PTH is a very useful index in identifying the RF patient with adynamic bone turnover from the RF patient with high/normal turnover bone condition. Such differentiation is very important in the treatment of the secondary hyperparathyroidism of RF with vitamin D analogues or by parathyroidectomy. However, the revelation of this new hPTH(1-84) with intact secondary amino acid composition should be accounted for in subjects that have nfPTH when assessing the non-(1-84)PTH level by the subtraction of the CA-PTH value from the T-PTH value. Direct evaluation of non-(1-84)PTH by the subtraction of the CA-PTH determination from the T-PTH determination might, for some patients, underestimate the total amount of non-(1-84)PTH with RF because the CA-PTH values include this new PTH molecular form which is not detected by the T-PTH assay. Moreover, the constant proportion of this new molecular form of PTH in RF patients provides an explanation regarding why Faugere, et al (see id.) were able to demonstrate with the subtraction method that the ratio of (1-84)/non-(1-84) PTH was able to discriminate the renal failure patient with adynamic low bone turnover from the renal failure patient with normal/high bone turnover disease.

As described herein, a new species of the PTH molecule with preserved intact amino acid sequences at both the N-terminal and C-termini has been identified. This new form of PTH is immunoreactive and detectable by the CA-PTH assay, but not by the T-PTH assay.

One of skill in the art would understand that although specific proprietary PTH assays have been utilized herein, the key factor for the present analysis focuses on the unique specificity of each assay within the PTH molecule. Thus, given the teachings provided herein, assays other than those provided for and utilized herein, while having similar or identical PTH molecular specificities, can be utilized.

An Exemplary PTH Assay for an Amino-Terminal Form of PTH

The present methods provide a novel 1-84 PTH (CAP) assay with a specific characteristic that it will measure PTH only if the first amino acid is present (including, e.g., serine in hPTH). In addition, another total PTH (tPTH) assay is provided herein that does not depend on the presence of the serine. In normal subjects and in patients with both primary and secondary hyperparathyroidism, it is demonstrated that the level of tPTH is higher than that of CAP. For example, we have shown that for primary hyperparathyroid patients tPTH levels are 1.3-fold higher than CAP levels (132 14 pg/ml vs 96 10 pg/ml). Thus, a substantial percentage of circulating PTH is in the form of large N-truncated fragments (likely 7-84 PTH). As parathyroid cancer is the most severe form of primary hyperparathyroidism, the relationship between tPTH and CAP was investigated.

Subjects with metastatic parathyroid cancer were selected, for example, a male of 60 yrs with a diagnosis of PT cancer for 26 years (Ca 13.4 mg/dl), and a female of 42 years with a diagnosis of PT cancer for 9 years (Ca 19.6 mg/dl, nl: 8.4-10.3). CAP and tPTH levels were measured in the male subject at 946 pgm/ml and 797 pgm/ml respectively. CAP and tPTH levels were measured in the female subject at 1619 pgm/ml and 1020 pgm/ml respectively. The fact that both patients had a higher level of CAP versus tPTH indicates that these patients have the presently described novel form of PTH (nfPTH) that contains the first amino acid serine. As both assays use the same capture antibody, the nfPTH is not an amino terminal fragment of PTH with a length of at least less than 1-44 PTH. As indicated hereinbefore, nfPTH is a full length PTH or a large N terminal fragment PTH. In either case, post translational phosphorylation/glycosylation or truncation might result in a unique three dimensional fold such that the label antibody epitope for the tPTH assay described herein is concealed. Further development of additional PTH assays, brought about the identification of nfPTH, will yield additional information on nfPTH. Elucidation of nfPTH is desirable as this will provide a further understanding of both parathyroid cancer and the overall biology of the parathyroid gland.

Role of the New Molecular Form of PTH

Further characterization and analysis of nfPTH will reveal its role in PTH mediated biological activity. For example, further studies will indicate PTH antagonist or agonist related activity of nfPTH. Moreover, further characterization and analysis of nfPTH may reveal novel activity of nfPTH as both a PTH agonist and PTH antagonist, or as playing a role in another independent biological pathway. Based on these studies, therapeutics, diagnostics, therapeutic methods, diagnostic methods and kits will be developed to utilize nfPTH based compositions and antibodies thereto. Moreover, further characterization and analysis will yield information about whether nfPTH plays an active role in PTH mediated biological activity. Design of PTH assays to either avoid or ensure detection of nfPTH are contemplated. PTH component ratios will be developed based on further studies for diagnostic and therapeutic purposes.

An Exemplary PTH Assay that Avoids Interference from Amino Regional PTH Fragments

The present disclosure additionally contemplates a new parathyroid hormone assay that avoids interference from amino regional PTH fragments. For a long time there has been difficulty associated with total intact PTH assays wherein cross-reactivity of circulating PTH fragments has differed. This observation is indicated in Table 3.

TABLE 3
Nichols Intact (IRMA) (NID) versus Scantibodies (SLI) Total
Intact PTH IRMA (SLI) in Control Samples

| PTH Parameter | NID Level Measured | SLI Level Measured |
|---|---|---|
| 1-84 PTH | 31 | 23 |
| 1-84 PTH | 394 | 329 |
| 1-84 PTH & 7-84 PTH | 128 | 158 |
| 1-84 PTH & 7-84 PTH | 447 | 522 |
| 7-84 PTH | 31 | 43 |
| 7-84 PTH | 66 | 102 |

However, in patient samples NID reads the same as SLI which happens as the SLI assay under-measures 1-84 PTH and over-measures 7-84 PTH and, generally, patient samples have a mixture of 1-84 PTH and 7-84 PTH.

It appears that there are some samples, comprising about 2% of the patient population, in which the total intact PTH (SLI) reads lower than the CAP assay (i.e., the “part” measures higher than the “total”). Exemplary PTH level data from a variety of these types of patients is provided in Table 4. In Table 4, “CAP” refers to cyclase-activating PTH levels, and “CIP” refers to cyclase inactive PTH levels (e.g., for purposes of this example, calculated via subtracting the cyclase-activating PTH level from the total PTH level).

It has previously been recognized that PTH fragments 2-34, 3-34, 4-34 and 5-34 each interfere with and result in a lower reading in SLI's total intact PTH assay (similar to NID's intact PTH assay). Thus, these observations indicate that an amino regional fragment (ARF) is present in samples exhibiting lower total PTH levels than CAP levels. An amino regional fragment, as used herein, does not include amino terminal fragment(s) (which contain the 1st amino acid). An amino regional fragment is missing the first amino acid and interferes with total PTH assays which are purified with a 1-34 PTH antibody that is not dependent on the presence of the first amino acid. The ARF has at least two utilities—first, for a total PTH assay it is important to use a PTH₂₋₃₄ up to PTH₂₈₋₃₄ (or any number of them, e.g., PTH₁₂₋₃₄ and PTH₁₃₋₃₄) to show that there is not a cross reactivity, but there must be a cross reactivity with the 7-84 PTH. For example, a antibody is made to PTH₂₀₋₃₄ which serves as a label antibody in a sandwich assay. Such antibody preferably binds PTH₇₋₈₄ such that in the absence of the amino acid in the 7th position of PTH₇₋₈₄ the antibody does not bind. Although not intended to be limited by theory, often such an antibody may have a binding specificity at least partially dependant on the 3-dimensional shape of the PTH₇₋₈₄, wherein in the absence or alteration of the amino acid in the 7th position, the antibody will not specifically bind the PTH molecule. In such a circumstance, the antibody often will not detect PTH missing the 7^{th} amino acid (or having an alteration in the 7th amino acid).

The other utility for this ARF is that it is a valuable fragment to measure, like all other PTH fragments, in order to amplify the antagonistic biological activity of 7-84 PTH. In other words, when 7-84 PTH is made—there are other PTH fragments made which may or may not have potent antagonistic biological activity, but may serve as an amplifier to make a ratio more clear.

The ordinarily skilled artisan can appreciate that the present invention can incorporate any number of the preferred features described above.

TABLE 4

| Total | CAP | CIP | | |
|---|---|---|---|---|
| PTH | PTH | Value | CAP/CIP | % Diff |
| 316.05 | 316.28 | −0.23 | −1375.13 | −0.1% |
| 802 | 806 | −4 | −201.5 | −0.5% |
| 96.05 | 96.83 | −0.78 | −124.14 | −0.8% |
| 456.97 | 461.3 | −4.33 | −106.54 | −0.9% |
| 205.75 | 207.97 | −2.22 | −93.68 | −1.1% |
| 287.28 | 290.86 | −3.58 | −81.25 | −1.2% |
| 402.14 | 407.63 | −5.49 | −74.25 | −1.4% |
| 388.65 | 394.57 | −5.92 | −66.65 | −1.5% |
| 50.67 | 51.52 | −0.85 | −60.61 | −1.7% |
| 60.93 | 62.02 | −1.09 | −56.9 | −1.8% |
| 455.59 | 464.28 | −8.69 | −53.43 | −1.9% |
| 124.39 | 127.26 | −2.87 | −44.34 | −2.3% |
| 242.42 | 248.46 | −6.04 | −41.14 | −2.5% |
| 232.52 | 240.12 | −7.6 | −31.59 | −3.3% |
| 306.1 | 316.32 | −10.22 | −30.95 | −3.3% |
| 2.42 | 2.53 | −0.11 | −23 | −4.5% |
| 190.5 | 199.74 | −9.24 | −21.62 | −4.9% |
| 78.76 | 82.64 | −3.88 | −21.3 | −4.9% |
| 170.75 | 180.9 | −10.15 | −17.82 | −5.9% |
| 16.35 | 17.36 | −1.01 | −17.19 | −6.2% |
| 6.87 | 7.3 | −0.43 | −16.98 | −6.3% |
| 113.79 | 121.1 | −7.31 | −16.57 | −6.4% |
| 412.42 | 438.99 | −26.57 | −16.52 | −6.4% |
| 262.44 | 280.25 | −17.81 | −15.74 | −6.8% |
| 137.55 | 147.09 | −9.54 | −15.42 | −6.9% |
| 116.44 | 124.9 | −8.46 | −14.76 | −7.3% |
| 246.9 | 264.93 | −18.03 | −14.69 | −7.3% |
| 213.85 | 230.71 | −16.86 | −13.68 | −7.9% |
| 98.68 | 106.62 | −7.94 | −13.43 | −8.0% |
| 127.54 | 137.99 | −10.45 | −13.2 | −8.2% |
| 91.77 | 99.39 | −7.62 | −13.04 | −8.3% |
| 240.22 | 260.66 | −20.44 | −12.75 | −8.5% |
| 63.4 | 69.04 | −5.64 | −12.24 | −8.9% |
| 173.18 | 189.05 | −15.87 | −11.91 | −9.2% |
| 220.44 | 241.66 | −21.22 | −11.39 | −9.6% |
| 330.38 | 362.29 | −31.91 | −11.35 | −9.7% |
| 137.94 | 151.51 | −13.57 | −11.17 | −9.8% |
| 160.9 | 177.48 | −16.58 | −10.7 | −10.3% |
| 730 | 806 | −76 | −10.61 | −10.4% |
| 329.68 | 364.31 | −34.63 | −10.52 | −10.5% |
| 331.81 | 366.74 | −34.93 | −10.5 | −10.5% |
| 114.97 | 127.35 | −12.38 | −10.29 | −10.8% |
| 87.8 | 97.36 | −9.56 | −10.18 | −10.9% |
| 54.1 | 60 | −5.9 | −10.17 | −10.9% |
| 533.71 | 594.25 | −60.54 | −9.82 | −11.3% |
| 318.98 | 355.52 | −36.54 | −9.73 | −11.5% |
| 18.46 | 20.6 | −2.14 | −9.63 | −11.6% |
| 110.39 | 123.55 | −13.16 | −9.39 | −11.9% |
| 82.38 | 92.82 | −10.44 | −8.89 | −12.7% |
| 227.83 | 256.93 | −29.1 | −8.83 | −12.8% |
| 396.03 | 449.94 | −53.91 | −8.35 | −13.6% |
| 245.76 | 280.88 | −35.12 | −8 | −14.3% |
| 248.82 | 285.08 | −36.26 | −7.86 | −14.6% |
| 1536.12 | 1764.16 | −228.04 | −7.74 | −14.8% |
| 164.3 | 189.06 | −24.76 | −7.64 | −15.1% |
| 209.25 | 241.57 | −32.32 | −7.47 | −15.4% |
| 426.24 | 493.87 | −67.63 | −7.3 | −15.9% |
| 112.36 | 130.67 | −18.31 | −7.14 | −16.3% |
| 469.12 | 548.6 | −79.48 | −6.9 | −16.9% |
| 216.81 | 254.58 | −37.77 | −6.74 | −17.4% |
| 32.63 | 38.38 | −5.75 | −6.67 | −17.6% |
| 1310.9 | 1546.3 | −235.4 | −6.57 | −18.0% |
| 5.01 | 5.97 | −0.96 | −6.22 | −19.2% |
| 210.68 | 251.45 | −40.77 | −6.17 | −19.4% |
| 195.4 | 233.56 | −38.16 | −6.12 | −19.5% |
| 116.31 | 139.12 | −22.81 | −6.1 | −19.6% |
| 8.24 | 9.87 | −1.63 | −6.06 | −19.8% |
| 45.74 | 54.9 | −9.16 | −5.99 | −20.0% |
| 332 | 398.68 | −66.68 | −5.98 | −20.1% |
| 85.24 | 102.84 | −17.6 | −5.84 | −20.6% |
| 76.28 | 92.74 | −16.46 | −5.63 | −21.6% |
| 1613.3 | 1980.2 | −366.9 | −5.4 | −22.7% |
| 60.62 | 74.49 | −13.87 | −5.37 | −22.9% |
| 68.26 | 84.86 | −16.6 | −5.11 | −24.3% |
| 213.09 | 265.4 | −52.31 | −5.07 | −24.5% |
| 115.92 | 144.62 | −28.7 | −5.04 | −24.8% |
| 669.57 | 835.25 | −165.68 | −5.04 | −24.7% |
| 578.69 | 724.6 | −145.91 | −4.97 | −25.2% |
| 244.1 | 307.42 | −63.32 | −4.86 | −25.9% |
| 642.22 | 822.68 | −180.46 | −4.56 | −28.1% |
| 58.13 | 75.63 | −17.5 | −4.32 | −30.1% |
| 107.34 | 142.37 | −35.03 | −4.06 | −32.6% |
| 204.22 | 277.26 | −73.04 | −3.8 | −35.8% |
| 94.17 | 128.44 | −34.27 | −3.75 | −36.4% |
| 538.04 | 733.45 | −195.41 | −3.75 | −36.3% |
| 119.2 | 163.82 | −44.62 | −3.67 | −37.4% |
| 225.09 | 312.95 | −87.86 | −3.56 | −39.0% |
| 273.34 | 383.15 | −109.81 | −3.49 | −40.2% |
| 295.1 | 419.4 | −124.3 | −3.37 | −42.1% |
| 687.43 | 987.97 | −300.54 | −3.29 | −43.7% |
| 117.87 | 172.45 | −54.58 | −3.16 | −46.3% |
| 14.76 | 21.92 | −7.16 | −3.06 | −48.5% |
| 281.48 | 420.12 | −138.64 | −3.03 | −49.3% |
| 192.63 | 292.13 | −99.5 | −2.94 | −51.7% |
| 111.7 | 169.56 | −57.86 | −2.93 | −51.8% |
| 244.97 | 372.66 | −127.69 | −2.92 | −52.1% |
| 162.35 | 250.78 | −88.43 | −2.84 | −54.5% |
| 42.6 | 66.06 | −23.46 | −2.82 | −55.1% |
| 3.99 | 6.39 | −2.4 | −2.66 | −60.2% |
| 58.8 | 99.96 | −41.16 | −2.43 | −70.0% |
| 215.45 | 366.97 | −151.52 | −2.42 | −70.3% |
| 86.95 | 150.05 | −63.1 | −2.38 | −72.6% |
| 209.42 | 368.4 | −158.98 | −2.32 | −75.9% |
| 176.21 | 314.59 | −138.38 | −2.27 | −78.5% |
| 77.48 | 143.3 | −65.82 | −2.18 | −85.0% |
| 221.39 | 413.43 | −192.04 | −2.15 | −86.7% |
| 48.04 | 100.58 | −52.54 | −1.91 | −109.4% |
| 4.5 | 9.63 | −5.13 | −1.88 | −114.0% |
| 220.84 | 508.68 | −287.84 | −1.77 | −130.3% |
| 162.98 | 430.04 | −267.06 | −1.61 | −163.9% |
| 169.94 | 488.96 | −319.02 | −1.53 | −187.7% |

### Example 6

### Removal of PTH Fragments from ESRD Patient Samples

1-84 PTH of various ESRD patient samples was measured. 1-84 PTH was then removed by incubating the samples with a bead coated with the 1-9 PTH antibody for 5 hours at room temp with rotation (1 bead with 0.55 μg Ab coated to treat 280 μl plasma). The 1-84 PTH remained in the samples was again measured. As shown in the following Table 5, about 94% of the 1-84 PTH was removed by this treatment.

TABLE 5
Removal of 1-84 PTH by antibody absorption

| | untreated | treated | |
|---|---|---|---|
| Sample 10 | (pg/ml) | (pg/ml) | % of removal |
| 46 | 259.18 | 15.25 | 94.12 |
| 47 | 185.08 | 10.12 | 94.53 |
| 48 | 239.51 | 12.15 | 94.93 |
| 49 | 206.23 | 15.83 | 92.32 |
| 50 | 228.49 | 11.04 | 95.17 |
| 51 | 256.07 | 15.2 | 94.06 |
| 52 | 1304.1 | 81.07 | 93.78 |
| 53 | 552.22 | 27.69 | 94.99 |
| 54 | 237.57 | 7.31 | 96.92 |
| 55 | 126.81 | 6.99 | 94.49 |
| 56 | 212.94 | 12.47 | 94.14 |
| 57 | 333.43 | 28.27 | 91.52 |
| 58 | 174.4 | 6.96 | 96.01 |
| 59 | 93.1 | 6.55 | 92.96 |
| 60 | 306.26 | 21.7 | 92.91 |
| 61 | 665.53 | 39.79 | 94.02 |
| 62 | 65.53 | below low | |
| | | std | |
| 63 | 524.83 | 33.44 | 93.63 |
| 64 | 655.72 | 35.53 | 94.58 |
| 65 | 160.92 | 9.01 | 94.40 |
| 66 | 508.96 | 32.28 | 93.66 |
| 67 | 135 | below low | |
| | | std | |
| 68 | 522.14 | 30.08 | 94.24 |
| 69 | 121.77 | 7.67 | 93.70 |
| 70 | 289.06 | 17.43 | 93.97 |
| 71 | 162.45 | 11.51 | 92.91 |
| 72 | 191.88 | 12.13 | 93.68 |
| 73 | 183.22 | 10.97 | 94.01 |
| average | | | 94.06 |

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A method for detecting an analyte in the presence of an interfering moiety comprising:
a) contacting a sample containing or suspected of containing an analyte and/or an interfering moiety with a blocking binding component to allow specific binding of the blocking binding component to the interfering moiety but not to the analyte, if the analyte and/or the interfering moiety is present in the sample;
b) contacting the sample with a tracer binding component to allow specific binding of the tracer binding component to the analyte but not to the interfering moiety due to the presence of the blocking binding component bound thereon; and
c) detecting the binding between the analyte and the tracer binding component to assess the presence and/or amount of the analyte in the sample,
wherein the analyte is a fragment, analog or isoform of the interfering moiety and step a) is conducted prior to or concurrently with step b).

2. The method of claim 1, wherein the interfering moiety contains an epitope that is not present, in whole or in part, on the analyte by virtue of the analyte's status as a fragment of the interfering moiety, wherein the blocking binding component is specific for this epitope.

3. The method of claim 2, wherein the interfering moiety contains another epitope that overlaps the first epitope, wherein this other epitope is present on the analyte, and wherein the tracer binding component is specific for this other epitope.

4. The method of claim 1, further comprising an assay solid phase binding component, wherein the assay solid phase binding component is contacted with the sample and allowed to specifically bind the analyte before detecting the binding between the analyte and the tracer binding component.

5. The method of claim 1, wherein the binding component aspect of the blocking binding component and/or the tracer binding component comprises an antibody, an antibody fragment, a receptor, or a member of a specific binding pair.

6. The method of claim 1, wherein the interfering moiety comprises a whole PTH and the analyte comprises a PTH fragment; wherein the interfering moiety comprises procalcitonin and the analyte comprises calcitonin; wherein the interfering moiety comprises gastric inhibitory polypeptide (GIP) and the analyte comprises glucagon-like peptide (GLP); wherein the interfering moiety comprises GIP-1 and the analyte comprises GLP-1; wherein the interfering moiety comprises GIP-2 and the analyte comprises GLP-2; wherein the interfering moiety is selected from an isoform of creatine kinase (CK) selected from the muscle (CK-MM), hybrid (CK-MB) and brain isoforms (CK-BB) and the analyte is selected from a CK isoform other than the interfering moiety; wherein the interfering moiety comprises proinsulin and the analyte comprises insulin; wherein the interfering moiety comprises osteocalcin and the analyte comprises an osteocalcin fragment; or wherein the interfering moiety comprises adrenocorticotrophic hormone (ACTH) and the analyte comprises an ACTH fragment.

7. The method of claim 1, wherein the tracer binding component further comprises a detectable label.

8. The method of claim 7, wherein the detectable label is selected from the group consisting of a substrate, a chromogen, a catalyst, a chemiluminescent compound, a particulate label, a fluorescent label, an enzymatic label, a colorimetric label, a dye label, a radioactive label, and a magnetic label.

9. The method of claim 1, wherein the analyte comprises 7-84 PTH and the interfering moiety comprises 1-84 PTH, and wherein the blocking binding component comprises an antibody having a specificity for 1-8 PTH, 1-9 PTH, 1-10 PTH, 1-11 PTH, 1-12 PTH, 1-13 PTH, 1-14 PTH, 1-15 PTH, 1-16 PTH, 1-17 PTH, 1-18 PTH, 1-19 PTH, 1-20 PTH, 1-21 PTH, 1-22 PTH, 1-23 PTH, 1-24 PTH, 1-25 PTH, 1-26 PTH, 1-27 PTH, 1-28 PTH, 1-29 PTH, 1-30 PTH, 1-31 PTH, 1-32 PTH, 1-33 PTH, 1-34 PTH, 2-8 PTH, 2-9 PTH, 2-10 PTH, 2-11 PTH, 2-12 PTH, 2-13 PTH, 2-14 PTH, 2-15 PTH, 2-16 PTH, 2-17 PTH, 2-18 PTH, 2-19 PTH, 2-20 PTH, 2-21 PTH, 2-22 PTH, 2-23 PTH, 2-24 PTH, 2-25 PTH, 2-26 PTH, 2-27 PTH, 2-28 PTH, 2-29 PTH, 2-30 PTH, 2-31 PTH, 2-32 PTH, 2-33 PTH, 2-34 PTH, 3-8 PTH, 3-9 PTH, 3-10 PTH, 3-11 PTH, 3-12 PTH, 3-13 PTH, 3-14 PTH, 3-15 PTH, 3-16 PTH, 3-17 PTH, 3-18 PTH, 3-19 PTH, 3-20 PTH, 3-21 PTH, 3-22 PTH, 3-23 PTH, 3-24 PTH, 3-25 PTH, 3-26 PTH, 3-27 PTH, 3-28 PTH, 3-29 PTH, 3-30 PTH, 3-31 PTH, 3-32 PTH, 3-33 PTH, 3-34 PTH, 4-8 PTH, 4-9 PTH, 4-10 PTH, 4-11, PTH, 4-12 PTH, 4-13 PTH, 4-14 PTH, 4-15 PTH, 4-16 PTH, 4-17 PTH, 4-18 PTH, 4-19 PTH, 4-20 PTH, 4-21 PTH, 4-22 PTH, 4-23 PTH, 4-24 PTH, 4-25 PTH, 4-26 PTH, 4-27 PTH, 4-28 PTH, 4-29 PTH, 4-30 PTH, 4-31 PTH, 4-32 PTH, 4-33 PTH, 4-34 PTH, 5-9 PTH, 5-10 PTH, 5-11, PTH, 5-12 PTH, 5-13 PTH, 5-14 PTH, 5-15 PTH, 5-16 PTH, 5-17 PTH, 5-18 PTH, 5-19 PTH, 5-20 PTH, 5-21 PTH, 5-22 PTH, 5-23 PTH, 5-24 PTH, 5-25 PTH, 5-26 PTH, 5-27 PTH, 5-28 PTH, 5-29 PTH, 5-30 PTH, 5-31 PTH, 5-32 PTH, 5-33 PTH, 5-34 PTH, 6-9 PTH, 6-10 PTH, 6-11, PTH, 6-12 PTH, 6-13 PTH, 6-14 PTH, 6-15 PTH, 6-16 PTH, 6-17 PTH, 6-18 PTH, 6-19 PTH, 6-20 PTH, 6-21 PTH, 6-22 PTH, 6-23 PTH, 6-24 PTH, 6-25 PTH, 6-26 PTH, 6-27 PTH, 6-28 PTH, 6-29 PTH, 6-30 PTH, 6-31 PTH, 6-32 PTH, 6-33 PTH, or 6-34 PTH.

10. The method of claim 4, wherein the assay solid phase binding component comprises an antibody having a specificity for the region 39-84 PTH.

11. The method of claim 4, wherein the assay solid phase comprises a microtiter plate, a glass slide, a nitrocellulose membrane, cellulose or a cellulose derivative, a latex bead, a cell, an organelle, a protein or peptide, a test tube, a plastic bead, a colloidal gold particle, a colored particle, a magnetic bead, a quantum dot, a dipstick or a screen.

12. The method of claim 1, wherein the detection of the analyte of interest comprises determining the level of the analyte in the sample.

13. The method of claim 12, wherein the analyte comprises 7-84 PTH and the interfering moiety comprises 1-84 PTH.

14. The method of claim 13, further comprising determining a total PTH level in the sample, and wherein the 7-84 PTH level is subtracted from the total PTH level to determine the level of 1-84 PTH in the sample.

15. The method of claim 14, wherein a selection of two of the 7-84 PTH level, the total PTH level and the 1-84 PTH level are compared in a ratio.

16. The method of claim 15, wherein the ratio is used to diagnose, monitor or guide treatment for a disease or disorder.

17. The method of claim 16, wherein the disease or disorder is selected from the group consisting of osteoporosis, kidney stone disease, familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, Graves disease, the extent of parathyroid gland surgical removal, oversuppression with vitamin D or a vitamin D analogue or a calcimimetic or calcium and chronic uremia.

18. A method for detecting an analyte in the presence of an interfering moiety comprising:
contacting a sample containing or suspected of containing an analyte and/or an interfering moiety with an isolation binding component to allow specific binding of the isolation binding component to the interfering moiety but not to the analyte, if the analyte and/or the interfering moiety is present in the sample, wherein the interfering moiety is removed from a solution phase in the sample by binding with the isolation binding component;
contacting the sample with a tracer binding component to allow binding of the tracer binding component to the analyte; and
detecting the binding between the analyte and the tracer binding component to assess the presence and/or amount of the analyte in the sample,
wherein the analyte is a fragment, analog or isoform of the interfering moiety and step a) is conducted prior to or concurrently with step b).

19. The method of claim 18, wherein the interfering moiety is removed from solution via the formation of an interfering moiety complex that is formed upon the contact of the sample with a complex forming binding component capable of binding with the isolation binding component.

20. The method of claim 18, further comprising contacting the sample with a nonspecific immunoglobulin composition that is derived from the same species as the isolation binding component, wherein the complex forming binding component is further capable of binding the nonspecific immunoglobulin composition to further form the interfering moiety complex.

21. The method of claim 20, wherein the isolation binding component comprises a mouse derived monoclonal antibody composition, the nonspecific immunoglobulin composition comprises mouse immunoglobulin, and the second immunoglobulin composition comprises goat anti-mouse immunoglobulin.

22. The method of claim 18, wherein the detection of the binding between the analyte and the tracer binding component comprises determining the level of analyte in the sample.

23. The method of claim 22, wherein the analyte comprises 7-84 PTH.

24. The method of claim 18, wherein the isolation binding component comprises a particle attached to a binding component.

25. The method of claim 24, wherein the particle comprises a microtiter plate, a glass slide, a nitrocellulose membrane, cellulose or a cellulose derivative, a latex bead, a cell, an organelle, a protein or peptide, a test tube, a plastic bead, a colloidal gold particle, a colored particle, a magnetic bead, a quantum dot, a dipstick or a screen.

26. The method of claim 25, wherein the bead is comprised of cellulose or cellulose derivative, a polymer, latex, glass or metal.

27. The method of claim 26, wherein the bead is cyanogen bromide activated.

28. The method of claim 18, wherein the binding component aspect of the isolation binding component, the tracer binding component, and the assay solid phase binding component each comprises an antibody, an antibody fragment, a receptor, or a member of a specific binding pair.

29. The method of claim 28, wherein the antibody is a monoclonal or polyclonal antibody.

30. The method of claim 18, wherein the binding component aspect of the isolation binding component, the tracer binding component, and the assay solid phase binding component each comprises an anti-PTH antibody.

31. The method of claim 18, wherein steps a) through d) take place in a reaction chamber.

32. The method of claim 18, further comprising contacting the sample with an assay solid phase binding component to allow specific binding of assay binding component to the analyte but not to the interfering moiety.

33. The method of claim 18, wherein the tracer binding component further comprises a detectable label.

34. The method of claim 18, wherein the analyte comprises calcitonin and the interfering moiety comprises a calcitonin precursor.

35. A method for detecting an analyte in the presence of an interfering moiety comprising:
placing a sample containing or suspected of containing an analyte and an interfering moiety in a reaction chamber;
contacting the sample with an isolation binding component, wherein the isolation binding component specifically binds the interfering moiety but not the analyte in the sample;
contacting the sample with a tracer binding component that binds the analyte and the interfering moiety in the sample;
contacting the sample with an assay solid phase binding component that binds with the analyte in the sample; and
selectively detecting the binding between the tracer binding component and: (i) the analyte, (ii) the interfering moiety, and/or (iii) the combination of the analyte and the interfering moiety,
wherein the analyte is a fragment, analog or isoform of the interfering moiety, wherein step b) is conducted prior to steps c) and/or d), and wherein the assay solid phase binding component bound to the analyte and/or the isolation binding component bound to the interfering moiety are optionally removed from the reaction chamber and into another chamber prior to selective detection of (i) the analyte or (ii) the interfering moiety.

36. The method of claim 35, wherein the analyte and the interfering moiety are present in the reaction chamber upon selective detection of the binding between the tracer binding component and the combination of the analyte and the interfering moiety.

37. The method of claim 36, wherein the analyte is removed from the reaction chamber after the sample is contacted with the isolation binding component.

38. The method of claim 37, wherein the analyte is contacted with the tracer binding component and/or the assay solid phase binding component either (i) on contact with the other reaction chamber, or (ii) subsequent to removal to the other reaction chamber.

39. The method of claim 35, wherein the isolation binding component is attached to a wall of the reaction chamber such that upon placing at least a portion of the sample in the reaction chamber, the sample contacts the isolation binding component.

40. The method of claim 35, wherein the tracer binding component is labeled with a detectable label.

41. The method of claim 40, wherein the tracer binding component comprises a first labeled tracer binding component that specifically binds the analyte and a second labeled tracer binding component that specifically binds the interfering moiety, wherein the label aspect of the first labeled tracer binding component is detectably distinguishable from the label aspect of the second labeled tracer binding component.

42. The method of claim 35, wherein detecting the analyte comprises determining the level of 7-84 PTH in the sample and detecting the interfering moiety comprises determining the level of 1-84 PTH in the sample.

43. The method of claim 42, further comprising calculating a total PTH level from the combined levels of 7-84 PTH and 1-84 PTH.

44. The method of claim 35, wherein the assay solid phase binding component bound to the analyte is removed from the reaction chamber and into another chamber prior to selective detection of the analyte or the interfering moiety, and wherein the binding between the tracer binding component and the analyte and the binding between the tracer binding component and the interfering moiety are selectively determined, wherein such determination comprises determining the level of the analyte and the level of the interfering moiety in the sample.

45. The method of claim 44, wherein the analyte comprises 7-84 PTH and the interfering moiety comprises 1-84 PTH and 1-34 PTH, wherein the method further comprises determining the level of 1-84 PTH in the sample by a direct 1-84 PTH assay.

46. The method of claim 44, wherein the level of 1-34 PTH in the sample is determined by subtracting the level of 1-84 PTH from the level of interfering moiety in the sample.

47. The method of claim 42, wherein detecting the combination of analyte and interfering moiety in the sample comprises detecting the total PTH level in the sample, and wherein the 7-84 PTH level is subtracted from the total PTH level to determine the level of 1-84 PTH in the sample.

48. The method of claim 47, wherein a selection of two of the 7-84 PTH level, the total PTH level and the 1-84 PTH level are compared in a ratio.

49. The method of claim 48, wherein the ratio is used to diagnose, monitor or guide treatment for a disease or disorder.

50. The method of claim 49, wherein the disease or disorder is selected from the group consisting of osteoporosis, kidney stone disease, familial hypocalciuria, hypercalcemia, multiple endocrine neoplasia types I and II, osteoporosis, Paget's bone disease, hyperparathyroidism, pseudohypoparathyroidism, renal failure, renal bone disease, adynamic low bone turnover renal disease, high bone turnover renal disease, osteomalacia, osteofibrosa, Graves disease, the extent of parathyroid gland surgical removal, oversuppression with vitamin D or a vitamin D analogue or a calcimimetic or calcium and chronic uremia.

51. The method of claim 35, wherein the binding component aspect of each of the isolation binding component, the tracer binding component, and the assay solid phase binding component comprises an antibody, an antibody fragment or a member of a specific binding pair.

52. The method of claim 35, wherein the binding component aspect of each of the isolation binding component, the tracer binding component, and the assay solid phase binding component comprises a monoclonal or polyclonal antibody.

53. The method of claim 35, wherein the detection of the analyte comprises determining the level of 7-84 PTH in the sample and the detection of the interfering moiety comprises determining the level of 1-84 PTH in the sample, wherein the analyte is detected in the other reaction chamber, and the interfering moiety is detected in the reaction chamber.

54. The method of claim 53, further comprising calculating a total PTH level from the combined levels of 7-84 PTH and 1-84 PTH.

55. The method of claim 54, wherein a selection of two of the 7-84 PTH level, the total PTH level and/or the 1-84 PTH level are compared in a ratio.

56. The method of claim 55, wherein the ratio is used to diagnose, monitor or guide treatment for a disease or disorder such as renal bone disease of adynamic bone disease or high bone turnover disease.

57. The method of claim 42, wherein the 7-84 PTH level in a sample is directly determined.

58. The method of claim 57, wherein the total PTH level in the subject is further determined and the total PTH level is comprised of a 1-84 PTH level, a 7-84 PTH level, and optionally PTH fragments other than 7-84 PTH.

59. A method for detecting whole PTH in a sample in the presence of PTH fragments comprising:
a) contacting a sample containing or suspected of containing whole PTH and/or a PTH fragment with a blocking binding component composition containing a binding component that specifically binds the whole PTH and the PTH fragment in the sample, such that a unique epitope present on the whole PTH, but not on the PTH fragment, is left unbound by the binding component in the blocking binding component composition;
b) contacting the sample with a tracer binding component to allow binding of the tracer binding component with the unique epitope on the whole PTH left unbound by the blocking binding component composition, wherein the tracer binding component does not bind the PTH fragment; and
c) detecting the binding between the whole PTH and the tracer binding component to assess the presence and/or amount of the whole PTH in the sample,
wherein step a) is conducted prior to step b).

60. The method of claim 59, wherein the blocking binding component composition comprises a series of monoclonal antibodies or a polyclonal antibody, and wherein the monoclonal or polyclonal antibodies are directed against all or part of a region comprising 9-34 PTH.

61. The method of claim 60, wherein the tracer binding component comprises an antibody directed against all or part of a region comprising 1-34 PTH.

62. The method of claim 59, further comprising contacting an assay solid phase binding component with the sample and allowed to specifically bind the whole PTH and/or the PTH fragment before detecting the binding between the whole and the tracer binding component.

63. The method of claim 59, wherein the tracer binding component further comprises a detectable label.

64. A composition useful for the pretreatment of a sample to determine the level of 7-84 PTH using a total PTH assay comprising a binding component specific for all or a part of a region on the PTH molecule comprising 1-9 PTH or 1-15 PTH, wherein the binding component is attached to a solid phase.

65. A composition useful for the pretreatment of a sample to determine the level of 1-84 PTH using a total PTH assay comprising a binding component specific for all or a part of a region on the PTH molecule comprising 15-34 PTH or 7-34 PTH, wherein the binding component is attached to a solid phase.

66. A method for detecting whole PTH in a sample comprising:
a) contacting a fluid sample containing or suspected of containing whole PTH and/or a PTH fragment with a tracer binding component to allow specific binding of the tracer binding component to the whole PTH;
b) contacting the sample with an isolation binding component to allow specific binding of the isolation binding component to the whole PTH;
c) contacting the sample with a nonspecific binding component, wherein the nonspecific binding component is derived from the same species as the isolation binding component;
d) contacting the sample with a complex forming binding component to allow binding of the complex forming binding component to the isolation binding component bound to the whole PTH and the nonspecific binding component to form a complex, wherein the complex precipitates out of solution; and
e) detecting the binding between the tracer binding component and the whole PTH,
wherein any or all of steps a), b) or c) are conducted prior to step d).

67. The method of claim 66, further comprising removing unbound tracer binding component, isolation binding component, nonspecific binding component and/or complex forming binding component, if any, prior to the detection of the binding between the tracer binding component and the whole PTH.

68. The method of claim 66, wherein the tracer binding component comprises an anti-1-9 PTH antibody.

69. The method of claim 68, wherein the isolation binding component comprises an anti-39-84 PTH antibody.

70. The method of claim 66, wherein the tracer binding component comprises an antibody that is derived from a different species than the isolation antibody.

71. The method of claim 66, wherein the tracer binding component further comprises a detectible label.

72. A method for detecting PTH and a fragment or analog thereof in a sample comprising:
a) contacting a sample containing or suspected of containing whole PTH and an N-terminal PTH fragment or an analog with an isolation binding component to allow specific binding of the isolation binding component to the whole PTH but not to the N-terminal PTH fragment or analog;
b) contacting the sample with an assay solid phase binding component to allow specific binding of the assay solid phase binding component to the N-terminal PTH fragment or analog;
c) contacting the sample with a tracer binding component to allow binding of the tracer binding component to the whole PTH and the N-terminal PTH fragment or analog; and
d) detecting the binding between the tracer binding component and the whole PTH, the N-terminal PTH fragment or analog and/or the combination of the whole PTH and the N-terminal PTH fragment or analog,
wherein step a) is conducted prior to steps b) and/or c).

73. The method of claim 72, wherein the N-terminal PTH fragment or analog comprises 1-34 PTH or teraparatide.

74. The method of claim 73, wherein the sample is contacted with the isolation binding component in a reaction vessel, and wherein the isolation binding component is immobilized on a surface of the reaction vessel.

75. The method of claim 72, wherein the isolation binding component comprises an anti-39-84 PTH antibody and wherein the isolation binding component binds a C-terminal PTH fragment, if present, in addition to binding whole PTH.

76. The method of claim 74, wherein the isolation binding component comprises an anti-39-84 PTH antibody, wherein the assay solid phase binding component comprises an anti-15-34 PTH antibody and the assay tracer comprises an anti-1-9 PTH antibody.

77. The method of claim 74, wherein the assay solid phase bound to the 1-34 PTH or teriparatide is removed from the reaction vessel into another vessel for detection.

78. The method of claim 77, wherein the binding between the whole PTH and the tracer binding component is detected in the reaction vessel after the assay solid phase bound to the 1-34 PTH or teriparatide is removed.

79. The method of claim 78, further comprising contacting another tracer binding component with the sample after contacting the sample with the assay solid phase binding component to allow binding of the other tracer binding component to a PTH fragment comprising 7-84 PTH, if present, in the sample, and detecting the binding between the other tracer binding component and the 7-84 PTH.

80. The method of claim 79, wherein the other tracer binding component is contacted with the sample at the same time as the primary tracer binding component.

81. The method of claim 78, wherein the detection of the binding between the whole PTH and the tracer binding component comprises determining the level of whole PTH in the sample, wherein the detection of the binding between the 1-34 PTH or teriparatide and the tracer binding component comprises determining the level of 1-34 PTH or teriparatide in the sample, and wherein a total whole PTH and 1-34 PTH or teriparatide level is calculated from the level of whole PTH and the level of 1-34 or teriparatide.

82. The method of claim 81, further comprising measuring or calculating the level of a PTH fragment comprising 7-84 PTH in the sample.

83. The method of claim 82, wherein the level of 1-34 PTH or teriparatide, whole PTH, 7-84 PTH, and/or combinations or ratios generated therefrom are utilized to diagnose, monitor or guide treatment for a disease or disorder.

84. The method of claim 83, wherein the disease or disorder comprises osteoporosis.

85. A method for detecting calcitonin in the presence of an interfering moiety comprising a calcitonin precursor in a sample comprising:
a) contacting a sample containing or suspected of containing calcitonin and/or an interfering moiety comprising a calcitonin precursor with an isolation binding component to allow specific binding of the isolation binding component to the interfering moiety but not to the calcitonin, if the calcitonin and/or the interfering moiety is present in the sample, wherein the interfering moiety is removed from a solution phase in the sample by binding with the isolation binding component;
b) contacting the sample with a tracer binding component to allow binding of the tracer binding component to the calcitonin; and
c) detecting the binding between the calcitonin and the tracer binding component to assess the presence and/or amount of the calcitonin in the sample,
wherein the step a) is conducted prior to or concurrently with step b).

86. The method of claim 85, wherein the interfering moiety is removed from solution via the formation of an interfering moiety complex that is formed upon the contact of the sample with a complex forming binding component capable of binding with the isolation binding component.

87. The method of claim 85, further comprising contacting the sample with a nonspecific immunoglobulin composition that is derived from the same species as the isolation binding component, wherein the complex forming binding component is further capable of binding the nonspecific immunoglobulin composition to form a mass of precipitate.

88. The method of claim 87, wherein the isolation binding component comprises a mouse derived monoclonal antibody composition, the nonspecific immunoglobulin composition comprises mouse immunoglobulin, and the second immunoglobulin composition comprises goat anti-mouse immunoglobulin.

89. The method of claim 85, wherein the interfering moiety comprises procalcitonin or preprocalcitonin.

90. A method for detecting a non-typical PTH (ntPTH) in a sample, comprising:
a) assaying a sample using a total PTH assay that permits detection of 1-84 PTH and 7-84 PTH, if present in the sample, to determine a total PTH level in the sample;
b) assaying the sample using a whole PTH assay that specifically detects 1-84 PTH, and also measures ntPTH, if present in the sample, to determine a combined level of 1-84 PTH and ntPTH in the sample;
c) assaying the sample to determine a 7-84 PTH level in the sample; and
d) subtracting the difference between the 7-84 level and the total PTH level from the combined level of 1-84 PTH and ntPTH to determine the ntPTH level in the sample.

91. The method of claim 90, wherein a selection of two of the 7-84 PTH level, the 1-84 PTH level, the total PTH level and/or the ntPTH level are compared in a ratio.

92. The method of claim 91, wherein the ratio is used to diagnose, monitor or guide treatment for a disease or disorder such as adynamic bone disease, high bone turnover disease or osteoporosis.

93. The composition of claim 64, further comprising a means for assaying the total PTH.

94. The composition of claim 65, further comprising a means for assaying the total PTH.

95. A method for detecting an analyte in the presence of an interfering moiety comprising:
a) contacting a sample containing or suspected of containing an analyte and/or an interfering moiety with a blocking binding component to allow specific binding of the blocking binding component to the interfering moiety but not to the analyte, if the analyte and/or the interfering moiety is present in the sample;
b) contacting the sample with an analyte analog and a binding component to allow competitive binding of the analyte and the analyte analog to the binding component wherein either the analyte analog or the binding component comprises a detectable label, and the binding component does not specifically bind to the interfering moiety due to the presence of the blocking binding component bound thereon; and
c) detecting the competitive binding of the analyte and the analyte analog to the binding component to assess the presence and/or amount of the analyte in the sample,
wherein the analyte is a fragment, analog or isoform of the interfering moiety and step a) is conducted prior to or concurrently with step b).

96. A method for detecting an analyte in the presence of an interfering moiety comprising:
a) contacting a sample containing or suspected of containing an analyte and/or an interfering moiety with an isolation binding component to allow specific binding of the isolation binding component to the interfering moiety but not to the analyte, if the analyte and/or the interfering moiety is present in the sample, wherein the interfering moiety is removed from a solution phase in the sample by binding with the isolation binding component;
b) contacting the sample with an analyte analog and a binding component to allow competitive binding of the analyte and the analyte analog to the binding component wherein either the analyte analog or the binding component comprises a detectable label; and
c) detecting the competitive binding of the analyte and the analyte analog to the binding component to assess the presence and/or amount of the analyte in the sample,
wherein the analyte is a fragment, analog or isoform of the interfering moiety and step a) is conducted prior to or concurrently with step b).
